(19) United States
(12) Patent Application Publication
(10) Pub. No.: US 2022/0041650 A1
(43) Pub. Date: Feb. 10, 2022

(54) PURIFICATION METHOD FOR RECOMBINANT PROTEINS AND NANOPARTICLES

(71) Applicant: Duke University, Durham, NC (US)

(72) Inventors: Seth R. THOMAS, Durham, NC (US); Frederick W. PORTER, Durham, NC (US); Matthew A. TYSON, Durham, NC (US); Lakshmi Sravya KOMPALLI, Durham, NC (US)

(21) Appl. No.: 17/297,931

(22) PCT Filed: Dec. 3, 2019
(86) PCT No.: PCT/US2019/064170

§ 371 Date: May 27, 2021

Related U.S. Application Data
(60) Provisional application No. 62/774,676, filed on Dec. 3, 2018.

(51) Int. Cl. C07K 1/18; B01J 41/20

(52) U.S. Cl. CPC C07K 1/18; B01J 41/20

(57) ABSTRACT

The invention is directed to methods for purifying recombinant proteins, e.g. HIV-1 envelope trimers and/or nanoparticles, wherein the methods do not use an affinity step.

The application claims the benefit and priority of U.S. Application Ser. No. 62/774,676 filed Dec. 3, 2018, the entire content of which application is herein incorporated by reference in its entirety.

This invention was made with government support under grant 5UM1 AI100663-06 and UM1-AI100645 awarded by the National Institutes of Health. The government has certain rights in the invention.

### FIELD OF USE

The invention is directed to methods for purifying recombinant proteins, e.g. HIV-1 envelope trimers and/or nanoparticles, wherein the methods do not use an affinity purification step.

### BACKGROUND OF THE INVENTION

Antibody affinity purification has been the gold standard of purifying HIV-1 envelope based trimers and nanoparticles (NPs). There is a global need to develop a non-affinity, scalable processes to purify recombinant trimeric HIV-1 envelopes, and/or nanoparticles comprising HIV-1 envelope trimers or HIV-1 based immunogens, e.g. but not limited to ferritin based nanoparticles.

### SUMMARY OF THE INVENTION

The present invention is in the field of HIV-1 recombinant envelope purification, from any suitable preparation including but not limited to any cell culture. In some embodiments, the inventive methods use orthogonal chromatography, including hydrophobic interaction chromatography, cation and anion exchange chromatography, affinity-like chromatography, and tangential flow filtration, or any combination thereof. In particular, in some embodiments the invention provides methods for large-scale production resulting in material suitable for pharmaceutical use in animals and humans.

In certain aspects the invention provides methods for purifying envelope protein, including but not limited to envelope trimers or nanoparticle complexes, wherein the methods do not comprise an affinity purification step. In certain embodiments, the recombinant molecule(s) purified by the inventive methods have particular characteristics, such as specific antigenicity and structural appearance. In other embodiments, the methods comprise use of resins which are scalable for large scale commercial purification.

In certain aspects the invention provides methods to isolate envelopes from impurities, including but not limited to product-related impurities such as product variants (e.g. but not limited to monomer, dimer, “open” trimer, misfolded trimers, free trimer, partially assembled nanoparticles, and/or aggregates), as well as process-related impurities including but not limited to host cell proteins, viral particles, and DNA. In certain embodiments, the present invention is directed to various methods which include without limitations orthogonal chromatography operations, including but not limited to hydrophobic interaction chromatography, cation and anion exchange chromatography, affinity-like chromatography, and tangential flow filtration, or any combination thereof.

The present invention relates to methods for isolating HIV-1 envelope from recombinant cell culture utilizing orthogonal chromatography, including hydrophobic interaction chromatography, cation and anion exchange chromatography, affinity-like (mixed-mode) chromatography, lectin affinity chromatography, and tangential flow filtration, or any combination thereof. These techniques are useful individually to clear impurities, including but not limited to product-related impurities such as product variants (e.g. but not limited to monomer, dimer, “open” trimer, misfolded trimers, free trimer, partially assembled nanoparticles, and/or aggregates), as well as other process-related impurities including but not limited to host cell proteins, viral particles, and DNA. When combined in a particular sequence, specifically including hydrophobic interaction chromatography, product variants including undesired, improperly folded HIV-1 envelope are additionally removed.

In certain aspects, the invention provides recombinant HIV-1 envelope comprised in a trimer or a nanoparticle, wherein the envelope is purified by the methods of the invention.

In certain aspects, the invention provides methods to purify recombinant HIV-1 envelope trimer comprising the steps of Method 1, Method 2, or Method 3 in FIG. 1A or a combination of steps from Method 1, 2 and/or 3. In certain embodiments, the method comprises additional steps, including but not limited to the steps listed in FIG. 1A or 1B. In non-limiting embodiments, the method comprises the steps listed in FIG. 2. In non-limiting embodiments, the method comprises the steps listed in FIG. 7. In non-limiting embodiments, the method comprises the steps listed in FIG. 12. In certain aspects, the invention provides a method to purify recombinant HIV-1 envelope trimer comprising the steps of the method in FIG. 21A or 21B.

In certain aspects, the invention provides methods to purify recombinant HIV-1 envelope trimer consisting essentially of the steps of Method 1, Method 2, or Method 3 in FIG. 1A or a combination of steps from Method 1, 2 and/or 3. In certain embodiments, the method comprises additional steps, including but not limited to the steps listed in FIG. 1A or 1B. In non-limiting embodiments, the method comprises the steps listed in FIG. 2. In non-limiting embodiments, the method comprises the steps listed in FIG. 7. In non-limiting embodiments, the method comprises the steps listed in FIG. 12. In certain aspects, the invention provides a method to purify recombinant HIV-1 envelope trimer comprising the steps of the method in FIG. 21A or 21B.

In certain aspects, the invention provides a method to purify recombinant HIV-1 envelope trimer multimerized in a nanoparticle, the method comprising the steps in FIG. 22A or 22B, wherein in certain embodiments the TFF step is optional. In certain aspects, the invention provides a method to purify recombinant HIV-1 envelope trimer multimerized in a nanoparticle, the method consisting essentially the steps in FIG. 22A or 22B, wherein in certain embodiments the TFF step is optional.

In certain aspects the invention provides methods of purifying recombinant nanoparticles comprising HIV-1 envelope, the method comprising (a) a multi-mode chromatography step, for example but not limited to a chromatographic step using Capto Core 700 resin (or a functional equivalent thereof) and (b) an anion exchange chromatographic step, for example but not limited to a chromatographic step using Toyopearl NH2 750F resin (or a functional equivalent thereof), wherein the method does not comprise an affinity (lectin or antibody) based chromatographic step. In non-limiting embodiments, the method comprises: step (a) contacting a Capto Core 700 resin (or a functional equivalent thereof) with a fraction (1) containing recombinant nanoparticles, step (b) recovering the flow through fraction (2), step (c) contacting Toyopearl NH2 750F resin (or a functional equivalent thereof) with the flow through fraction (2) of step (b), and step (d) eluting a fraction (3) from the resin of step (c), wherein the eluted fraction (3) is enriched for the recombinant nanoparticle compared to fraction (1).

In certain embodiments, fraction (3) is enriched for the recombinant nanoparticle compared to fraction (1) or fraction (2). In certain embodiments, the eluted fraction (3) comprises substantially less product-related impurities compared to fraction (1) or fraction (2).

The methods of the invention combine chromatographic resins and conditions that leverage charge and hydrophobicity differences to remove impurities including product-related impurities and host cell proteins (HCP), without using traditional affinity (e.g. antibody and lectin) purification steps, used for the purification of envelope proteins. A skilled artisan appreciated that both product-related impurities, as well as process-related impurities such as HCP and/or host cell viruses are removed by the various chromatographic steps.

In certain embodiments, the methods comprise additional steps, whereby the method is GMP compliant and leads to the purification of a recombinant envelope and/or nanoparticle suitable for use as a drug substance.

In certain embodiments, the methods of the invention do not comprise affinity (e.g. lectin or antibody) based chromatographic step.

In certain aspects the invention provides methods which comprise or consist essentially of three chromatographic steps for purification of recombinant viral envelope protein: an AEX chromatography step, a mixed mode chromatography step, and/or a HIC chromatography step.

In certain aspects, the invention provides methods of purifying a recombinant viral envelope protein, the method comprising or consisting essentially of:
a. step (a) contacting an anion exchange (AEX) chromatography resin, e.g. but not limited to a Toyopearl NH2 750F resin (or a functional equivalent thereof) with a fraction (1) comprising recombinant viral envelope protein,
b. step (b) eluting a fraction (2) from the resin of step (a), wherein fraction (2) has fewer product-related impurities compared to fraction (1)
c. step (c) contacting a mixed mode chromatography resin, e.g. but not limited to Ceramic Hydroxyapatite (CHT) resin, such as CHT type 1 40 μm resin (or a functional equivalent thereof) with the fraction (2) from step (b), and
d. step (d) eluting a fraction (3) from the resin of step (c),
   i. wherein fraction (3) has fewer product-related impurities compared to fraction (1) and/or fraction (2).

In certain embodiments, the methods further comprises:
e. step (e) contacting a HIC resin, e.g. but not limited to Capto Phenyl resin (or a functional equivalent thereof) under suitable conditions, e.g. for flow through of the purified protein in fraction (3), with fraction (3) from step (d), and
f. step (f) collecting unbound flow through and/or a first resin wash as fraction (4) under suitable conditions,
   i. wherein fraction (4) has fewer product-related impurities compared to fraction (1), fraction (2) and/or fraction (3), and
   ii. wherein the method does not comprise an affinity (lectin or antibody) based chromatographic step.

In certain aspects the invention provides methods of purifying a recombinant viral envelope protein, the method comprising or consisting essentially of:
a. step (a) contacting an anion exchange (AEX) chromatography resin, e.g. but not limited to a Toyopearl NH2 750F resin (or a functional equivalent thereof) with a fraction (1) comprising a recombinant viral envelope protein,
b. step (b) eluting a fraction (2) from the resin of step (a), wherein fraction (2) has fewer product-related impurities compared to fraction (1)
c. step (c) contacting a HIC resin, e.g. but not limited to Capto Phenyl resin (or a functional equivalent thereof) with fraction (2) from step (b), and
d. step (d) collecting flow through from step (c) as fraction (3),
   i. wherein fraction (3) has fewer product-related impurities compared to fraction (1) and/or fraction (2).

In certain aspects the invention provides methods of purifying a recombinant viral envelope protein, the method comprising or consisting essentially of:
a. step (a) contacting a Mixed-mode chromatography resin, e.g. but not limited to Capto DeVirS resin (or a functional equivalent thereof) with a fraction (1) comprising a recombinant viral envelope protein,
b. step (b) eluting fraction (2) from the resin of step (a), wherein fraction (2) has fewer product-related impurities compared to fraction (1),
c. step (c) contacting a HIC resin, e.g. but not limited to Phenyl sepharose resin (or a functional equivalent thereof) with fraction (2) from step (b), and
d. step (d) eluting a fraction (3) from the resin of step (c),
   i. wherein fraction (3) has fewer product-related impurities compared to fraction (1) and/or fraction (2).

In certain aspects the methods further comprise a viral reduction step.

In certain aspects the invention provides methods of purifying a recombinant nanoparticle comprising a recombinant viral envelope protein, the method comprising or consisting essentially of:
a. step (a) contacting a multi-mode resin, e.g. but not limited to Capto Core 700 resin (or a functional equivalent thereof) with a fraction (1) comprising recombinant nanoparticles,
b. step (b) collecting a flow through from step (a) as fraction (2),
c. step (c) contacting an anion exchange (AEX) chromatography resin, e.g. but not limited to a Toyopearl NH2 750F resin (or a functional equivalent thereof) with the flow through fraction (2), and
d. step (d) eluting a fraction (3) from the resin of step (c),
   i. wherein fraction (3) has fewer product-related impurities compared to fraction (1) and/or fraction (2).

In certain aspects, the methods further comprise:
e. step (e) contacting a HIC resin, e.g. but not limited to Capto Phenyl resin (or a functional equivalent thereof), under conditions suitable for flow through operation or binding to the HIC resin, with fraction (3) from step (d), and
f. step (f) collecting unbound flow through as fraction (4) under suitable conditions (if flow through operation conditions are used in step (e)) or eluting a fraction (4) under suitable conditions (if binding conditions are used in step (e)),
   i. wherein fraction (4) has fewer product-related impurities compared to fraction (1), fraction (2) and/or fraction (3), and
   ii. wherein the method does not comprise an affinity (lectin or antibody) based chromatographic step.

In certain aspects, the methods further comprise:
e. step (e) contacting a mixed-mode resin, e.g. but not limited to CHT (or a functional equivalent thereof) under suitable conditions with fraction (3) from step (d), and
f. step (f) eluting a fraction (4) under suitable conditions,
   i. wherein fraction (4) has fewer product-related impurities compared to fraction (1), fraction (2) and/or fraction (3), and
   ii. wherein the method does not comprise an affinity (lectin or antibody) based chromatographic step.

In certain aspects, the recombinant viral envelope protein is a membrane glycoprotein from an enveloped virus, for example, but not limited to, HIV-1 envelope glycoprotein. In certain embodiments, the recombinant viral envelope protein is an HIV-1 envelope protein, wherein the HIV-1 envelope protein comprise a gp140 sequence designed to form a stable trimer. In certain aspects, the recombinant viral envelope protein is from any other virus which comprises viral glycoprotein(s).

In certain aspects, the recombinant viral envelope protein is CH505 T/F trimer. In certain embodiments, the recombinant viral envelope protein is CH505 T/F SOSIP 4.1 (FIG. 32).

In certain aspects, the AEX resin is contacted with fraction (1) in 250 mM salt buffer.

In certain aspects, fraction (2) is eluted from the AEX resin in 600 mM salt buffer.

In certain aspects, the mixed-mode resin is contacted with fraction (2) in 600 mM salt buffer.

In certain aspects, fraction (3) is eluted from the mixed-mode resin is in 30 mM phosphate buffer.

In certain aspects, the HIC resin is contacted with fraction (3) in 600 mM ammonium sulfate.

In certain aspects, the flow through fraction (4) is collected in 600 mM ammonium sulfate.

In certain aspects, all steps are conducted at pH 7.0-7.4.

In certain aspects, fraction (3) or fraction (4) comprises a well-folded trimer.

In certain aspects, fraction (3) or fraction (4) comprises a nanoparticle comprising well-folded trimers.

In certain aspects, the methods further comprise at least one viral reduction step.

In certain aspects, fraction (1) comprises a harvest pool from a bioreactor culture of 20 L to 20,000 L.

In certain aspects, the fraction (1) comprises a harvest pool that has been subjected to a Tangential Flow Filtration (TFF) step.

In certain aspects the invention provides-methods of purifying recombinant trimer comprising HIV-1 envelope, the method comprising or consisting essentially of:
a. Step (a) subjecting a fraction (1) to an anion exchange (AEX) chromatographic step under suitable conditions, e.g. but not limited to a chromatographic step using Toyopearl NH2 750F resin (or a functional equivalent thereof),
b. Step (b) recovering fraction 2 from the resin of step (a), wherein fraction (2) has fewer product-related impurities compared to fraction (1),
c. Step (c) subjecting fraction (2) from step (b) to HIC, e.g. but not limited to Capto Phenyl resin (or a functional equivalent thereof) under suitable conditions, and
d. Step (d) recovering fraction (3) from the resin of step (c),
   i. wherein fraction (3) has fewer product-related impurities compared to fraction (1) and fraction (2), and
   ii. wherein the method does not comprise an affinity (lectin or antibody) based chromatographic step.

A non-limiting embodiment is shown in Example 1, FIG. 7, and accompanying description and tables.

In certain aspects the invention provides-methods of purifying a recombinant trimer comprising HIV-1 envelope, the method comprising:
a. step (a) contacting an anion exchange (AEX) chromatography resin, e.g. but not limited to a Toyopearl NH2 750F resin (or a functional equivalent thereof) under suitable conditions with a fraction (1) comprising recombinant trimer,
b. step (b) eluting fraction (2) from the resin of step (a) under suitable conditions, wherein fraction (2) has fewer product-related impurities compared to fraction (1)
c. step (c) contacting a Ceramic Hydroxyapatite (CHT) resin, e.g. but not limited to CHT type 1 40 μm resin (or a functional equivalent thereof) under suitable conditions with the fraction (2) from step (b), and
d. step (d) eluting fraction (3) from the resin of step (c) under suitable conditions,
   i. wherein fraction (3) has fewer product-related impurities compared to fraction (1) and/or fraction (2), and
   ii. wherein the method does not comprise affinity (lectin or antibody) based chromatographic step.
      In certain embodiments the eluted fraction (3) is enriched for the recombinant trimer compared to fraction (1) and/or fraction (2). In certain embodiments, the eluted fraction (3) is substantially free of product-related impurities. In certain embodiments, the eluted fraction (3) had fewer product-related impurities compared to fraction (1) and/or fraction (2).

A non-limiting embodiment is shown in Example 1, FIG. 2, and accompanying description and tables.

In certain aspects the invention provides-methods of purifying a recombinant trimer comprising HIV-1 envelope, the method comprising:
a. step (a) contacting an anion exchange (AEX) chromatography resin, e.g. but not limited to a Toyopearl NH2 750F resin (or a functional equivalent thereof) under suitable conditions with a fraction (1) comprising recombinant trimer,
b. step (b) eluting fraction (2) from the resin of step (a) under suitable conditions, wherein fraction (2) has fewer product-related impurities compared to fraction (1)
c. step (c) contacting a HIC resin, e.g. but not limited to Capto Phenyl resin (or a functional equivalent thereof) under suitable conditions with fraction (2) from step (b), and
d. step (d) collecting unbound flow through fraction (3) from step (c) under suitable conditions,
   i. wherein fraction (3) has fewer product-related impurities compared to fraction (1) and fraction (2), and
   ii. wherein the method does not comprise an affinity (lectin or antibody) based chromatographic step.
      In certain embodiments the eluted fraction (3) is enriched for the recombinant trimer compared to fraction (1) and/or fraction (2). In certain embodiments, the eluted fraction (3) is substantially free of product-related impurities. In certain embodiments, the eluted fraction (3) had fewer product-related impurities compared to fraction (1) and/or fraction (2).

A non-limiting embodiment is shown in Example 1, FIG. 7, and accompanying description and tables.

In certain aspects the invention provides-methods of purifying a recombinant trimer comprising HIV-1 envelope, the method comprising:
a. step (a) contacting a mixed-mode chromatography resin, e.g. but not limited to Capto DeVirS resin (or a functional equivalent thereof) under suitable conditions with a fraction (1) comprising recombinant trimer,
b. step (b) eluting fraction (2) from the resin of step (a) under suitable conditions, wherein fraction (2) has fewer product-related impurities compared to fraction (1)
c. step (c) contacting a HIC resin, e.g. but not limited to Phenyl sepharose resin (or a functional equivalent thereof) under suitable conditions with fraction (2) from step (b), and
d. step (d) eluting fraction (3) from the resin of step (c) under suitable conditions,
   i. wherein fraction (3) has fewer product-related impurities compared to fraction (1) and/or fraction (2), and
   ii. wherein the method does not comprise an affinity (lectin or antibody) based chromatographic step.
      In certain embodiments the eluted fraction (3) is enriched for the recombinant trimer compared to fraction (1) and/or fraction (2). In certain embodiments, the eluted fraction (3) is substantially free of product-related impurities. In certain embodiments, the eluted fraction (3) had fewer product-related impurities compared to fraction (1) and/or fraction (2).

A non-limiting embodiment is shown in Example 1, FIG. 12, and accompanying description and tables.

In certain aspects the invention provides-methods of purifying a recombinant trimer comprising HIV-1 envelope, the method comprising:
a. step (a) contacting an anion exchange (AEX) chromatography resin, e.g. but not limited to a Toyopearl NH2 750F resin (or a functional equivalent thereof) under suitable conditions with a fraction (1) comprising recombinant trimer,
b. step (b) eluting fraction (2) from the resin of step (a) under suitable conditions, wherein fraction (2) has fewer product-related impurities compared to fraction (1)
c. step (c) contacting a Ceramic Hydroxyapatite (CHT) resin, e.g. but not limited to CHT type 1 40 μm resin (or a functional equivalent thereof) under suitable conditions with the fraction (2) from step (b),
d. step (d) eluting fraction (3) from the resin of step (c) under suitable conditions,
e. step (e) contacting a HIC resin, e.g. but not limited to Capto Phenyl resin (or a functional equivalent thereof) under suitable conditions with fraction with fraction (3) from step (c), and
f. step (f) collecting unbound flow through fraction (4) under suitable conditions,
   i. wherein fraction (4) has fewer product-related impurities compared to fraction (1), fraction (2) and/or fraction (3), and
   ii. wherein the method does not comprise an affinity (lectin or antibody) based chromatographic step.

A non-limiting embodiment is shown in Example 1, FIGS. 21A-B, and accompanying description and tables.

In non-limiting embodiments, the methods of the invention comprise a viral reduction step, such as, but not limited to, viral inactivation and/or viral filtration step. In non-limiting embodiments, a viral filtration step is carried out after the CHT step and before the HIC step. In these embodiments, the HIC resin is contacted with the fraction from the viral reduction step.

In non-limiting embodiments, the invention provides a method of purifying a recombinant trimer comprising HIV-1 envelope, the method comprising:
a. step (a) contacting an anion exchange (AEX) chromatography resin, e.g. but not limited to a Toyopearl NH2 750F resin (or a functional equivalent thereof) under suitable conditions with a fraction (1) comprising recombinant trimer,
b. step (b) eluting fraction (2) from the resin of step (a) under suitable conditions, wherein fraction (2) has fewer product-related impurities compared to fraction (1)
c. step (c) contacting a Ceramic Hydroxyapatite (CHT) resin, e.g. but not limited to CHT type 1 40 μm resin (or a functional equivalent thereof) under suitable conditions with the fraction (2) from step (b),
d. step (d) eluting fraction (3) from the resin of step (c) under suitable conditions,
e. step (e) subjecting fraction (3) from step (c) to viral reduction, e.g. but not limited to nanofiltration, and collecting fraction (3.1), wherein the viral load of fraction (3.1) is reduced compared to the viral load of fraction (3);
f. step (e) contacting a HIC resin, e.g. but not limited to Capto Phenyl resin (or a functional equivalent thereof) under suitable conditions with fraction (3.1) from step (e), and
g. step (g) collecting unbound flow through fraction (4) under suitable conditions,
   i. wherein fraction (4) has fewer product-related impurities compared to fraction (1), fraction (2) and/or fraction (3), and
   ii. wherein the method does not comprise an affinity (lectin or antibody) based chromatographic step.

In non-limiting embodiments, HIC chromatography is conducted under suitable conditions, wherein suitable conditions could include flow-through operation of the HIC resin or bind and elute operation.

In certain aspects the invention provides-methods of purifying a recombinant nanoparticle comprising HIV-1 envelope, the method comprising:
a. step (a) contacting a multi-mode resin, e.g. but not limited to Capto Core 700 resin (or functional equivalent thereof) under suitable conditions with a fraction (1) comprising recombinant nanoparticle,
b. step (b) recovering a flow through fraction (2) from step (a),
c. step (c) contacting an anion exchange (AEX) chromatography resin, e.g. but not limited to a Toyopearl NH2 750F resin (or a functional equivalent thereof) under suitable conditions with the flow through fraction (2), and
d. step (d) eluting fraction (3) from the resin of step (c),
   i. wherein fraction (3) has fewer product-related impurities compared to fraction (1) and/or fraction (2), and
   ii. wherein the method does not comprise an affinity (lectin or antibody) based chromatographic step.

In certain embodiments, the method further comprises one or more additional chromatography steps. In certain embodiments, the method comprises: step (e) contacting a CHT resin, e.g. but not limited to CHT type 1 40 μm resin (or a functional equivalent thereof) under suitable conditions with the fraction (3) from step (c); step (f) eluting fraction (4) from the resin of step (e) under suitable conditions,
i. wherein fraction (4) has fewer product-related impurities compared to fraction (1) and fraction (2) and/or fraction (3), and
ii. wherein the method does not comprise an affinity (lectin or antibody) based chromatographic step.

In certain embodiments, the method further comprises one or more additional chromatography steps. In certain embodiments, the method comprises: step (e) contacting a HIC resin, e.g. but not limited to Capto Phenyl resin (or a functional equivalent thereof) under suitable conditions with the fraction (3) from step (c), wherein the conditions for contacting the HIC resin could include binding conditions or flow through conditions; step (f) eluting fraction (4) from the resin of step (e) under suitable conditions (if binding conditions are used), or alternative step (f) collecting unbound flow through fraction (4) under suitable conditions (if flow through conditions are used),
i. wherein fraction (4) has fewer product-related impurities compared to fraction (1) and fraction (2) and/or fraction (3), and
ii. wherein the method does not comprise an affinity (lectin or antibody) based chromatographic step.

A non-limiting embodiment is shown in Example 1, FIGS. 22A-B, and accompanying description and tables.

In non-limiting embodiments, the nanoparticle purification methods comprise one or more additional chromatography operations and/or steps for generating material suitable for clinical use. The one or more additional chromatography operations and/or steps will be used to demonstrate viral clearance, to lower process-related impurities such as host cell contaminants, and/or to lower product-related impurities. Without being bound by theory, and based on observation from the non-affinity trimer process, the glycoprotein head of the nanoparticle will provide binding capability to mixed-mode resins, such as, but not limited to, Capto Core resins, CHT, and binding or optimized flow through operation of hydrophobic interaction chromatography (HIC) resins. In addition, a dedicated viral clearance operation may be required. A non-limiting embodiment of a GMP nanoparticle purification process is shown in FIG. 22B.

In non-limiting embodiments, suitable conditions allow for interaction between the resin and the fraction loaded on to the resin, wherein the product, such as a trimer or nanoparticle, is retained on the resin. In non-limiting embodiments, suitable conditions allow for interaction between the resin and the fraction loaded on to the resin, wherein the product, such as a trimer or nanoparticle, flows through and is recovered in a flow-through fraction.

In certain embodiments, the fraction (1) is a clarified harvest from cell culture, e.g. CHO cells expressing envelope trimer or nanoparticle. In certain embodiments, the clarified harvest could be subjected to concentration, e.g. but not limited to TFF. In certain embodiments, the clarified harvest could be subject to viral inactivation steps.

In certain embodiments, the methods comprise a TFF step prior to the AEX step. In certain embodiments, the methods comprise a viral inactivation step prior to the AEX step.

In certain embodiments, fraction (3 or 4) comprises a well folded trimer. In certain embodiments, fraction (3 or 4) comprises a nanoparticle comprising well folded trimers. In certain embodiments, fraction (3 or 4) is enriched for the recombinant nanoparticle compared to the fraction (1). In certain embodiments, fraction (3 or 4) is enriched for the recombinant nanoparticle compared to fraction (2). In certain embodiments, fraction (3 or 4) comprises substantially less product-related impurities, such as but not limited to monomers, dimer, “open” trimer, misfolded trimer, free trimer, partially assembled nanoparticles and/or aggregates, compared to the fraction (1) or fraction (2).

In certain embodiments, fraction (3 or 4) comprises purified product, trimer or nanoparticle, which is qualitatively comparable to a product purified through affinity chromatography, e.g. but not limited to antibody affinity chromatography.

In non-limiting embodiments, product quality is characterized by antigenicity analyses. In non-limiting embodiments, a well folded product is characterized by relative binding to broad neutralizing antibodies (bnAbs). In non-limiting embodiments, a well folded product is characterized by lack of relative binding to certain non-broad neutralizing antibodies. In non-limiting embodiments, a well folded product is characterized by image analysis, e.g. by NS-EM.

In certain embodiments, the methods are adapted for large scale recombinant protein purification.

In certain embodiments, the methods are adapted for GMP compliant protein purification.

All steps are performed under conditions suitable for interaction between a resin and trimer or nanoparticle. In certain embodiments, the method of purifying recombinant trimer or nanoparticle consists essentially of the steps described in any of the preceding paragraphs.

In certain embodiments, the methods comprise any suitable wash steps, e.g, but not limited between step (a) and step (b), step (b) and step (c), step (c) and step (d).

In certain embodiments, the fraction (1) is a harvest fraction which has been treated by TFF.

In certain embodiments, the method of any of the preceding paragraphs is suitable for large scale production. Non-limiting examples of large scale production include supernatant from 1,000 L up to 20,000 L cell culture.

In certain embodiments, the nanoparticle of the method of any of the preceding paragraphs has a size range of less than 150, 140, 130, 120, 110, 100, 90, 80, 70, 60, 50, or 40 nanometers. In certain embodiments, the nanoparticle of the method of any of the preceding paragraphs has a size range of 25-35 nanometers, 30-40 nanometers, 30-50 nanometers, 30-60 nanometers, 30-70 nanometers, 30-80 nanometers, 30-90 nanometers, 30-100 nanometers, 30-110 nanometers, 30-120 nanometers, 30-130 nanometers, 30-140 nanometers, or 30-150 nanometers.

In certain embodiments, the method of any of the preceding paragraphs further comprises a Tangential Flow Filtration (TFF) step carried out prior to the multi-mode step. In certain embodiments, the TFF steps is a UFDF filtration step.

In certain aspects, the invention provides a method to purify a recombinant HIV-1 envelope trimer comprising or consisting essentially of an AEX step, a CHT step, and a HIC step, as shown in FIG. 21A or 21B and Example 1. In certain embodiments, the method comprises all the steps in FIG. 21A or 21B. In non-limiting embodiments, the purification methods of the invention comprise a capture chromatography step (AEX), an intermediate chromatography step (CHT), and a polishing chromatography step (HIC). In non-limiting embodiments, the purification methods of the invention consist essentially of a capture chromatography step (AEX), an intermediate chromatography step (CHT), and a polishing chromatography step (HIC). In non-limiting embodiments, the purification methods of the invention comprises the following steps: an initial Tangential Flow Filtration (TFF) step, which step is optional in certain embodiments; a viral inactivation step, which step is optional in certain embodiments; a capture chromatography step (AEX, e.g. Tosoh NH2-750); an intermediate chromatography step (CHT); a nanofiltration step, which step is optional in certain embodiments); a polishing chromatography step (HIC, e.g. Capto Phenyl); and a UFDF step, which step is optional in certain embodiments.

In certain aspects, the invention provides a method to purify a recombinant HIV-1 envelope trimer in a nanoparticle, the method comprising or consisting essentially of a mixed-mode chromatography step, an AEX step, and a polishing chromatography step, e.g. but not limited to a CHT step and/or a HIC step as shown in FIGS. 22A-B and Example 1.

The steps of contacting resins in the various chromatographic steps of the inventive methods are conducted under suitable conditions for binding of the protein of interest or flow through of the protein of interest, such as but not limited to buffer pH, conductivity, salt, and so forth. The steps of eluting product from the resins or collecting flow through or wash fractions in the various chromatographic steps of the inventive methods are conducted under suitable conditions, such as but not limited to buffer pH, conductivity, salt, and so forth. Suitable conditions for the various steps are readily determined by routine optimization.

In certain embodiments, the methods comprise additional steps, such as virus clearance, etc. as required for GMP production of a biologic product.

Non-limiting embodiments of buffers used in the chromatography steps of the invention are shown in Example 1.

Cell cultures used for the production of protein, including viral envelopes, for use in pharmaceutical applications include without limitation mammalian cells such as CHO cells, NSO cells, Sp2/0 cells, COS cells, HEK cells, BHK cells, PER.C6® cells, and the like.

### BRIEF DESCRIPTION OF THE DRAWINGS

The patent or application file contains at least one drawing executed in color. To conform to the requirements for PCT patent applications, many of the figures presented herein are black and white representations of images originally created in color.

FIG. 1A shows three variations of purification methods for gp140 trimers. FIG. 1B shows one embodiment of a purification method for gp140 trimers. In non-limiting embodiments, the methods comprise any additional steps, including but not limited to TFF of the clarified harvest.

FIG. 2 shows purification Method 1 utilizing anion exchange (AEX) Toyopearl NH2 750F and ceramic hydroxyapatite (CHT) CHT Type 1 40 μm.

FIG. 3 shows non-reduced SDS-PAGE results showing high purity of gp140 in the ceramic hydroxyapatite chromatography (CHT) product pool. Lane 1 is the molecular weight standards, Lane 2 shows the NH2 product pool, lane 3 is the unbound CHT FT/wash fraction, and lane 4 is the CHT product pool.

FIG. 4 shows size exclusion chromatography (SEC) of the ceramic hydroxyapatite chromatography (CHT) pool. Peak shaded in blue indicates a uniform, trimeric gp140 population.

FIG. 5 shows (Left) a Negative Stain Electron Microscopy (NS-EM) raw image showing purified trimer molecules. (Right) 15170 images were classified into 32 classes and results are shown with all classes indicating well-formed trimer.

FIG. 6 shows antigenicity characterization of the ceramic hydroxyapatite chromatography (CHT) product pool by biolayer interferometry (BLI). The graph shows expected binding profile of trimer binding to bnAbs PGT151, PGT145, 2G12, CH106 and VRC01 but not to antibodies that define non-neutralizing binding epitopes 17B (CCR5 binding site) or 19B (V3 loop).

FIG. 7 shows purification Method 2 utilizing anion exchange (AEX) Toyopearl NH2 750F and hydrophobic interaction chromatography (HIC) Capto Phenyl

FIG. 8 shows non-reduced SDS-PAGE results showing high purity of gp140 in the Phenyl product pool (lane 3). Lane 1 is the molecular weight standards. Lane 2 shows NH2 Product Pool, lane 3 is the HIC Capto Phenyl Product Pool, and lane 4 shows impurities removed in the H2O Strip of the Capto Phenyl.

FIG. 9 shows SEC of the HIC Capto Phenyl Product Pool. The peak shaded in blue indicates a uniform, trimeric gp140 population.

FIG. 10 shows (Left) a NS-EM raw image showing purified trimer molecules. (Right) 10344 particles were classified into 32 classes and results are shown with only 1 class scored as non-trimer at 1.6% of the total.

FIG. 11 shows antigenicity characterization of the HIC Capto Phenyl Product Pool by Biolayer Interferometry (BLI). The graph shows expected binding profile of trimer binding to bnAbs PGT151, PGT145, 2G12, CH106 and vRC01 but not to non-neutralzing Abs 17B (CCR5 binding site) or 19B (V3 loop).

FIG. 12 shows purification Method 3 utilizing mixed-mode Capto DeVirS and hydrophobic interaction chromatography (HIC) Phenyl Sepharose FF.

FIG. 13 shows non-reduced SDS-PAGE results showing high purity of gp140 in the HIC Phenyl Sepharose Product Pool (lane 4). Lane 1 shows the starting clarified harvest, Lane 2 shows the tangential flow filtration (TFF) product pool, Lane 3 shows the Capto DeVirS Product Pool, and Lane 5 shows the HIC Phenyl Sepharose Product Pool. In lane 6, the molecular weight standards are shown.

FIG. 14 shows SEC of the HIC Phenyl Sepharose Product Pool. The result shows a uniform trimeric gp140 population in the HIC Phenyl Sepharose Product Pool.

FIG. 15 shows (Left) a NS-EM raw image showing purified trimer molecules. (Right) 16654 particles were classified into 32 classes and results are shown with only 1 class scored as non-trimer at 2.9% of the total.

FIG. 16 shows antigenicity characterization of the HIC Phenyl Sepharose Product Pool by Biolayer Interferometry (BLI). The graph shows expected binding profile of trimer binding to bnAbs PGT151, PGT145, PGT128, PGT125, 2G12, VRC01, CH103UCA, and CH106 but not to non-neutralizing Abs 17B (CCR5 binding site), 19B (V3 loop), F39F, CH58, A32, F105.

FIG. 17 shows non-reduced SDS-PAGE results showing the high gp140 purity in the HIC Capto Phenyl Product Pool (Lane 5). Molecular weight standards are shown in Lane 1 and Lane 6. Lane 2 shows the starting clarified harvest, Lane 3 shows the tangential flow filtration (TFF) product pool, Lane 4 shows the Toyopearl NH2 Product Pool, and Lane 5 shows the HIC Capto Phenyl Product Pool.

FIG. 18 shows SEC of HIC Pool Superdex 200 SEC and shows a main trimer peak with a small high molecular weight peak.

FIG. 19 shows (Left) a NS-EM raw image showing purified trimer molecules. (Right) 33938 images were classified into 32 classes and results are shown with all classes indicating well-formed trimer.

FIG. 20 shows antigenicity characterization of GT2 Capto Phenyl product pool by BLI and shows binding of purified Trimer-1 to bnAbs PGT145, PGT121, PGT128, VRC01, and BG18_GLO but not to the non-neutralizing antibodies B6 (CD4 bs) and 4025 (HIV Env antibody).

FIG. 21A shows one embodiment of a projected process for GMP production of HIV-1 Env SOSIP trimers. FIG. 21B shows one embodiment of a process for GMP production of HIV-1 Envelope trimers.

FIG. 22A shows a purification method utilizing multi-modal Capto Core 700 chromatography and anion exchange (AEX) Toyopearl NH2 750F chromatography. FIG. 22B shows a non-limiting embodiment of a process for GMP production of a nanoparticle.

FIG. 23 shows non-reduced and reduced SDS-PAGE of Process Steps for NP-2 nanoparticle purification. FIG. 23 shows a significant increase in product purity of the NH2 Product Pool (Lane 4 and 9) over the starting harvest material (Lane 1 and Lane 6). Lanes 1˜4 shows clarified harvest starting material (Lane 1), TFF product pool (Lane 2), CC700 product pool (Lane 3), and NH2 750F product pool (Lane 4) under non-reducing conditions, Lane 5 shows the molecular weight standards, and Lanes 6-9 shows clarified harvest starting material (Lane 6), TFF product pool (Lane 7), CC700 product pool (Lane 8), and NH2 750F product pool (Lane 9) under reducing conditions. The Precision Plus Protein Standard ladder from BioRad can be seen to the right of the gel.

FIG. 24 shows Superose 6 SEC UVA280 chromatogram results of the CC700 FT and NH2 Pools for purification of NP-2 nanoparticles. CC700 FT in in the blue trace shows high removal of low molecular weight impurities and the NH2 product pool in the orange trace shows significant decrease in high molecular weight impurities and further reduction of low molecular weight impurities.

FIG. 25 shows a raw NS-EM Image (Left) and 2D Class Averaging (Right). 8415 particles were imaged and 2D averaging was performed to classify these NP-2 into 32 classes. All classes show an expected profile with a ferritin shell with multiple envelope proteins attached.

FIG. 26 shows NP-2 characterization by ELISA. Antigenicity characterization results of NH2 pool by ELISA are shown. NH2 product pool exhibit the expected binding profile to key bnAb's PGT145, PGT121, BG18-GL0, and VRC01, and non-neutralizing antibodies 4025 (HIV Env antibody) and B6 (CD4 binding site non-neutralizing antibody).

FIG. 27 shows non-reduced and reduced SDS-PAGE of process steps for NP-CONS purification. FIG. 27 shows a significant increase in product purity of the NH2 Product Pool (Lane 4 and 9) over the starting harvest material (Lane 1 and Lane 6). Lanes 1˜4 shows clarified harvest starting material (Lane 1), TFF product pool (Lane 2), CC700 product pool (Lane 3), and NH2 750F product pool (Lane 4) under non-reducing conditions, Lane 5 shows molecular weight standards, and Lanes 6-9 shows clarified harvest starting material (Lane 6), TFF product pool (Lane 7), CC700 product pool (Lane 8), and NH2 750F product pool (Lane 9) under reducing conditions. The Precision Plus Protein Standard ladder from BioRad can be seen to the right of the gel.

FIG. 28 shows Superose 6 SEC UVA280 chromatogram result of NH2 Pool for NP-CONS is shown. The UV A280 trace shows high molecular weight impurities and low molecular weight impurities have been significantly reduced.

FIG. 29 shows a raw NS-EM Image (Left) and 2D Class Averaging (Right) for the NH2 product pool of NP-CONS. 3899 particles were imaged and 2D averaging was performed to classify these NP into 32 classes. The majority of the classes show an expected profile with a ferritin shell with multiple envelope proteins attached, only one class (red box) appears as incomplete nanoparticle.

FIG. 30 shows antigenicity characterization of NP-CONS NH2 product pool by Biolayer Interferometry (BLI) and shows expected binding profile to key bnAbs PGT151, PGT145, PGT128, PGT125, 2G12, VRC01, and CH106 and to non-neutralizing antibodies 17B, 19B, F393F, CH58, A32, and F105.

FIG. 31 shows Con-S sequences (SEQ ID NO: 1-47). Table 13 shows correspondence between HIV names and envelopes. Amino acid sequences in FIGS. 31 and 32 include signal peptide which is proteolytically removed during processing, including during recombinant protein production.

FIG. 32 shows the sequence of envelope HV1301189 (CH505TF.6R.SOSIP.664.v4.1 (CH505 TF4.1)) (SEQ ID NO: 48). Amino acid sequences in FIGS. 31 and 32 include signal peptide which is proteolytically removed during processing, including during recombinant protein production.

FIG. 33 shows antigenicity characterization of 50 L CHO cell culture scale (Demo Lot P035), HEK-293F affinity purified CH505 TF4.1 and small scale (≤2 L CHO cell culture) development lot P029 by Biolayer Interferometry (BLI). The data shows comparable purified product upon scale up and expected binding profile of binding to bnAbs PGT151, PGT145, 2G12, CH106 and vRC01 but not to non-neutralzing Abs 17B (CCR5 binding site), 19B (V3 loop), F39F, CH58, A32 or F105 for both affinity and non-affinity lots.

### DETAILED DESCRIPTION

A major advance in HIV-1 envelope expression has been the use of stabilized trimers, e.g. by disulfide linkages or other suitable stabilizing mutations. SOSIP trimers are some of the non-limiting examples of trimer designs. SOSIP trimers induce difficult to induce potent (tier 2) autologous HIV-1 neutralizing antibodies and weaker and sporadic heterologous neutralizing antibodies in rabbits and macaques. Expression of trimers as ferritin nanoparticle multimers has improved trimer immunogenicity. Purification of SOSIP trimers or multimers to date has been by use of either lectin columns or monoclonal antibody affinity chromatography using broadly neutralizing antibodies (bnAbs) that bind to well-folded trimers. Here we report the development of methods for purification of SOSIP trimers and for trimers in nanoparticles that preserves the purity and antigenicity of the trimer preparations that are comparable to use of affinity chromatography techniques. The strategy of trimer and nanoparticle purification reported here will enable rapid scale-up for production for human clinical trials of trimeric and multimeric HIV-1 vaccine candidates.

One approach to delivering the multimerized envelope (Env) to the immune system is by fusing Env protein to self-assembling structures, for example but not limited to proteins that form nanoparticles (He et al. Nature Communications volume 7, Article number: 12041 (2016)).

Ferritin is one of many self-assembling nanoparticles (NP) that are currently being evaluated in pre-clinical testing. These ferritin NPs are typically covalently linked via the C-terminus of the Env derivative to the N-terminus of ferritin, sometimes with a linker. The ferritin NP is comprised of 24 ferritin units, each of which is linked to an Env derivative. Fully assembled Env linked ferritin NPs designed with gp140 trimers will express 8 trimers per NP (9).

Env protein, or Env subunits, has generally been very difficult to produce and purify due to their unique characteristics. Env is heavily glycosylated and >50% of the molecular mass is comprised of glycans (10). The HIV-1 virus uses the glycans as a shield to protect the primary, conserved amino acids. Also, the glycan shield prevents proven charge based separation methods due to limited differentiation of positively or negatively charge surface exposed amino acids. Stability of the molecule is dependent on covalent and non-covalent bonding and correct assembly post-translation and processing. Incorrect intermolecular bonds and/or improper assembly during recombinant production results in contaminating product variants. (11, 12). Moreover, aberrant glycoprotein increases susceptibility to proteolytic cleavage and aggregation (13, 14).

The complexity of production and purification increases with the Env trimer and other multimer, e.g. but not limited to NP, vaccine designs. It has been shown that when expressing Env ferritin NP in cell culture, product-related impurities are expressed; these product-related impurities can include low molecular weight impurities, such as free trimers and partially assembled nanoparticles, and product-related aggregates (9).

There is a global need to develop a non-affinity, scalable process to purify trimeric HIV-1 envelopes, including trimeric envelopes as nanoparticles, for example but not limited to envelopes linked to ferritin nanoparticles. Generally, the development of Env NPs has focused on identifying and designing Env, or Env subunits, that are linked to the ferritin nanoparticle, expressed in cell culture and purified via lectin affinity chromatography or antibody affinity chromatography, and may be coupled with size exclusion chromatography (8,9). The advantages of using lectin or antibody affinity chromatography is the high specificity for Env and proven experience for affinity chromatography in a research setting.

However, custom designed antibody and lectin coupled resins have certain disadvantages (15). For example, these are prohibitively expensive and are not suitable for large scale, or worldwide vaccine production quantity. In addition to the scaling concerns, antibody coupled resins likely leach monoclonal antibody into the product and further purification is necessary to remove the leached ligand (16).

In certain aspects, the invention provides non-affinity purification methods wherein the recombinant molecule purified by the inventive methods have particular characteristics, such as specific antigenicity and structural appearance. In some embodiments, these characteristics describe a well-folded molecule, e.g. a trimer or nanoparticle. See e.g. Sanders R W, Derking R, Cupo A, Julien J P, Yasmeen A, de Val N, et al. A next-generation cleaved, soluble HIV-1 Env trimer, BG505 SOSIP.664 gp140, expresses multiple epitopes for broadly neutralizing but not non-neutralizing antibodies, PLoS pathogens. 2013; 9(9):e1003618; Dey A K, Cupo A, Ozorowski G, Sharma V K, Behrens A J, Go E P, et al. cGMP production and analysis of BG505 SOSIP.664, an extensively glycosylated, trimeric HIV-1 envelope glycoprotein vaccine candidate, Biotechnol Bioeng. 2018; 115(4):885-99, the contents of each of which are hereby incorporated by reference.

Chromatography methods used in commercial processes, including mixed-mode chromatography and ion exchange, exploit differences in physicochemical properties between the target product and contaminants.

Examples of methods for envelope purification, nanoparticle, or trimer purification, that include an affinity step in the purification are known. See e.g. He et al., 2016 “Presenting native-like trimeric HIV-1 antigens with self-assembling nanoparticles,” Nature Communications, 7:12041; eOD-GT8 purification: US Patent Publication 20180194809 and www.vaccineenterprise.org/sites/default/files/6.2%20Tsvetnitsky.pdf; US Patent Publication 20170233441; US Patent Publication 20160317460; US Patent Publication 20180194811; Verkerke H P, Williams J A, Guttman M, Simonich C A, Liang Y, Filipavicius M, Hu S-L, Overbaugh J, Lee K K. 2016, Epitope-independent purification of native-like envelope trimers from diverse HIV-1 isolates, J. Virol 90:9471-9482.

In a ferritin Env NP, each Env will impart a charge to the overall molecule. The differences in the number of Envs linked to a NP can be exploited to separate free Env, partially assembled NP, and aggregates from fully assembled NP. There is also a large difference in the size of the fully assembled NP and free Env, 30-40 nM vs 12-14 nm respectively (8,17), therefore the size difference may be utilized to separate free Env from fully assembled NP.

In one aspect, the invention provides a process for isolating/purifying HIV-1 envelopes, including trimers and NPs comprising envelopes, using methods and materials suitable for industrial scale and current Good Manufacturing Practices. In certain embodiments, the purification methods do not include any affinity chromatography steps. Custom resins with coupled lectin or antibodies are not considered suitable due to the limitations of affinity coupled resins and cost.

HIV-1 Envelopes as Trimers and Nanoparticles (NPs)

Any HIV-1 envelope expressed recombinantly could be purified by the methods of the invention. Envelope proteins designed to multimerize, e.g. but not limited to a trimer and/or nanoparticle could be purified by the instant methods. Non-limiting embodiments of trimer designs include any SOSIP design, native flexibly linked (NFL) trimer designs, uncleaved prefusion-optimized (UFO) trimer designs, or any other trimer design. See e.g. He et al. Science Advances 21 Nov. 2018: Vol. 4, no. 11, eaau6769 DOI: 10.1126/sciadv.aau6769 and references therein.

In certain embodiments, the nanoparticle is a molecule comprising stabilized native-like HIV-1 Envelope gp140 trimer. In some embodiments, the envelope is engineered to bind to antibody precursors. In some embodiments, the design allows incorporation into a self-assembling nanoparticle, for example but not limited to a ferritin nanoparticle. Ferritin protein self assembles into a small nanoparticle with three fold axis of symmetry. At these axis the envelope protein is fused. Therefore the assembly of the three-fold axis also clusters three HIV-1 envelope protomers together to form an envelope trimer. Each ferritin particle has 8 axises which equates to 8 trimers being displayed per particle. See e.g. Sliepen et al., Retrovirology, 2015 12:82, DOI: 10.1186/s12977-015-0210-4. Any suitable ferritin sequence could be used. A non-limiting embodiment of NP comprises Helicobacter pylori ferritin sequence. In non-limiting embodiments, the NP is based on single gene construct that contains envelope sequence that forms a trimer, followed by a linker sequence, followed by the sequence of ferritin from the hyperthermophilic archaeal anaerobe Pyrococcus furiosus. The design of this nanoparticle is intended to produce particles with 24 copies of the fusion protein that exposes 8 trimers on the particle exterior, based on the fact that P. furiosus ferritin expressed alone assembles into nanoparticles with 24 copies of the ferritin protein, in which the N-terminus of each ferritin is exposed on the exterior of the particle. The expected molecular weight of a fully assembled −NP is 2.2 MDa and the diameter of the NP is expected to be 30 nm from the end of one gp140 trimer to its opposite gp140 trimer on a given symmetry axis. In non-limiting embodiments, the ferritin nanoparticle is formed via sortase A reaction. See e.g. Tsukiji, S. and Nagamune, T. (2009), Sortase-Mediated Ligation: A Gift from Gram-Positive Bacteria to Protein Engineering. ChemBioChem, 10: 787-798. doi:10.1002/cbic.200800724; Proft, T. Sortase-mediated protein ligation: an emerging biotechnology tool for protein modification and immobilisation. Biotechnol Lett (2010) 32: 1. doi:10.1007/s10529-009-0116-0; Lena Schmohl, Dirk Schwarzer, Sortase-mediated ligations for the site-specific modification of proteins, Current Opinion in Chemical Biology, Volume 22, October 2014, Pages 122-128, ISSN 1367-5931, dx.doi.org/10.1016/j.cbpa.2014.09.020; Tabata et al. Anticancer Res. 2015 August; 35(8):4411-7; Pritz et al. J. Org. Chem. 2007, 72, 3909-3912.

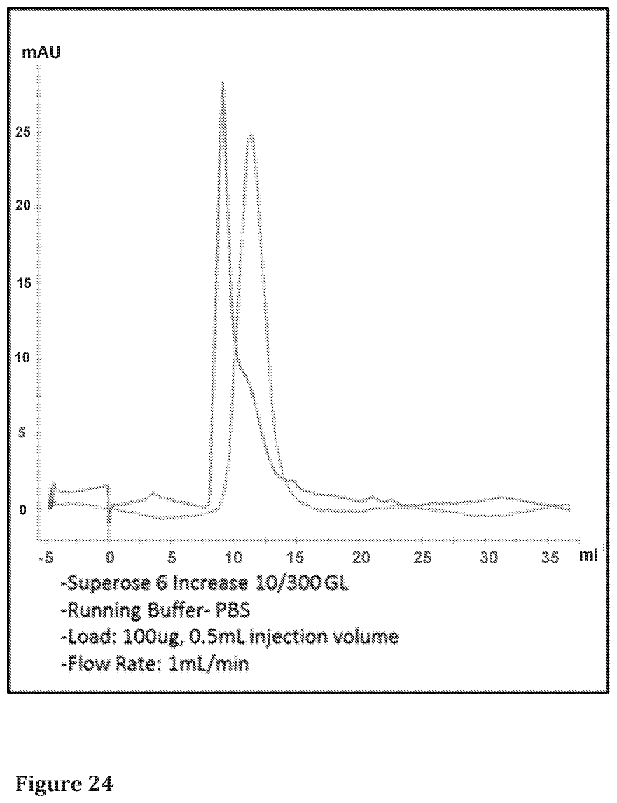

Non-limiting examples of nanoparticles and trimers contemplated for purification by the inventive methods are disclosed in WO2017151801 (CH505 based envelopes, see without limitation Table 1, FIG. 24), PCT/US2017/020823 published as WO2017152146 (CH848 based envelopes), and PCT/US2018/020788 published as WO2018161049 (CH848 based envelopes), WO/2018/218225, PCT/US2019/049431 filed Sep. 4, 2019 (19CV3 designs), PCT/US2019/049662 filed Sep. 5, 2018 (trimer stabilizing designs), WO/2019/169356, the contents of each of which are incorporated by reference in their entirety.

In some embodiments, the eOD-GT8 60-mer nanoparticle (see US Patent Publication 20180194809) is purified by the methods of the present invention.

Resins and Chromatography Conditions

Capto Core 700 is a GMP compliant, scalable multimodal resin produced by GE Life Sciences. The base matrix for this resin is highly cross-linked agarose that is base stable for easy cleanability and can withstand high pressure that enables decreased run times. This resin exhibits multimodal separation modalities with separation occurring by size, ionic properties, and hydrophobicity. This does have a mix-mode component, as the ligand on the inside of the bead is mix-mode (ion exchange and hydrophobic interaction). The Capto Core 700 is composed of a ligand-activated core and inactive shell; the inactivated shell excludes molecules that are larger than the apparent size of a globular 700 kD protein. Molecules smaller than a 700 kD globular protein can enter the pore and bind the mix-mode ligand-activated core, the active ligand is an octlyamine that exhibits both hydrophobic and anion exchange modes of interaction. HIV envelope ferritin nanoparticles have been shown to have low molecular weight product-related impurities, these can include free gp140 subunits (i.e. gp120 and/or gp41), free gp140 monomer, free gp140 trimer, and nanoparticle that is not completely assembled. Low molecular weight impurities are small enough to pass through the inactivated shell and bind the ligand core, while fully assembled nanoparticle cannot pass through the inactivated shell and thus can be collected in the column flow through fraction. The invention contemplates any other resin which has properties and characteristic that are functionally equivalent.

Possible alternatives resins can include other Capto Core series of resins that implement the core bead technology, including the Capto Core 400. Other alternatives to the Capto Core 700 are tangential flow filtration (hollow fiber and cassettes) with molecular weight cutoffs >300 kD that can separate low molecular weight impurities based on size differences. These can include, but are not limited to, Pellicon 2 300 kD, 500 kD, and 1000 kD produced by Millipore Sigma; mPES 300 kD, 500 kD, 700 kD hollow fibers, and PS 500 kD hollow fibers produced by Spectrum Labs (a Repligen brand), and equivalent filters from other manufacturers.

Anion exchange chromatography (AEX) is a known process used in protein purification protocols. AEX separates substances based on their charge using ion exchange resin. AEX conditions of operation, such as resins, buffers (e.g. but not limited to salt, pH), flow rates, etc. are experimentally determined by routine optimization using well known principles of operating AEX chromatography and the nature of the purified protein.

In non-limiting embodiments, AEX purification is the first chromatographic step in the methods of the invention. A skilled artisan can readily determine the conditions of operations of the AEX step. In non-limiting embodiments, 20 L-30 L or 20-25 L of harvest pool material is loaded on the column per one liter of resin. In some embodiments, less than 30 L of harvest pool material is loaded per one liter of resin. In some embodiments, at least 20 L, 21 L, 22 L, 23 L, 24 L, 25 L, 26 L, 27 L, 28 L, 29 L, or 30 L of harvest pool material is loaded per one liter of resin. In some embodiments, 20 L, 21 L, 22 L, 23 L, 24 L, 25 L, 26 L, 27 L, 28 L, 29 L, or 30 L of harvest pool material is loaded per one liter of resin. The harvest pool material could be subjected to any additional treatment steps, including without limitation, clarification, TFF, viral clearance, and so forth.

In non-limiting embodiments, the equilibration/wash buffer is at pH 7-7.4. In non-limiting embodiments, the equilibration/wash buffer has a conductivity of 24-31 mS/cm. In non-limiting embodiments, the elution buffer is pH 7-7.4. In non-limiting embodiments, the elution buffer has a conductivity of 55-68 mS/cm. The elution buffer is high salt, e.g. 0.5M-0.6M salt, where the specific concentration is experimentally determined.

Toyopearl NH₂ 750F is a GMP compliant, scalable chromatography resin produced by Tosoh Biosciences. Toyopearl NH2 750F resin is the same as Tosoh NH2 750F resin. The base matrix of this resin is a hydroxylated methacrylic polymer beads that is base stable for easy cleanability and demonstrates good pressure-flow characteristics. The Toyopearl NH2 750F base bead has been functionalized with a primary amine active ligand, resulting in anion-exchange mode of interaction. Negatively charged molecules can bind the active ligand and can be selectively eluted via changes in pH, ion concentration, and/or counter ion. The invention contemplates any other resin which has properties and characteristic that are functionally equivalent.

Alternative resins may include anion exchange resins, membranes, or monoliths that utilize the following active ligands or other ligands that operate in anion exchange mode, with or without a spacer: quaternary amine (Q), diethylaminoethyl (DEAE), diethylaminopropyl, primary amine. Alternative resins may include cation exchange resins, membranes, or monoliths that utilize the following active ligands or other ligands that operate in cation exchange mode, with or without a spacer: sulfonate (S), sulfopropyl (SP), carboxymethyl (CM), sulfate. Alternative resins may include mix-mode resins in which one of the modes of binding is ion exchange and can include any of the ligands listed for alternate anion exchange or cation exchange resins, membranes, or monoliths.

Alternative resins may include anion exchange resins, membranes, or monoliths that utilize a positively charged functional group with or without a spacer. Example alternative, anion exchange resins include but are not limited to the following: Capto Q (GE Healthcare), Capto Q XP (GE Healthcare), Eshmuno Q (EMD Millipore), Fractogel TMAE (EMD Millipore), POROS XQ (Thermo Fisher Scientific), POROS HQ (Thermo Fisher Scientific), POROS PI (Thermo Fisher Scientific), Toyopearl GigaCap Q-650M (Tosoh Biosciences), and Toyopearl SuperQ-650M (Tosoh Biosciences).

Capto Phenyl/Phenyl HIC:

Hydrophobic Interaction Chromatography (HIC) is a well known process for protein purification based on interaction between hydrophobic regions in the protein of interest and the HIC media. Specific chromatographic conditions are readily determined by routine optimization of HIC conditions, including HIC media, and conditions of operations. In some embodiments, the hydrophobic chromatography operation can be operated in flow through mode and/or bind and elute mode. A skilled artisan can modify loading conditions to optimize the operation for the preferred method. A non-limiting example range of loading salt concentration for execution of the hydrophobic step may include 0.4M-2M, with flow through mode incorporating lower salt concentration within the range and bind and elute incorporating higher salt concentration within the range.

In certain embodiments, the HIC step is a polishing chromatography step using Capto Phenyl resin. In certain embodiments, the Capto Phenyl polishing step is operated in flow through mode and removes host cell contaminants and product-related impurities. This resin has a phenyl ligand hydrophobic mechanism of action. In certain embodiments, the loading fraction from the previous process step, e.g. nanofiltrate, NH2 eluate from an AEX step, or a CHT eluate, is spiked to a final concentration 0.6M ammonium sulfate with 20 mM HEPES, 1.2M ammonium sulfate, pH 7.0-7.4 (i.e., pH 7.0, pH 7.1, pH 7.2, pH 7.3, or pH 7.4). The Capto Phenyl column is sanitized, equilibrated, loaded, and washed. The load flow through and initial wash is combined into a single product fraction. The column is then stripped, sanitized, and stored.

In some embodiments, the polishing HIC step could follow the CHT step. In these embodiments a viral filtration step follows the HIC step. In some embodiments, a viral filtration step is incorporated between the CHT and polishing HIC step.

Alternative resins may include hydrophobic interaction resins that utilize a hydrophobic functional group, such as phenyl, butyl, octyl, polypropylene glycol or other ligands that utilizes hydrophobic interactions, with or without a spacer. Example alternative, hydrophobic interaction resins include but are not limited to the following: Capto Octyl (GE Healthcare), Capto Butyl (GE Healthcare), Capto Phenyl ImpRes (GE Healthcare), Toyopearl Phenyl-650M (Tosoh Biosciences), Toyopearl Butyl-650M (Tosoh Biosciences), Toyopearl PPG-600M (Tosoh Biosciences).

In some embodiments, the hydrophobic interaction chromatography operation can be operated in flow through mode and/or bind and elute mode. A skilled artisan can modify loading conditions to optimize the operation for the desired mode. A non-limiting example range of loading salt concentration for execution of the hydrophobic interaction chromatography step may include 0.4M-2M, with flow through mode incorporating salt concentration within the lower end of the range, and bind and elute incorporating higher salt concentration within the range. In non-limiting embodiments, flow through operation is carried out at 0.4M, 0.5M. 0.6M, 0.7M, 0.8M, 0.9M, 1.0M, 1.1M, 1.2M, 1.3M salt concentration. In non-limiting embodiments, bind and elute operation is carried out at 1.0M, 1.1M, 1.2M, 1.3M, 1.4M, 1.5M, 1.6M, 1.7M, 1.8M, 1.9M, 2.0M salt concentration.

Any other suitable chromatographic step or resin is contemplated by the purification methods of the invention. In certain embodiments, antibody affinity or lectin affinity resins and steps are excluded from the methods of the invention.

Various buffers are used in the purification steps of the invention. Variations in the buffers or any other suitable buffer system could be used in the purification steps of the invention. A skilled artisan can readily determine buffer variations that would be suitable. A skilled artisan can readily determine specific variation in buffer and chromatography operation conditions for specific envelope trimers and/or nanoparticles.

Another chromatography step (intermediate) uses ceramic hydroxyapatite resin to bind and elute the target molecule into a single bulk fraction. This resin has calcium affinity interaction and cation exchange interaction mechanisms of action. This step removes host cell contaminants and product-related impurities. The CHT column is charged, equilibrated, loaded, washed, and product fraction is eluted. The column is then stripped, sanitized, and stored. In certain embodiments, the CHT column is operated in downflow. Conditions for CHT operation are readily determined by routine optimization using well known principles of CHT chromatography and the nature of the purified protein. In non-limiting embodiments, the elution buffer has pH 7-7.4. In non-limiting embodiments, the elution buffer has conductivity of 3-5 mS/cm.

The present invention relates to methods of purifying HIV-1 Env from a recombinant cell culture, including but not limited to liquid harvested from cell culture (e.g. but not limited, human embryonic kidney (HEK) cell culture, chinese hamster ovary (CHO) cell culture). The liquid is typically clarified from cellular debris by depth filtration and/or centrifugation.

The purification methods described herein could be used on material produced from stable cell lines or transiently transfected cell lines.

In certain embodiments, the first step in the process is tangential flow filtration (TFF) (ultrafiltration and diafiltration) to prepare the clarified harvest for capture load and to control processing volume. Any suitable MW cut off can be used. In a non-limiting embodiments, using a 300 kD nominal MW cutoff, the tangential flow filtration is also a critical purification step used to remove small host cell proteins. The TFF step leverages molecule size to remove cell culture waste products and concentrate/diafilter product.

In a non-limiting embodiment, the TFF filter is assembled, flushed with purified water and flushed with 20 mM HEPES, 250 mM NaCl, pH 7.2 (DF Buffer). The clarified harvest is then concentrated 5-7×, followed by diafiltration with 5 diavolumes of DF buffer. The product is recirculated and recovered, then the membrane is flushed with DF buffer to increase the step yield.

In certain embodiments, the methods of the invention comprise an additional step(s) to reduce viral load. In a non-limiting embodiment, the step is a dedicated viral removal step. In a non-limiting embodiment, the step is nanofiltration. In some non-limiting embodiments, nanofiltration is carried out with the Viresolve Pro (Vpro) nanofilter. The nanofilter has a nominal exclusion limit of 20 nm and virus removal is achieved via size partitioning. The nanofilter is protected by placing the Viresolve Shield H guard filter upstream of the Vpro. The fraction from the previous step, e.g. elute or flow through, is nanofiltered and a buffer flush is performed. The combined filtered fraction and flush is the nanofiltrate. In some embodiments, the Vpro assembly has an upstream Shield H (with 1:1 filter area ratio of Shield H to Vpro) to act as a guard filter for the nanofilter. In a non-limiting embodiment, the assembly is flushed with purified water and suitable buffer, e.g but not limited to elution buffer. The fraction is then filtered at a pressure of 25-35 psi (i.e., 25, 26, 27, 28, 29, 30, 31, 32, 33, 34, or 35 psi) and followed by a buffer flush to recover product from the filter housing.

In certain embodiments, the methods of the invention comprise an additional step of UFDF operation. In certain embodiment, the UFDF step is performed to concentrate and buffer exchange product into the final formulation buffer.

In a non-limiting embodiments, the UFDF filter is assembled, flushed with purified water and flushed with 20 mM Tris, 100 mM NaCl, pH 7.5 (formulation buffer). The product is then concentrated to a target of 1.5 g/L, followed by diafiltration with 7 diavolumes of formulation buffer. The product is recirculated and recovered, then the membrane is flushed with formulation buffer to increase the step yield. The UFDF Retentate and UFDF Flush are pooled and mixed.

In non-limiting embodiments, all steps in the culture (upstream) and purification (downstream) methods are readily scaled for large volume of industrial scale production, e.g. but not limited to 50 L, 100 L, 150 L, 200 L, 250 L, 300 L, 350 L, 400 L, 450 L, 500 L, 550 L, 600 L, 650 L, 700 L, 750 L, 800 L, 850 L, 900 L, 950 L, 1,000 L, 1,500 L, 2,000 L, 2,500 L, 3,000 L, 3,500 L, 4,000 L, 4,500 L, 5,000 L, 6,000 L, 7,000 L, 8,000 L, 9,000 L, 10,000 L, 15,000 L, 20,000 L or higher bioreactor scale.

The methods of the invention utilize reagents, such as, but not limited to, filters and resins, which are readily available in formats that can support large production volumes. All resins are commercially available in lot sizes in excess of hundreds of liters per lot

### List of References

8. He et al. Nature Communications volume 7, Article number: 12041 (2016)
9. Sliepen et al. Retrovirology vol. 12, Article number: 82 (2015) 10.1186/s12977-015-0210-4
10. Go et al. JOURNAL OF VIROLOGY, August 2011, p. 8270-8284 jvi.asm.org/content/early/2011/06/08/JVI.05053-11.short
11. Go et al. J. Proteome Res. 2011, 10, 2, 578-591 pubs.acs.org/doi/ipdf/10.1021/pr100764a
12. Ringe et al. J. Virol. doi:10.1128/JVI.01768-15
13. B. Yu, D. P. A. J. Fonseca, S. M. O'Rourke, P. W. Berman, Protease Cleavage Sites in HIV-1 gp120 Recognized by Antigen Processing Enzymes Are Conserved and Located at Receptor Binding Sites. J. Virol. 84:1513 (2010),
14. Finzi et al. Journal of Virological Methods 168 (2010) 155-161.
15. Zhao et al. Vaccine Volume 37, Issue 36, 23 Aug. 2019, Pages 5491-5503
16. Dey et al. Biotechnol Bioeng. 2018 April; 115(4):885-899. doi: 10.1002/bit.26498. Epub 2017 Dec. 11.
17. Sai Prasad N. Iyer, Michael Franti, Ammie A. Krauchuk, Danielle N. Fisch, Amadou A. Ouattara, Kenneth H. Roux, Laura Krawiec, Antu K. Dey, Simon Beddows, Paul J. Maddon, John P. Moore, and William C. Olson.AIDS Research and Human Retroviruses. June 2007. ahead of print http://doi.org/10.1089/aid.2006.0261

### EXAMPLES

### Example 1—Purification of HIV-1 Envelope Trimers and Nanoparticles by Chromatography Methods that do not Require Antibody Affinity Chromatography

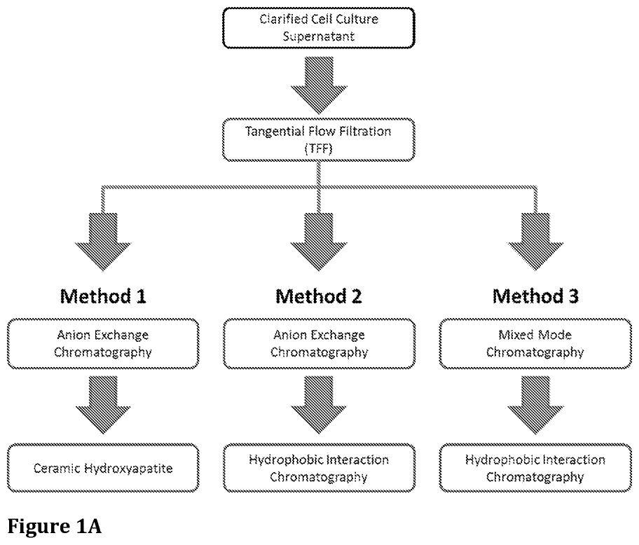

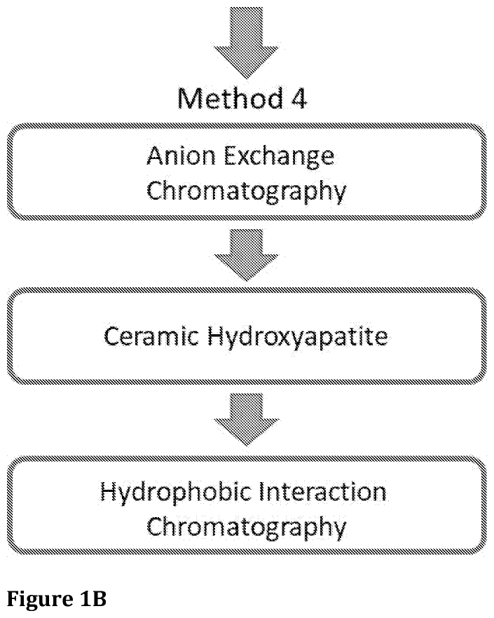

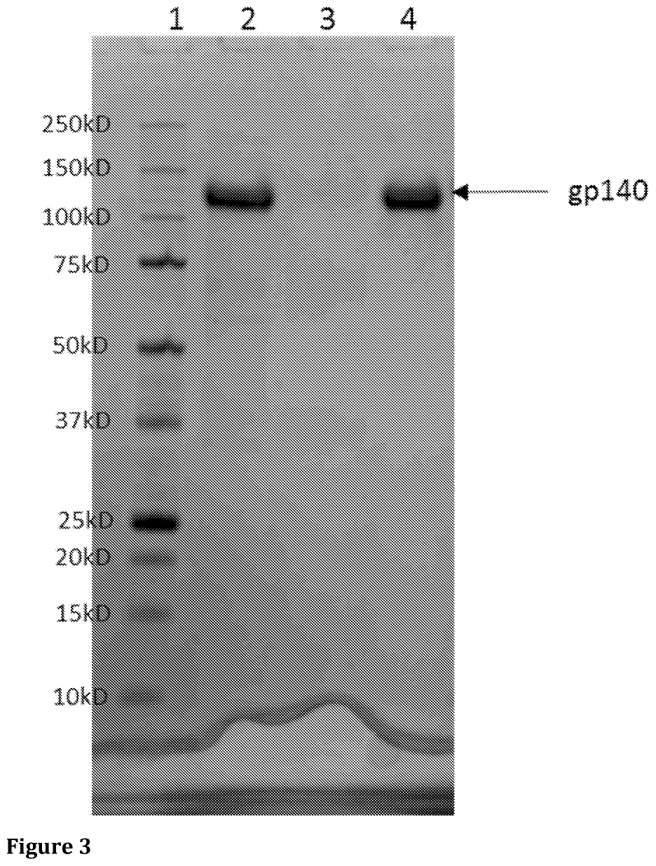

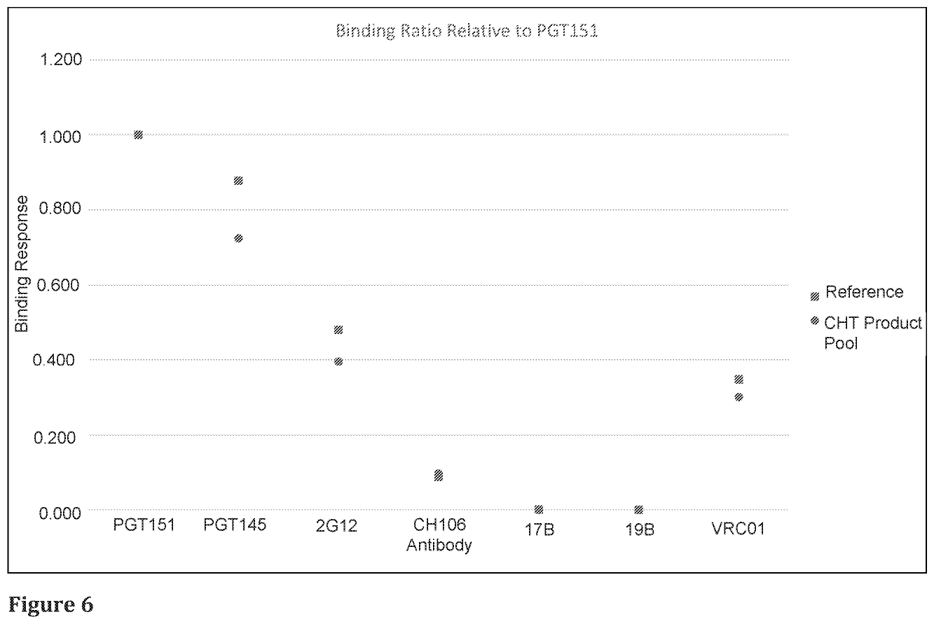

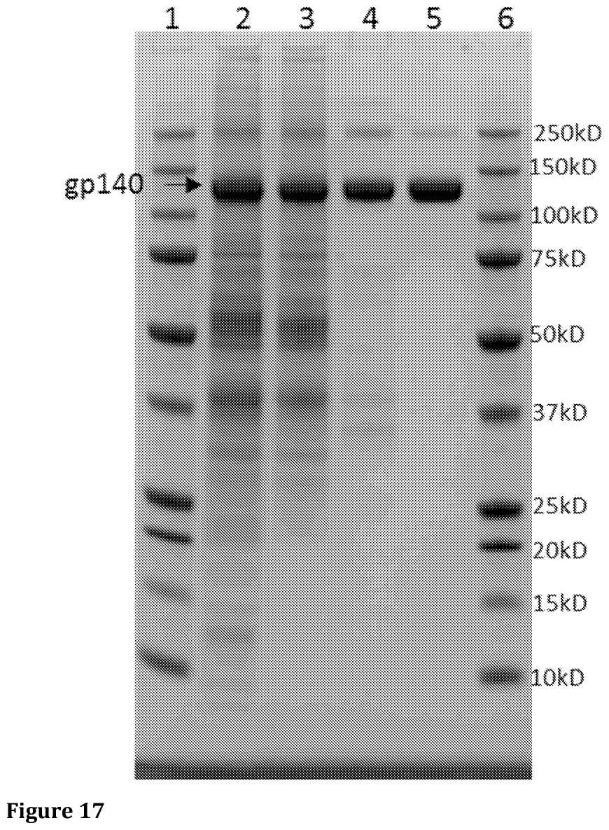

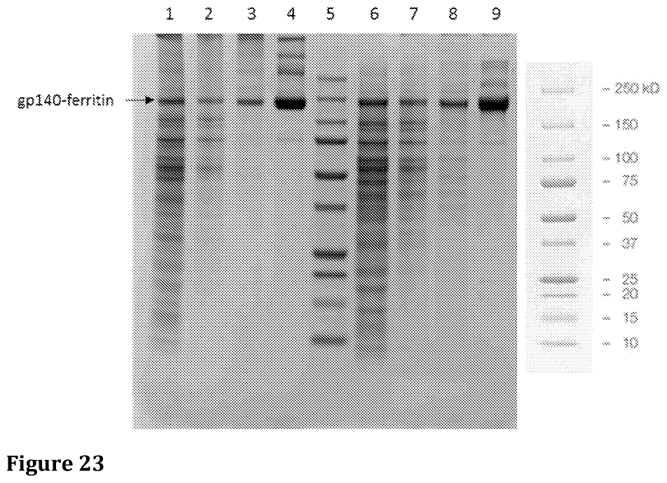

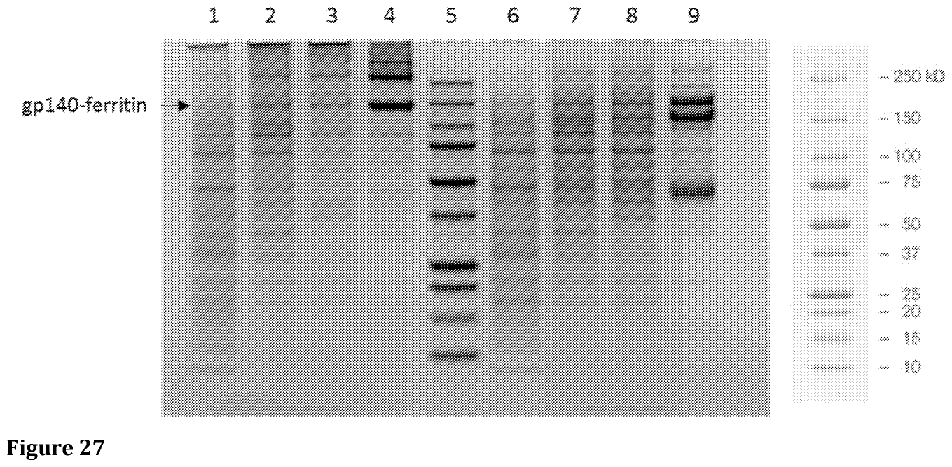

FIGS. 1-21 are directed to SOSIP trimers—FIGS. 3-6, FIGS. 8-11, and FIGS. 13-16 are directed to trimer CH505 TF stabilized SOSIP, FIGS. 17-20 are directed to Trimer 2; FIGS. 23-26 are directed to NP-2, FIGS. 27-30 are directed to NP-CONS.

The SEC and NS-EM results show that fully assembled, uniform trimers and nanoparticles were isolated and the ELISA results confirm that the purified nanoparticles exhibit expected binding characteristics to key antibodies.

Abstract

A major advance in HIV-1 envelope expression has been the use of stabilized trimers by disulfide linkages (SOSIP trimers). SOSIP trimers induce difficult to induce potent (tier 2) autologous HIV-1 neutralizing antibodies and weaker and sporadic heterologous neutralizing antibodies in rabbits and macaques. Expression of trimers as ferritin nanoparticle multimers has improved trimer immunogenicity. Purification of SOSIP trimers or multimers to date has been by use of either lectin columns or monoclonal antibody affinity chromatography using broadly neutralizing antibodies (bnAbs) that bind to well-folded trimers. Here we report the development of schema for purification of SOSIP trimers and for trimers in nanoparticles that preserves the purity and antigenicity of the trimer preparations that are comparable to use of affinity chromatography techniques. The strategy reported here of trimer and nanoparticle purification should enable rapid scale-up for production for human clinical trials of trimeric and multimeric HIV-1 vaccine candidates.

Introduction

Induction of broadly neutralizing antibodies (bnAbs) is a central goal of HIV-1 vaccine development, and the HIV-1 envelope (Env) is the sole neutralizing antibody target (1). Thus, a myriad of Env designs have been proposed to be used as immunogens for induction of protective antibodies (1). Two of the most promising recent designs of soluble HIV-1 envelopes have been the disulfide-linked SOSIP trimer (2) and SOSIP trimers in ferritin nanoparticles.

SOSIP proteins as trimers or as nanoparticles are generally purified by either lectin or monoclonal antibody affinity chromatography in order to enrich for well-folded trimers. Stability and correct folding of the Env trimer is dependent on covalent and non-covalent bonding as well as correct Env assembly post-translational processing. Incorrect patterns of the 9 gp120 intermolecular disulfide bonds and/or improper gp120 protomer assembly results in non-native Env variants (3). The current status of HIV-1 vaccine development field is to use various Env constructs to target the germline B cell receptors of bnAb B cell lineages (4, 5) and then to use sequential Envs to boost germline targeting Envs (6). Germline targeting Env forms can be gp120 core proteins (7), gp120s (5, 8), or SOSIP trimers (9). Thus, a final HIV-1 vaccine to induce bnAbs has the possibility of consisting of multiple sequential Env forms (8, 10). Moreover, the HIV-1 vaccine development field is moving to a strategy of immunizing humans in iterative phase I clinical trials to determine Env construct safety and to determine human immunogenicity. Thus it is critical to have purification protocols in place that meet target quality characteristics of Env trimer preparations, and are also scalable and easy to implement in the production of clinical grade material under current good manufacturing practices (cGMPs).

The current state-of-the-art purification method of SOSIP trimers is to use a combination of monoclonal affinity chromatography, followed by mixed-mode anion exchange (MM-AEX) Capto-adhere column and size exclusion chromatography (SEC) (11). While this method can successfully isolate and prepare preparations of well-folded SOSIP trimers, development of methods that are scalable to full commercial production and not limited by the restraints of antibody affinity and size exclusion chromatography, while maintaining high product quality would be beneficial. Moreover, with current techniques, staph protein A columns are needed to remove any contaminating monoclonal antibody that may have leached from the Ab affinity column (11). In addition, large scale downstream processes to purify multimers of SOSIP trimers such as ferritin-SOSIP trimer fusion protein in trimer octamers are needed for scale-up of trimer nanoparticles. Here we describe schema for purification of SOSIP trimers and trimer nanoparticles that do not depend on affinity or size exclusion chromatography. These methods result in Env preparations of high quality that are scalable, and can easily and rapidly be implemented for GMP production of clinical trial material.

Methods and Reagents

Toyopearl NH2 750F resin is the same as Tosoh NH2 750F resin

Antibodies—as referenced in (12).

Recombinant antibodies were used to determine the antigenicity of recombinant envelope. The panel of antibodies consisted of PGT151 is a timer-specific broadly neutralizing antibody that targets the gp120:gp41 interface of HIV-1 envelope (Broadly neutralizing HIV antibodies define a glycan-dependent epitope on the prefusion conformation of gp41 on cleaved envelope trimers, Falkowska E., Immunity, 2014, PMID:24768347). PGT145 is a trimer-specific broadly neutralizing antibody against the V2 apex epitope on HIV-1 envelope (Walker et al, Nature, 2011 Sep. 22, 477(7365):466-70, PMID: 21849977). 2G12 is an HIV-1 envelope glycan-reactive antibody that targets the N332 glycan and proximal glycans on HIV-1 envelope outer domain (Trkola et al., J Virol., 1996 February, 70(2):1100-8, PMID: 8551569). 17B is a coreceptor binding site antibody. 19B is a narrow neutralizing third variable region HIV-1 antibody (Moore et al., J Virol., 1995 January, 69(1):122-30, PMID:7527082). CH106 and VRC01 are CD4 binding site broadly neutralizing antibodies (Liao et al, Nature, 2013 Apr. 25, 496(7446):469-76, PMID: 23552890 and Wu et al, Science, 2010 Aug. 13, 329(5993):856-61, PMID: 20616233). Recombinant antibodies were produced as previously described (Saunders et al., Cell Rep., 2017 Feb. 28, 18(9):2175-2188, PMID: 28249163). 293i cells were diluted to 2.5 million cells/mL in Expi293 media on the day of transfection. 293i cells were co-transfected with 400 μg of heavy chain plasmid and 400 μg of light chain plasmid using Expifectamine per the manufacturer's protocol. Five days after transfection the cells were centrifuged and the cell culture supernatant was collected and filtered with a 0.8 μm filter. The cell-free supernatant was concentrated to approximately 50 mL total volume and incubated with protein A beads (ThermoFisher) overnight at 4° C. The protein A beads were centrifuged for 5 min at 1200 rpm in a Sorval table top centrifuge. The beads were resuspended in 25 mL of PBS with 340 mM NaCl to wash them and pipeted into an empty plastic column. The antibody was eluted off of the beads with two elutions of 15 mL each of 10 mM glycine pH 2.4 150 mM NaCl. The pH was neutralized by adding 1M Tris pH8.0 to a final volume of 10%. The eluate was concentrated in a Vivaspin 15 and buffer exchanged into PBS with successive rounds of centrifugation.

Envs and nanoparticles—as referenced (13, 14)

To cover the diversity of HIV-1 isolates that circulate globally a consensus envelope sequence was derived from all group M HIV-1 isolates available at the time termed CON-S (Liao et al Virology. 2006 Sep. 30; 353(2):268-82.PMID: 17039602). To create stable mimics of the HIV-1 Env CON-S we created SOSIP gp140s that were stabilized by introducing BG505 amino acids into the gp120 and gp41 regions and a disulfide bond between amino acids 201 and 433 (Saunders et al., Cell Rep., 2017 Dec. 26, 21(13):3681-3690, PMID: 29281818 and Kwon et al., Nat Struct Mol Biol., 2015 July, 22(7):522-31, PMID: 26098315). The CON-S sequence was further optimized to bind to antibodies that target the V3-glycan broadly neutralizing site by removing glycans that were determined in neutralization assays to inhibit V3-glycan antibody binding and neutralization. It has been shown that B cell receptors recognize low affinity antigen better when it is presented on a surface rather than free in solution (B cells extract and present immobilized antigen: implications for affinity discrimination, Batista F D, Neuberger M S, EMBO J., 2000 Feb. 15, 19(4):513-20, PMID: 10675320). Thus we fused to the C-terminus of CON-S gp140 SOSIP a ferritin subunit from H. pylori. Ferritin self assembles into a 24-mer nanoparticle which displays 8 copies of the CON-S SOSIP trimer on its surface.

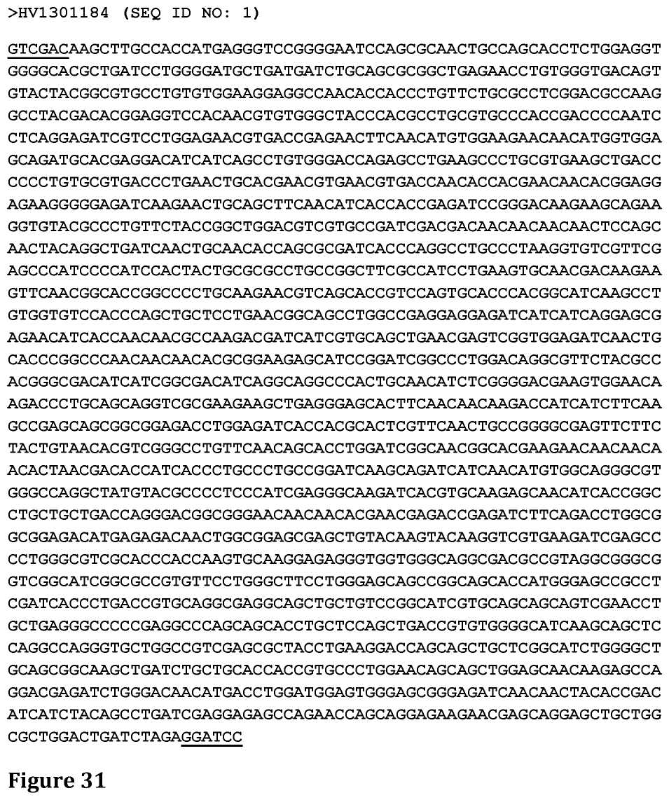

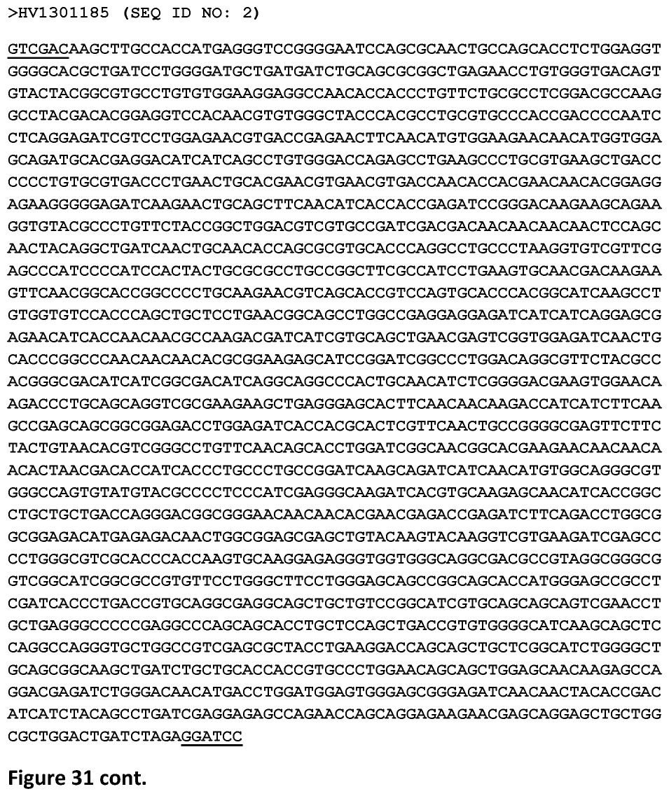

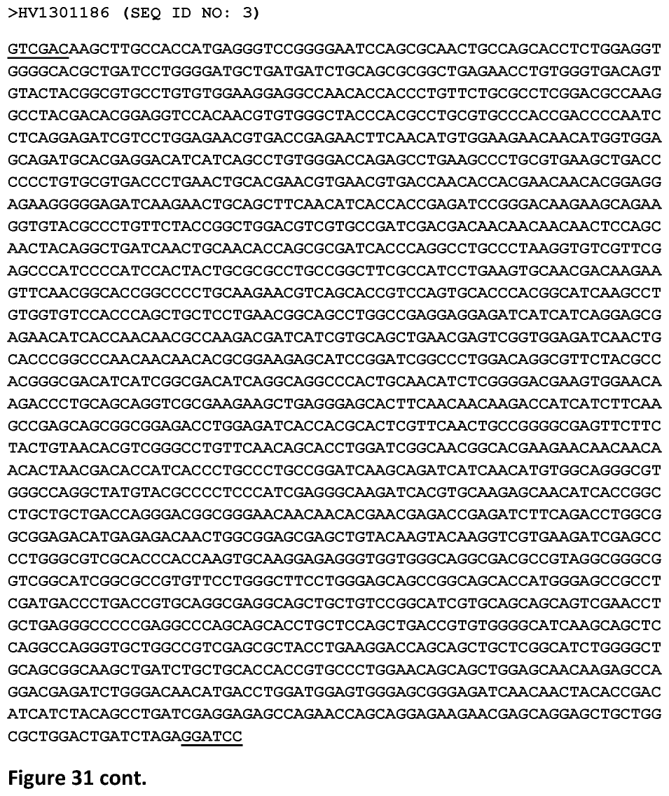

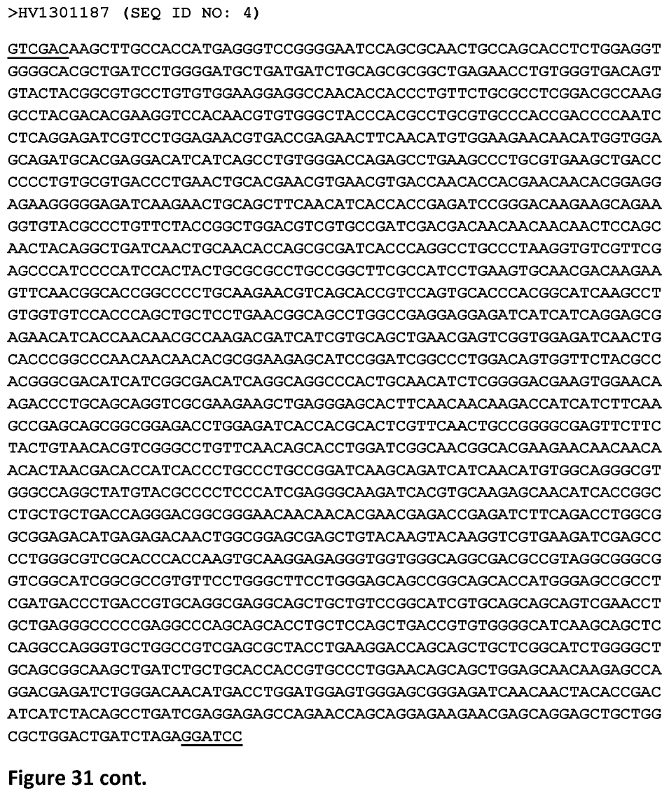

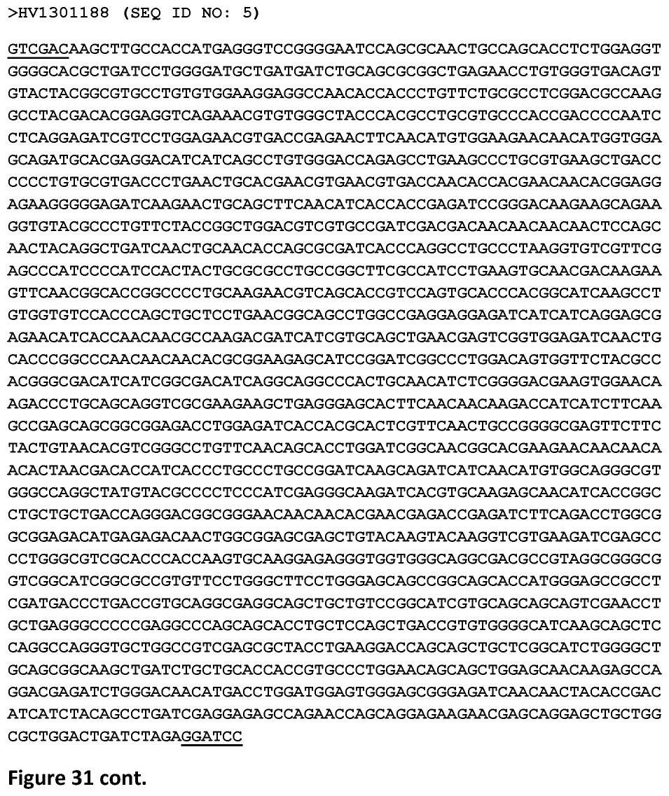

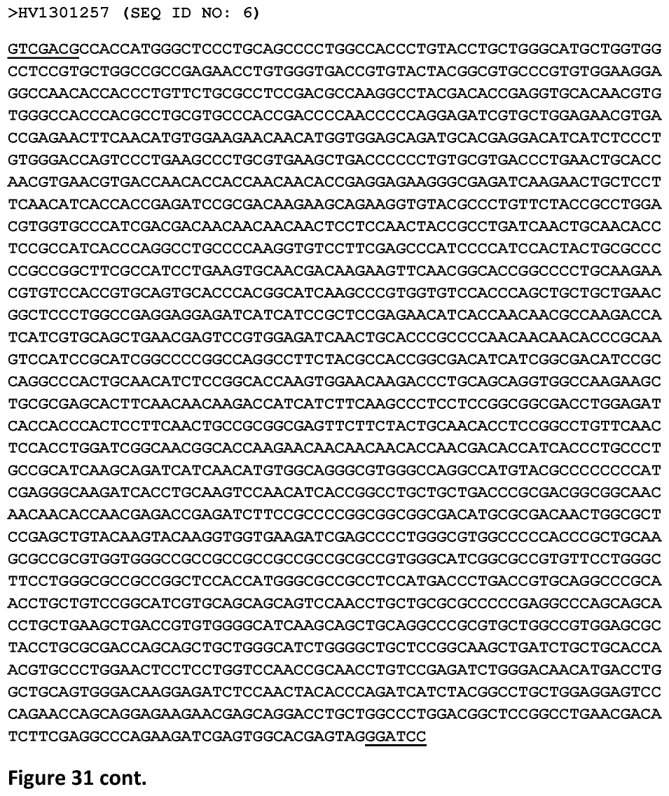

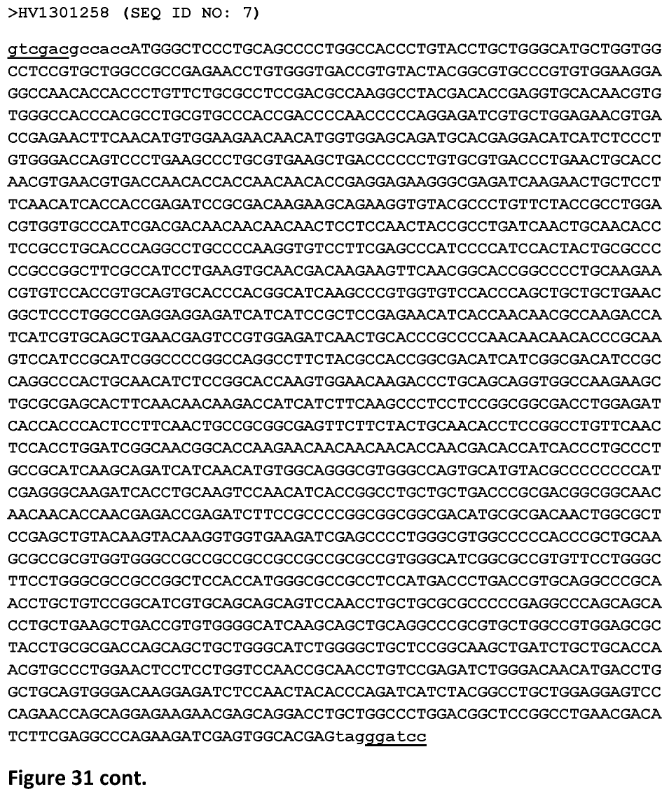

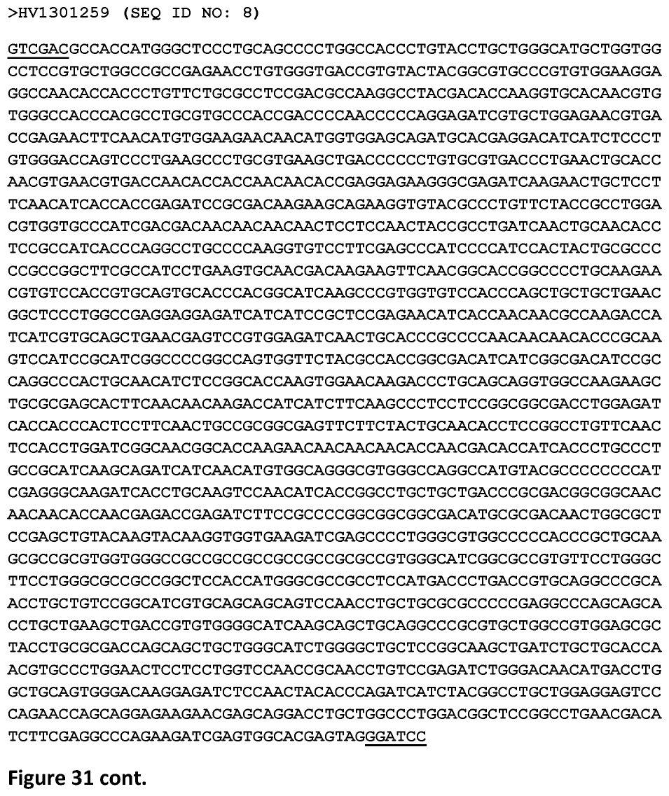

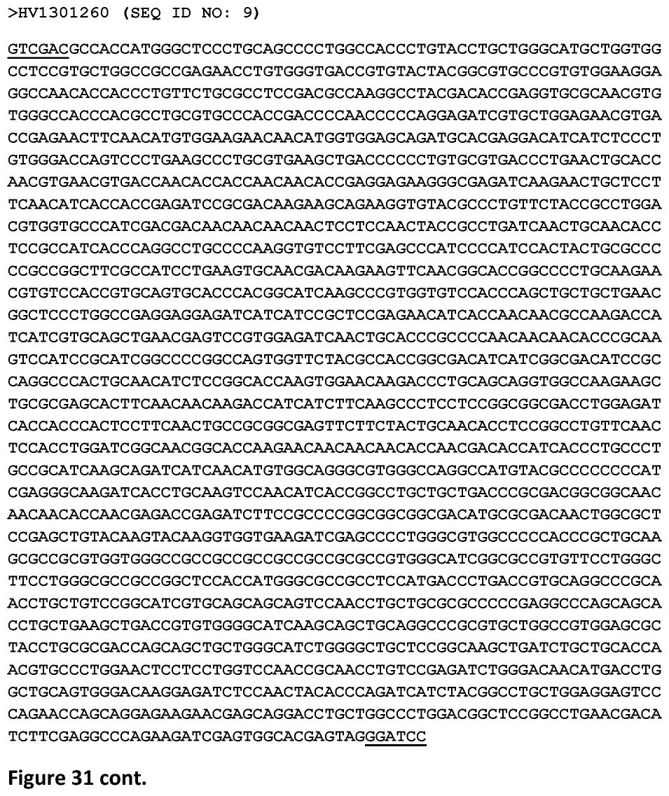

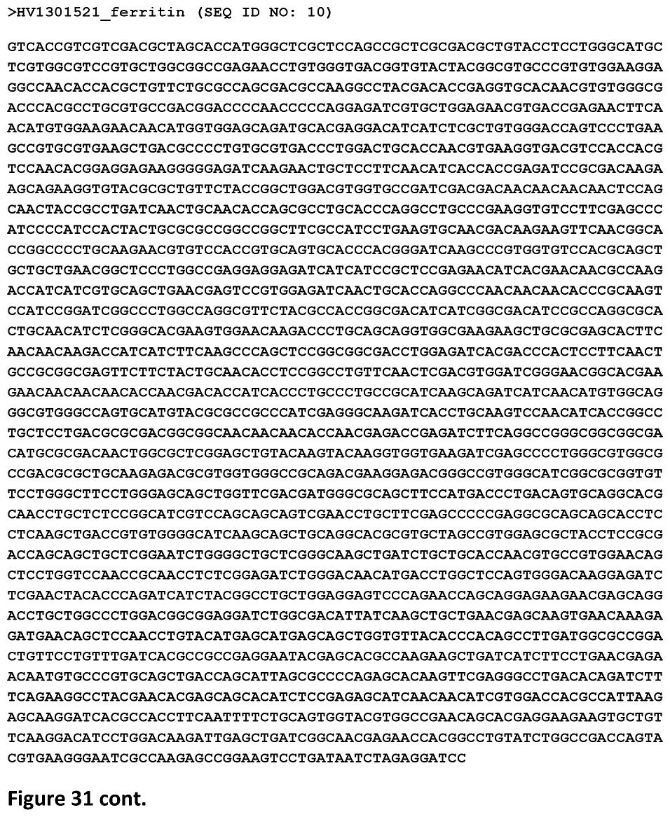

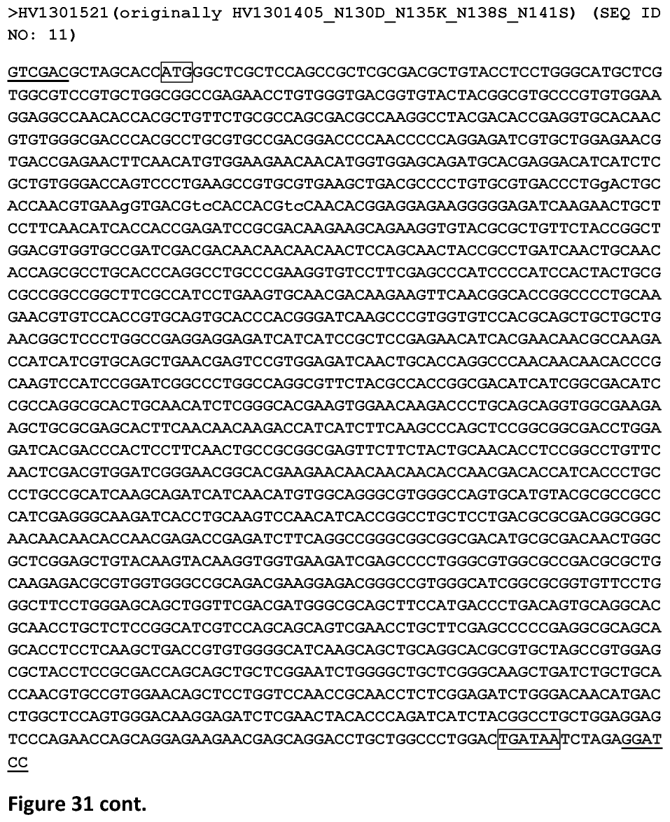

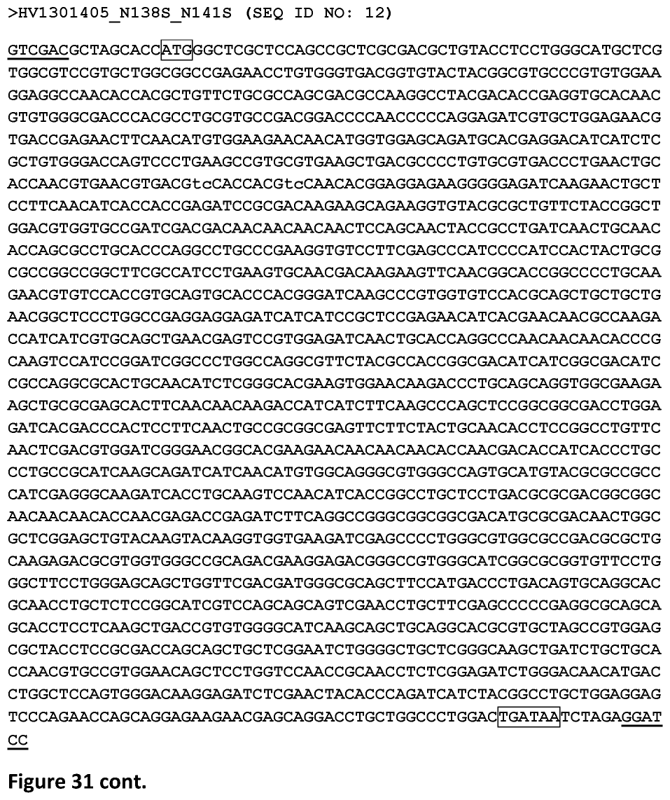

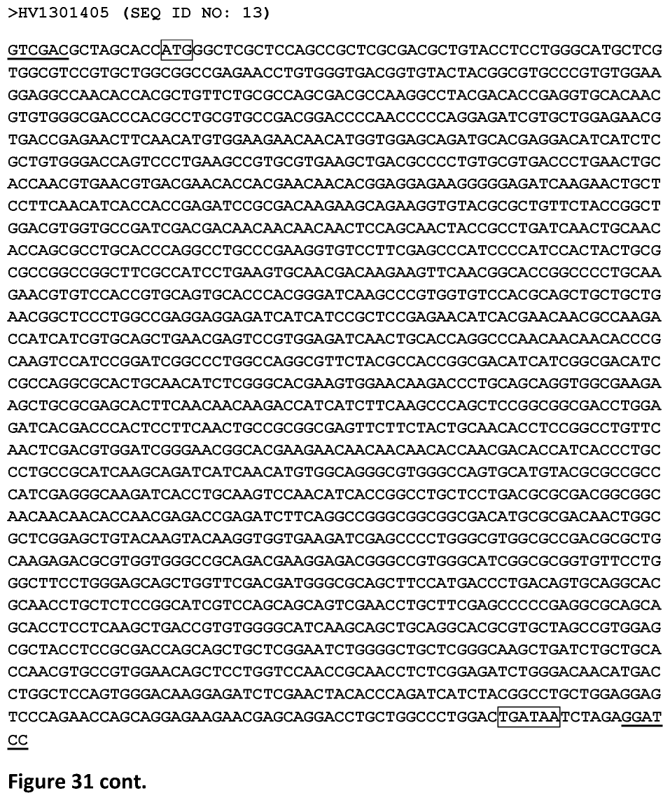

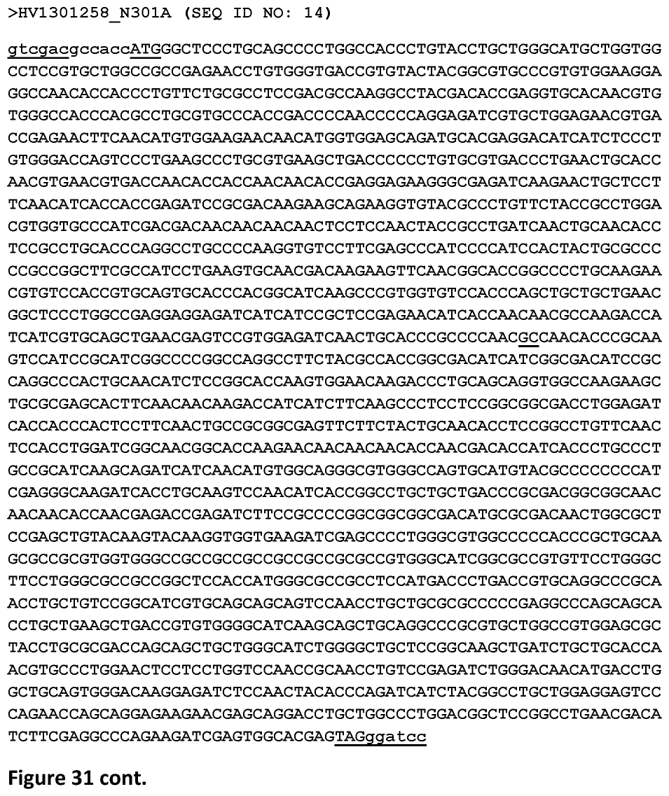

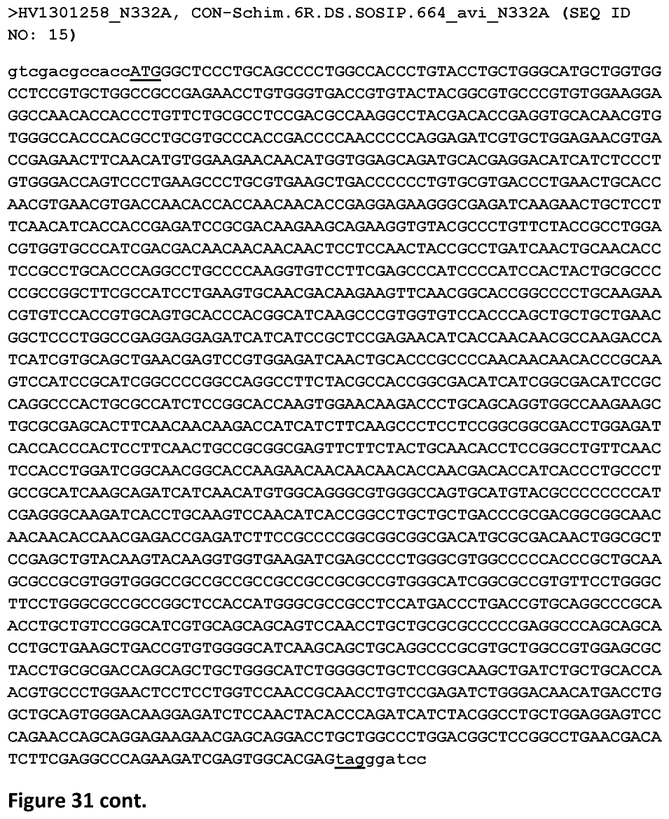

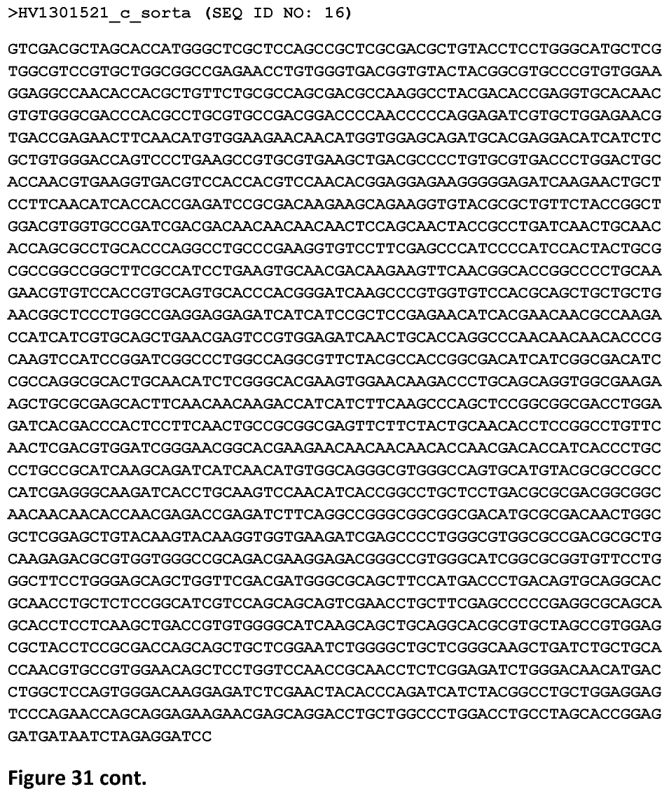

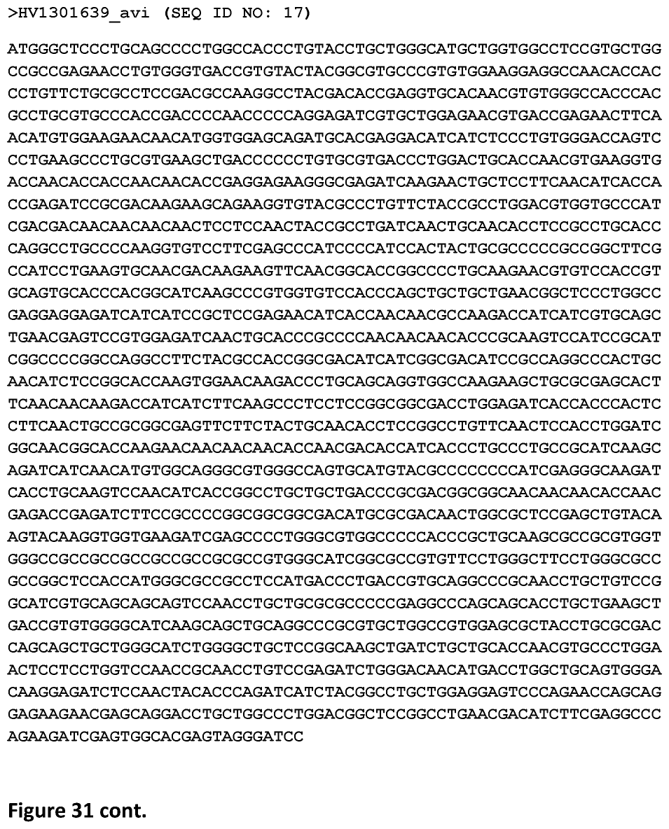

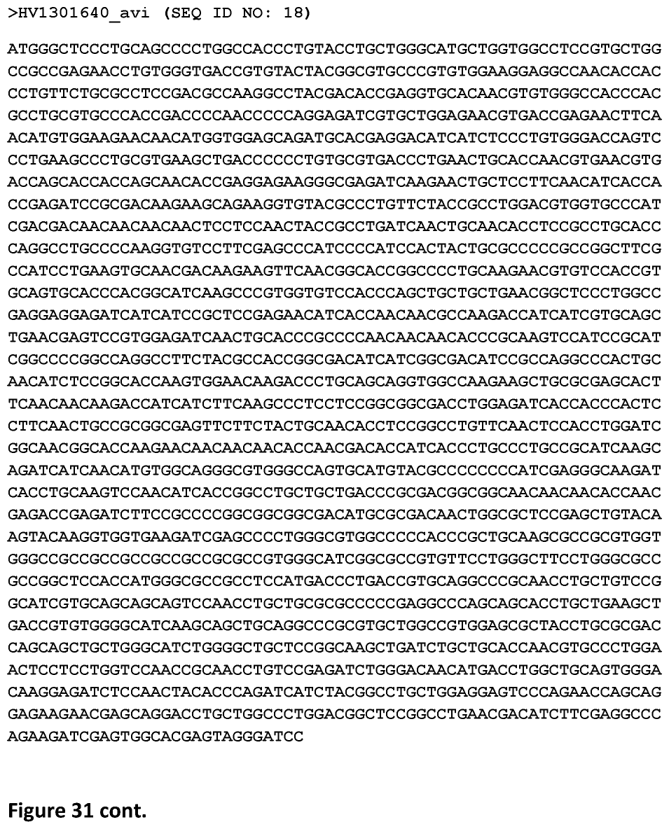

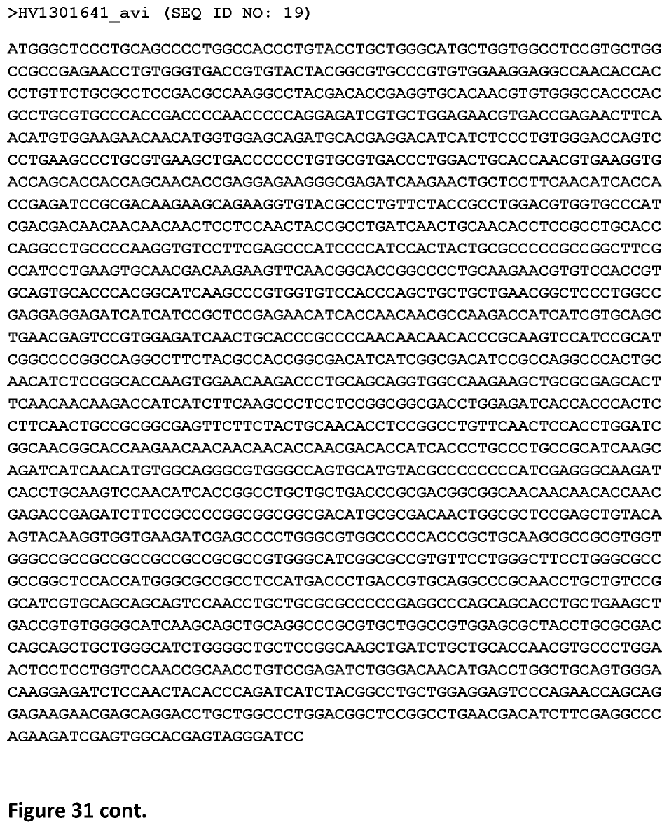

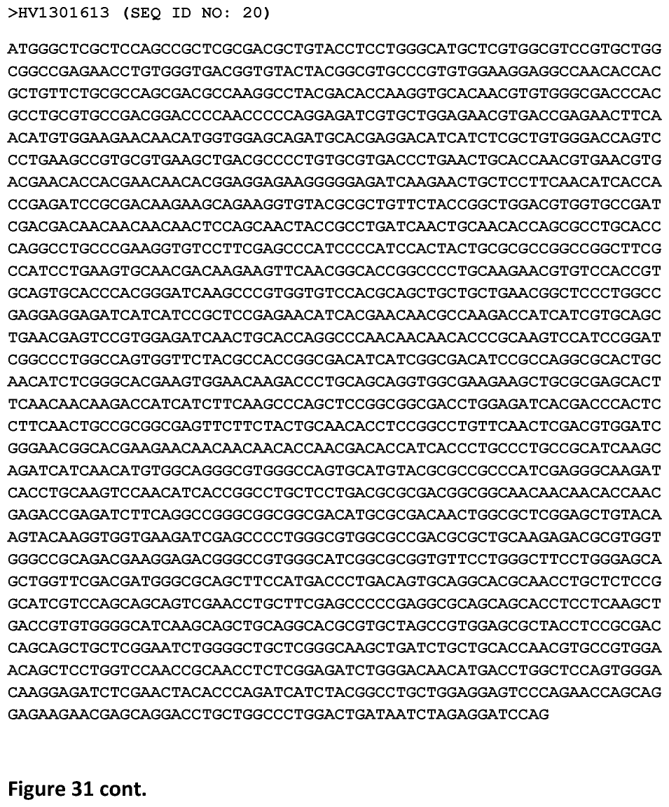

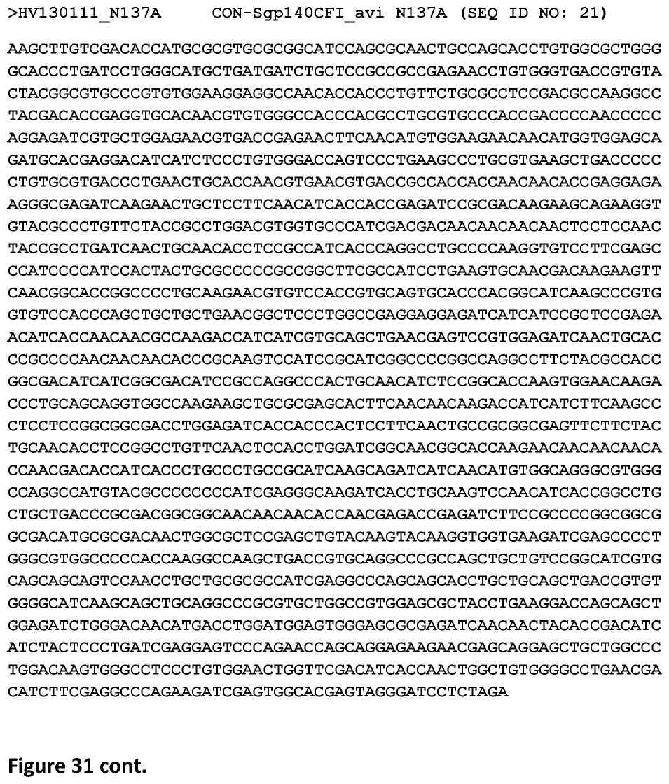

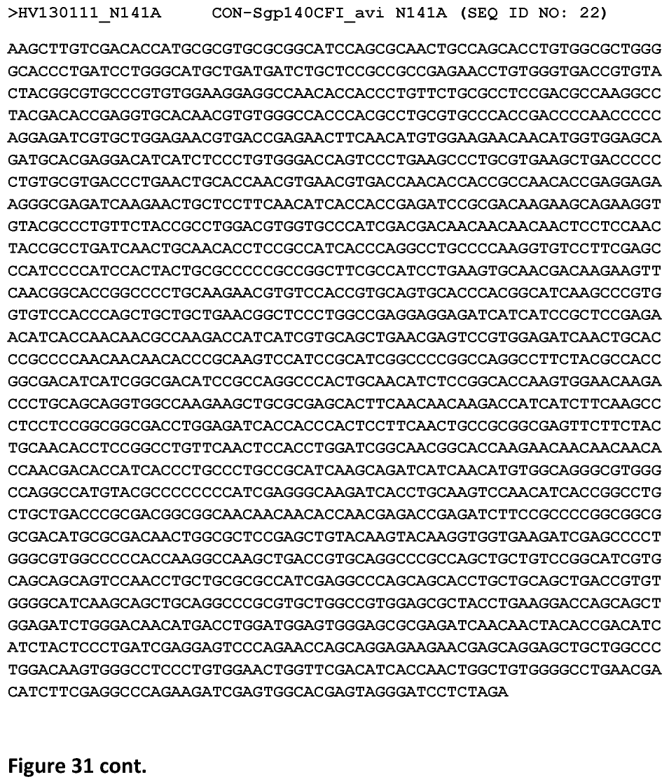

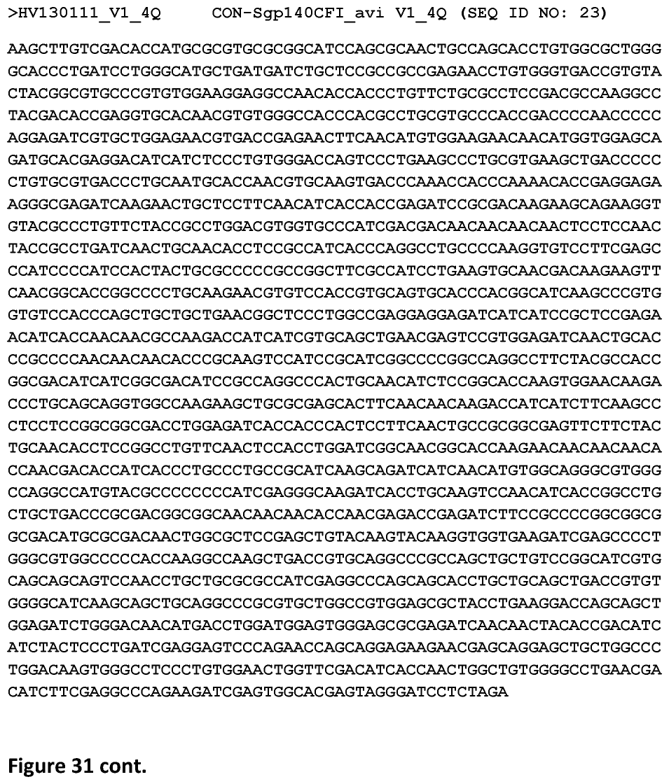

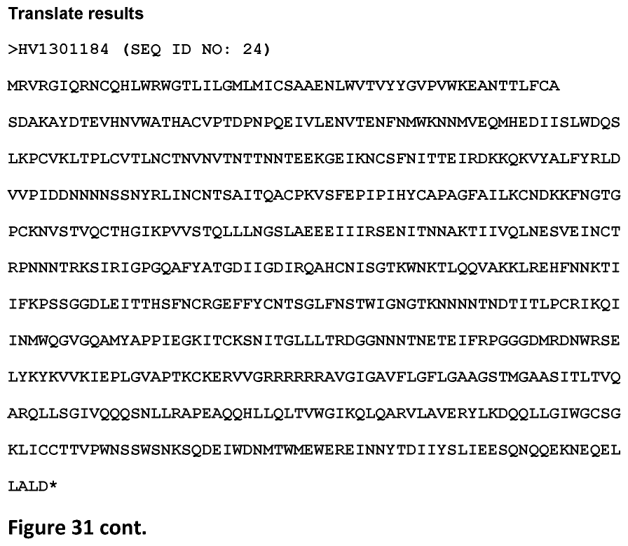

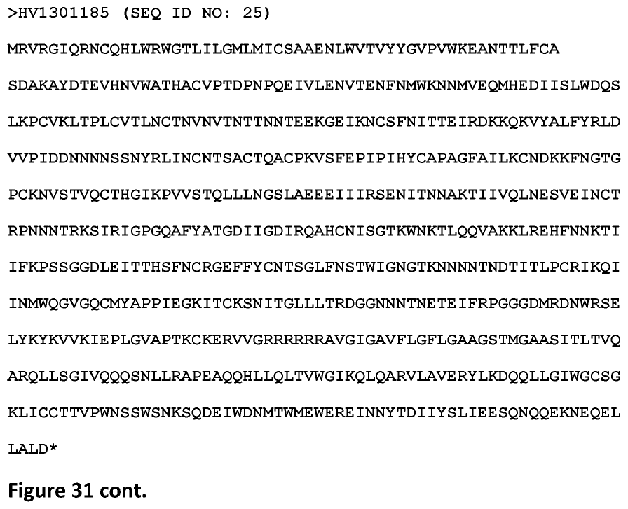

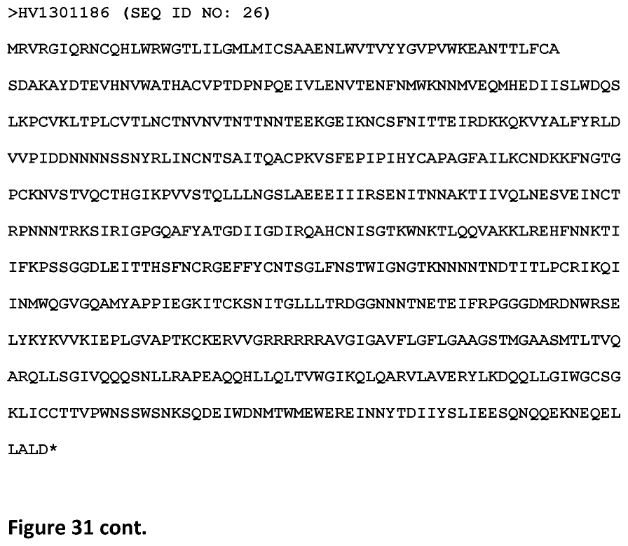

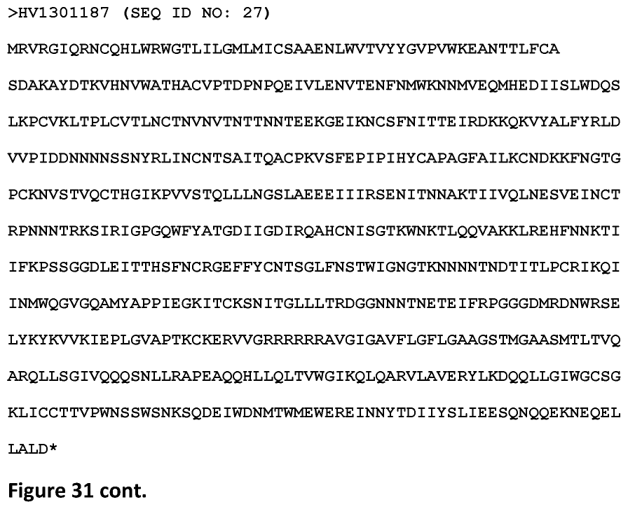

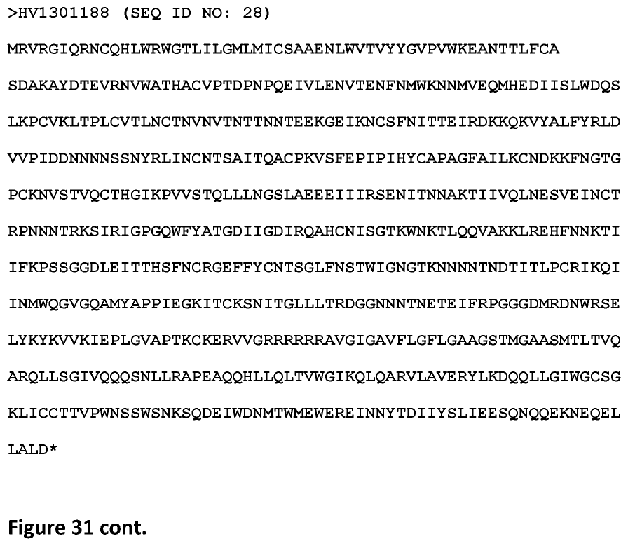

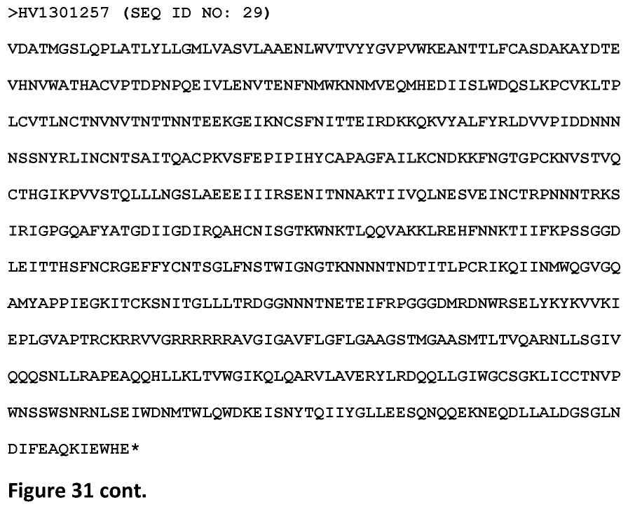

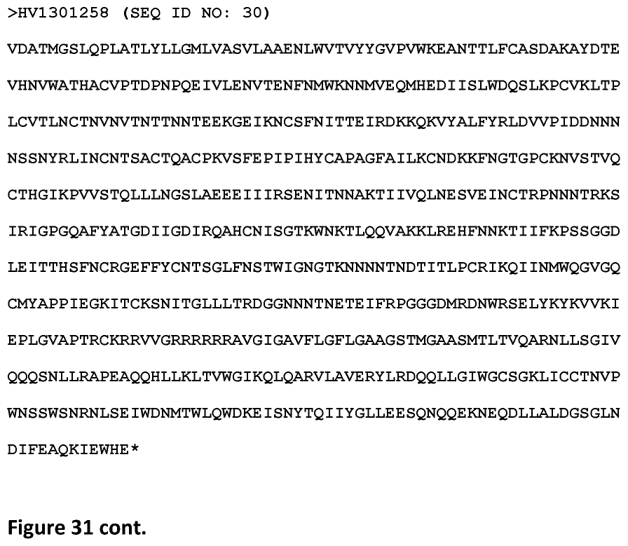

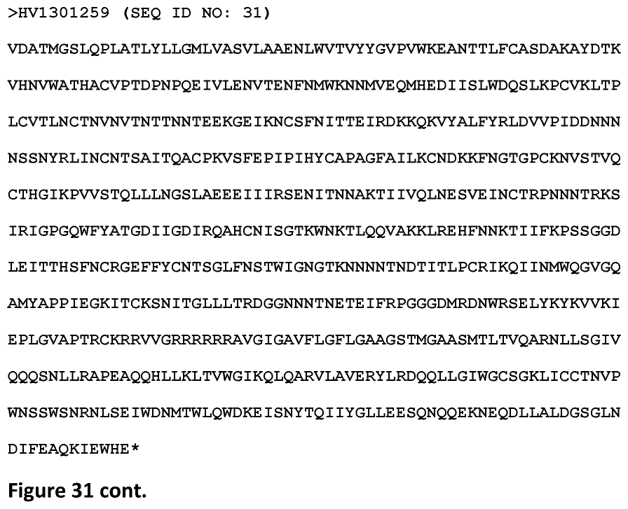

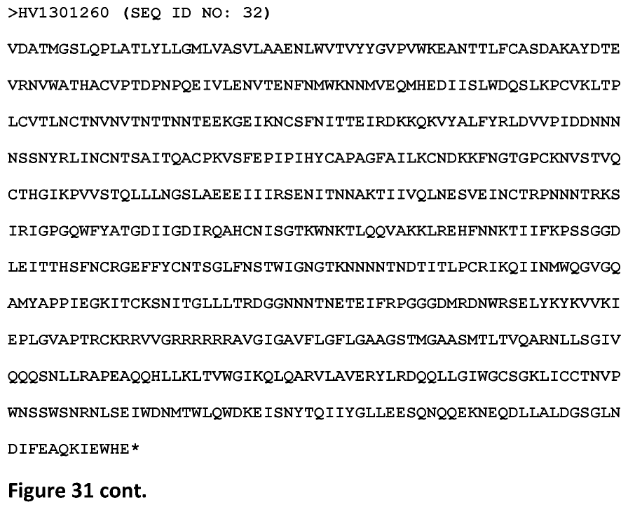

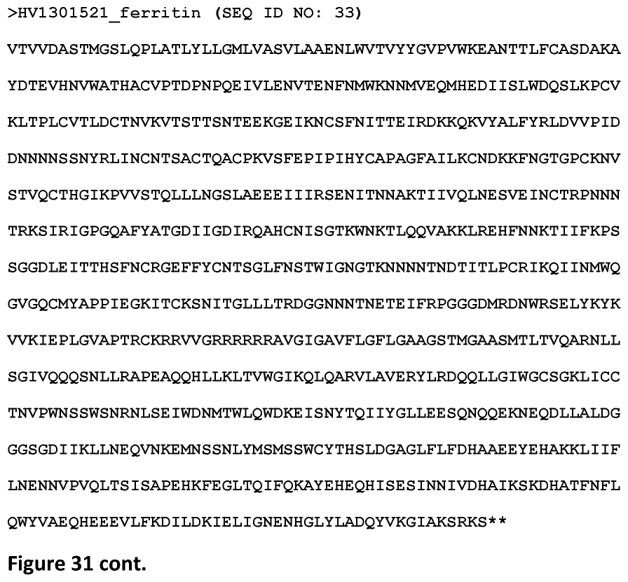

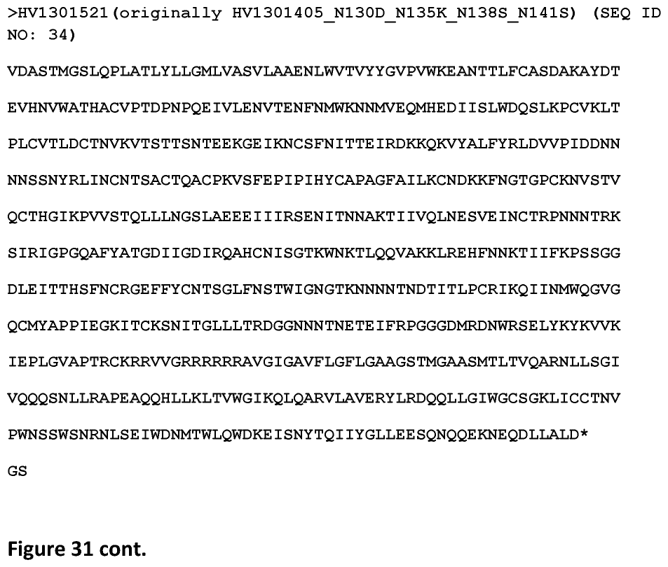

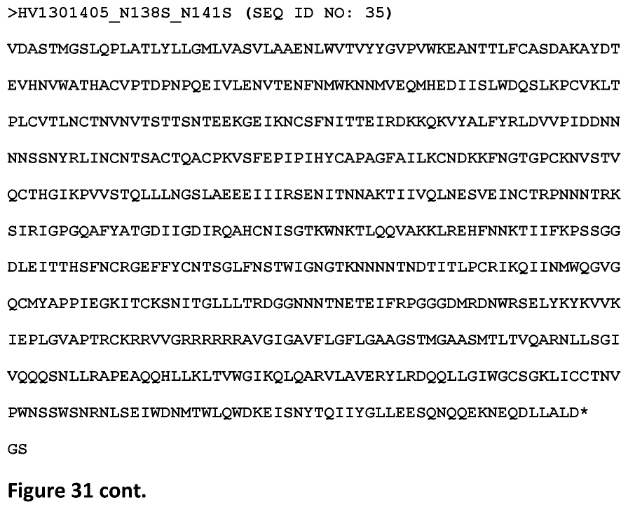

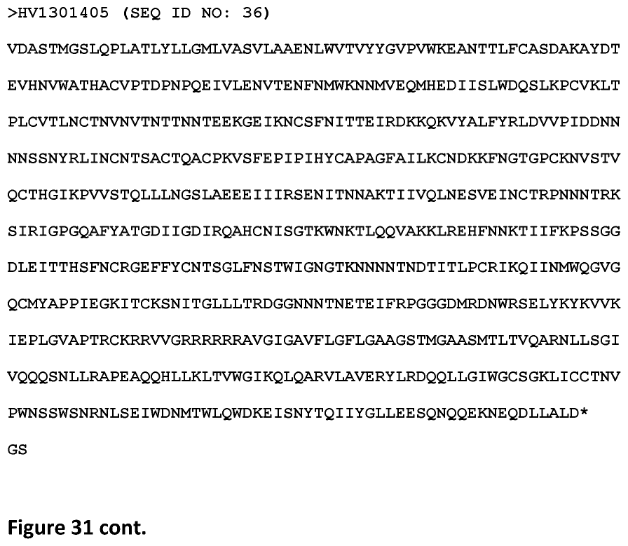

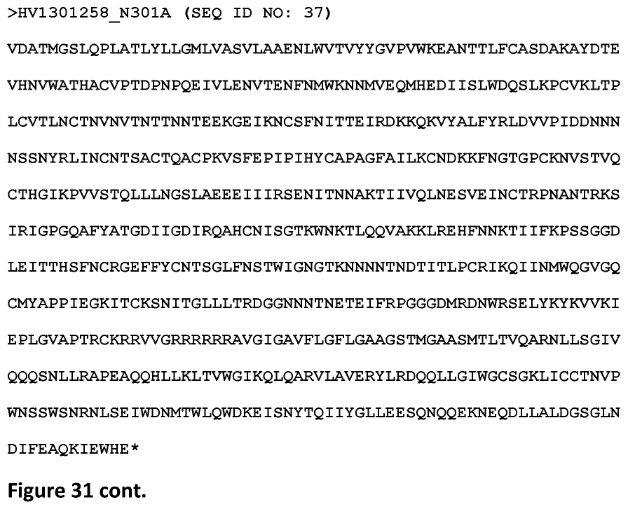

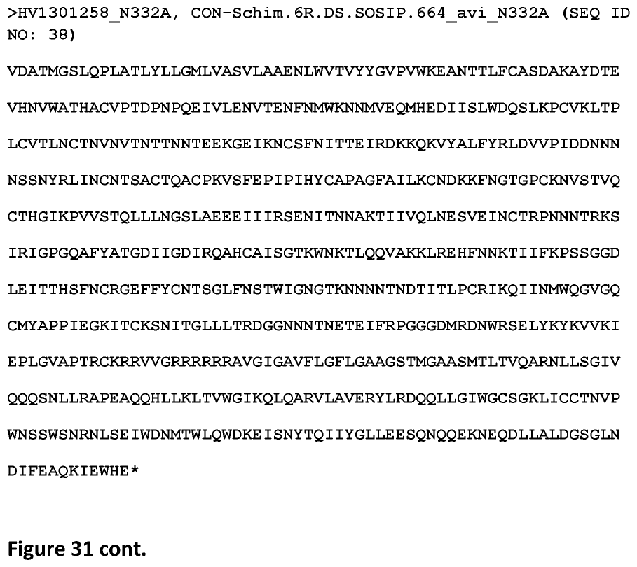

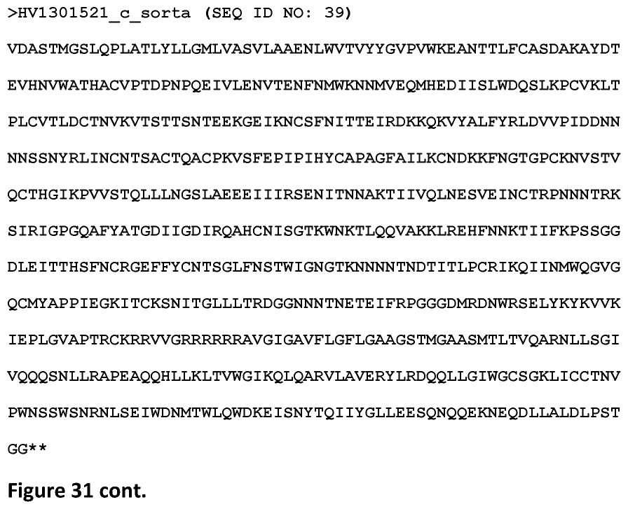

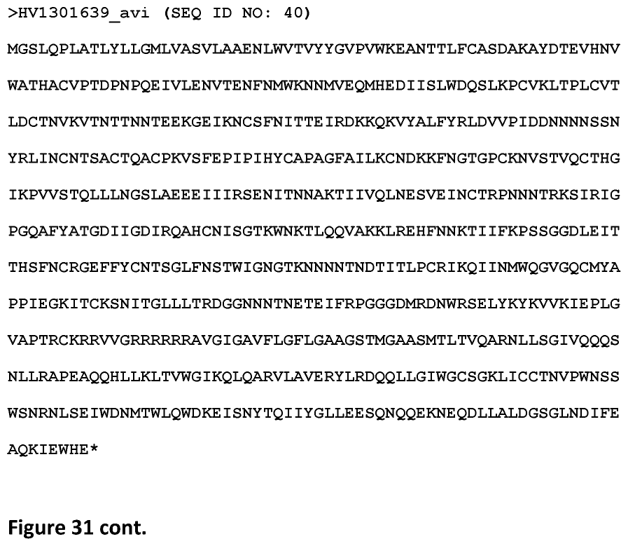

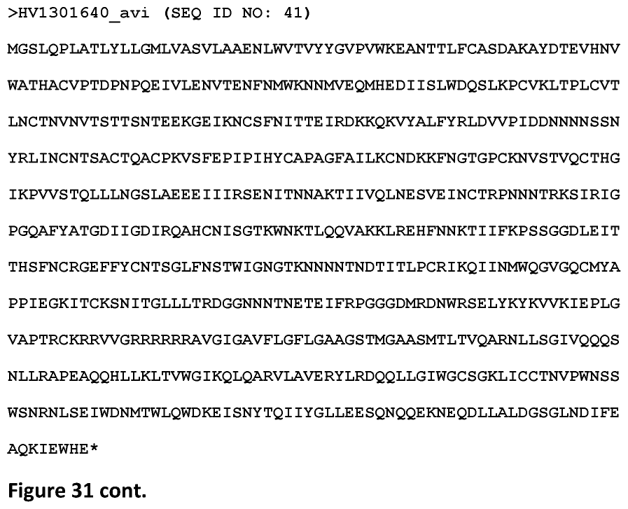

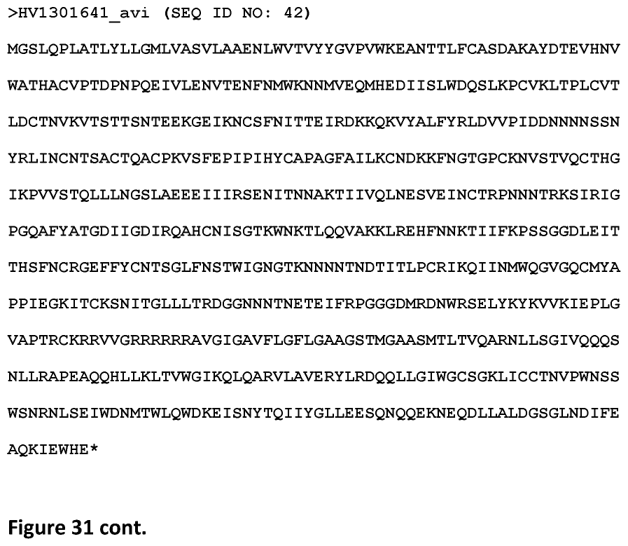

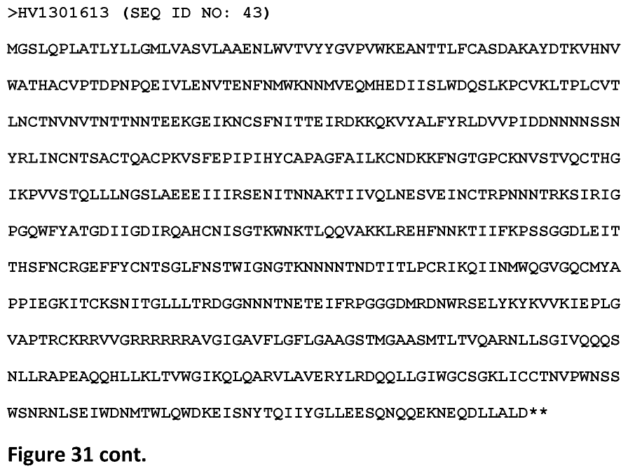

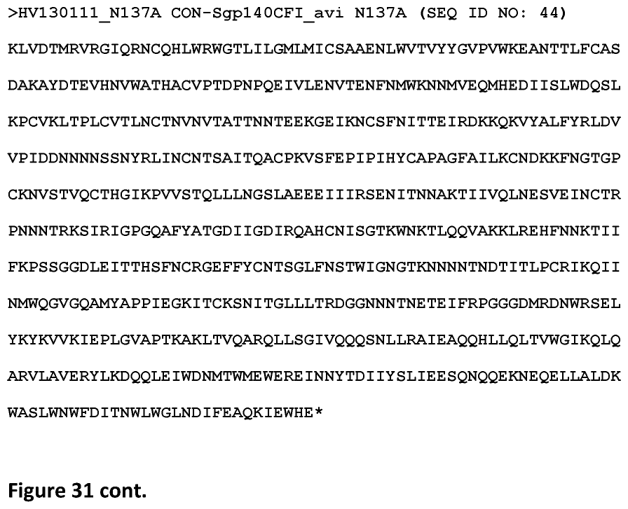

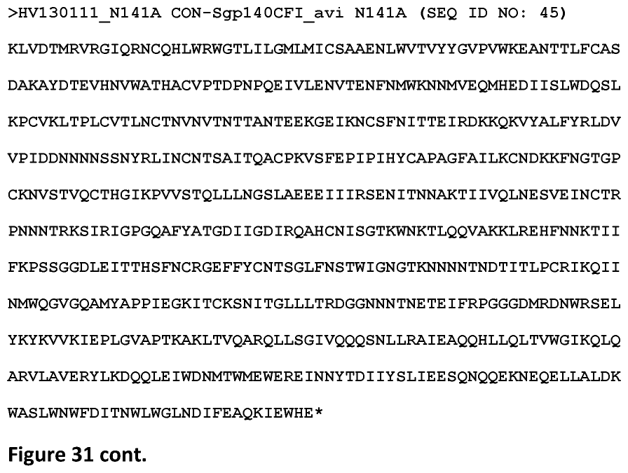

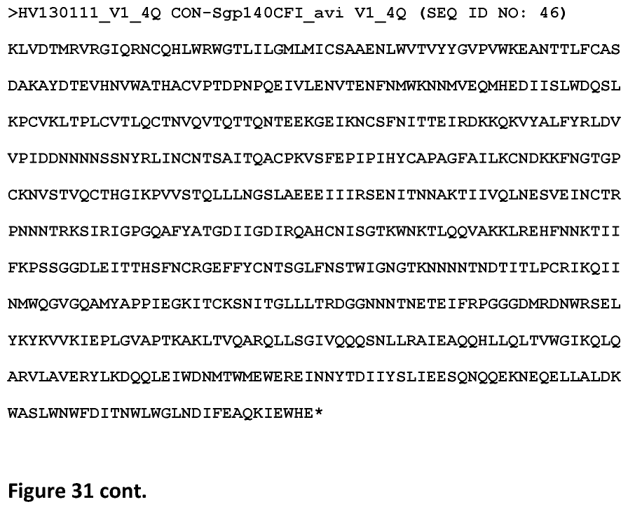

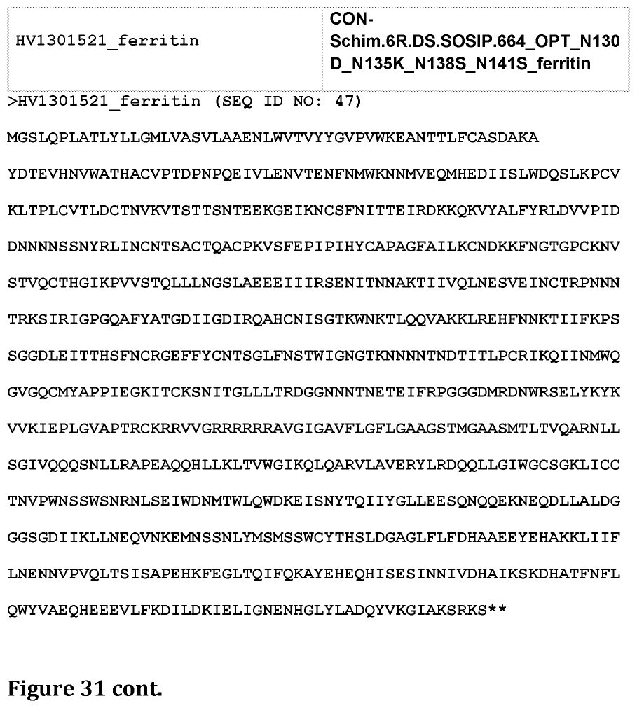

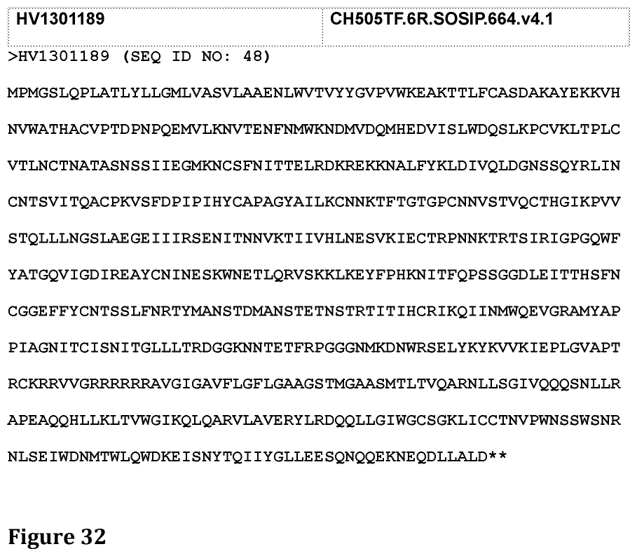

See also Example 4 for sequences and FIG. 31, and FIG. 32.

Columns and Buffers:

TFF filters used for buffer exchange and concentration of clarified harvest were single-use polyethersulfone (PES) SIUS PD 300 kD Cassettes from Repligen. Toyopearl NH2 750F chromatography was performed by packing loose Toyopearl NH2 750F resin from Tosoh Biosciences in a GE Healthcare 26/20 HiScale column housing. CHT Type 1 40 μm chromatography was performed using a Bio-Scale Mini CHT Type 1 cartridge from BioRad. Capto Phenyl chromatography was performed with the Capto Phenyl (high sub) prepacked HiScreen column from GE Healthcare. Capto DeVirS chromatography was performed using loose Capto DeVirS resin from GE Healthcare in a Millipore Sigma Vantage® L Laboratory Column VL 11×250. Phenyl Sepharose Chromatography was performed with loose Phenyl Sepharose 6 Fast Flow (high sub) resin in a Kinesis Omnifit EZ: Column 6.6 mm ID/250 mm (PN 006EZ-06-25-FF) housing to a bed height of 17 cm. All buffer components used in these purification methods were of a quality suitable for GMP production and were from SAFC (BisTris) and Avantor (all other chemicals). Water used for buffer preparation was ultrapure water produced with Millipore Direct 16 system.

Preparation of reference standards of SOSIP trimers and nanoparticles˜as referenced (13)

Stabilized CH505 TF ch.SOSIP trimers were produced by transient transfection of Freestyle293 cells using 293fectin (Invitrogen) as the transfection reagent (Vaccine Induction of Heterologous Tier 2 HIV-1 Neutralizing Antibodies in Animal Models, Saunders K. et al., Cell Rep., 2017 Dec. 26, 21(13):3681-3690, PMID: 29281818). Each liter of Freestyle293 cells received 650 μg of plasmid DNA encoding the SOSIP trimer and 150 μg of furin-encoding plasmid DNA. Six days post transfection the supernatant was cleared of cells, concentrated, and subjected to PGT145 affinity chromatography. PGT145 columns were made by conjugating 100 mg of PGT145 to 10 mL of sepharose fast flow resin (GE Healthcare). Trimeric envelope was purified in 10 mM Tris pH8, 500 mM NaCl on a HiLoad Superdex200 16/600 column (GE Healthcare). All proteins were snap-frozen and stored at −80° C.

ELISA—as referenced (8)

Sandwich ELISA for Trimer-2 and Nanoparticle-2 Titer and Antigenicity Assessments

Microtiter plates are coated with capture antibody, PGT128 Fab, and incubated overnight at 4° C. Plates are washed four times with PBS containing 0.05% Tween-20 (PBST) between each incubation step of the assay. Coated plates are blocked with a solution of PBS containing 1% BSA. Reference standard, samples and controls are prepared in assay diluent (PBST with 1% BSA), added to the plate and incubated for 1 hour at room temperature. The indicated detection antibody is added to the plate and incubated at room temperature for 1 hour. A goat-anti-human Fc polyclonal antibody that is conjugated to horseradish peroxidase (HRP) enzyme is then added to plate and incubated at room temperature for 1 hour. Substrate, tetramethylbenzidine (TMB), is then added to the plate. The reaction is quenched and the absorbance value is read on a microtiter plate reader. An evaluation of the concentration in the sample is determined by extrapolation from a standard curve of varying concentrations of reference standard, where the response of the standard concentrations has been fit to a 4-parameter logistic equation.

Biolayer Interferometry

The antigenicity characterization of trimeric SOSIP proteins and nanoparticles was performed by biolayer interferometry (BLI) using the ForteBio OctetRed96. Monoclonal antibodies that bind to several key epitopes of the HIV envelope protein were prepared at 20 ug/mL in PBS and captured using AHC (Anti-hIgG) biosensors for 300 s to a level of approximately 1.5-2.0 nm. The antibody captured sensor tips were washed with PBS for 60 s and then dipped into the SOSIP protein or NP diluted down to 25 μg/mL or 50 ug/mL in PBS for an association length of 400 s. After this association step, the sensors were placed back into PBS for a dissociation length of 600 s. The antigenicity analysis of SOSIP proteins and NPs was performed using the ForteBio Data Analysis 10.0 software. For data processing, the Y-axis was aligned to the baseline from 115 s to 119.8 s; the inter-step correction was aligned to the dissociation step to account for jumps in signal; and the flu specific antibody CH65 was used as a negative control for reference subtraction. Followed by reference subtraction, the response of the SOSIPs against each antibody was calculated at the end of the association period from 390.0 to 395.0 s. The calculated response was normalized relative to either PGT151 or PGT128 bnAb binding response and reported as % relative binding (15).

Negative stain—electron microscopy: SOSIP trimers were with diluted to 20-40 μg/ml with HEPES buffered saline (20 mM HEPES, 150 mM NaCl, 2% glycerol, pH 7.4). Copper EM grids (400 mesh) with carbon film were glow discharged at 15 mA for 20 seconds. Immediately after glow discharge, 5 μl of diluted trimer was placed onto the carbon film, incubated for 10 seconds, washed with 5 μl of deionized water for 5 seconds, and then stained with 5 μl of 0.5-0.6% uranyl formate in dionized water for 60 seconds. Uranyl formate solution was then blotted off and the grid allowed to air dry. All steps were done at room temperature (20-22° C.). Dried grids were imaged on a Philips EM420 at 100 kV accelerating voltage with a 2k×2k CCD camera at 49,000× magnification, corresponding to 6.9 Å/pixel. Images were analyzed with the EMAN2.2 image processing software suite. Approximately 20,000 individual trimers were automatically boxed out of the images using the Swarm particle picker in EMAN2, with box size of 48 pixels, and particle stacks were subjected to 4 iterations of 2D class averaging using the default settings in EMAN2, with 32 classes. Sample lots were qualitatively scored 1-5 based on the number of non-trimer classes (1=0-1 non-trimer classes, 2=2-4, 3=5-9, 4=10-15, 5=more than 16 non-trimer classes).

SDS-PAGE

Non-reduced and reduced SDS-PAGE analysis was performed using 4 to 12% BisTris NuPAGE SDS-PAGE with ThermoFisher gel systems. Reduced samples were incubated for 5 minutes at 95° C. in the presence of NuPAGE Sample Reducing Agent from ThermoFisher. Staining was performed with coomassie Simply Blue SafeStain from ThermoFisher. Precision Plus Protein Standard from BioRad was used as the molecular weight marker. For purified samples, loading was targeted at 3 μg/well and for crude samples loading was performed with an amount sample sufficient to visualize the global protein population present in the sample.

Results

Overview of Purification Methods for Soluble SOSIP Trimers

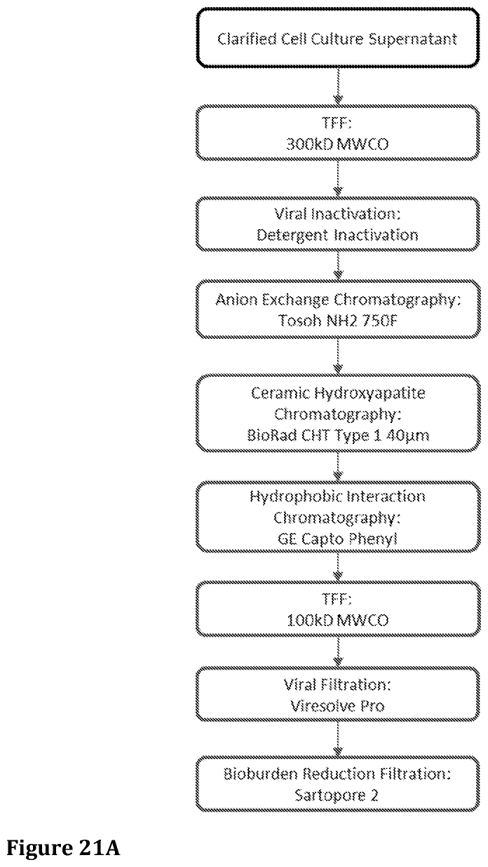

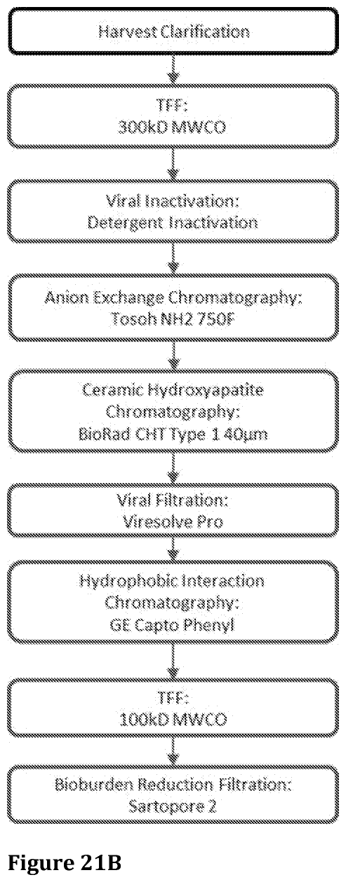

In general, the methods described include several types of column chromatography, and demonstrate that three variations of sequential columns can be used. The overall schema for three variations of the methods are shown in FIG. 1A. These three methods are designed to remove product-related impurities and result in purified Env trimers that are well-folded, and homogeneous in negative-stain electron microscopy (nsEM). When SOSIP HIV-1 Env trimers are produced under GMP conditions for clinical or commercial use, additional steps will be added to the purification process to include bioburden reduction filtration steps and viral clearance steps that are routine in the production of biological products from cell culture to fulfill regulatory criteria for cGMP protein production for human clinical trial use (16). Additional chromatography steps may also be added to give additional reduction of host cell contaminants (HCP and HC-DNA). An exemplary clinical cGMP process can be seen in FIG. 21A or 21B.

Method 1 for CH505 Transmitted/Founder (T/F) Stabilized SOSIP Trimer: Three-Step Purification Using Anion Exchange and Ceramic Hydroxyapatite Chromatography.

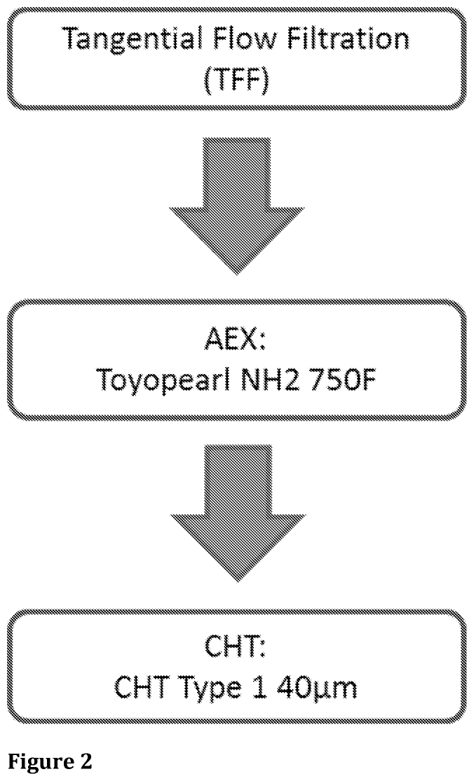

Using the CH505 transmitted/founder (TF stabilized SOSIP trimer (13)) recombinantly expressed in CHO DG44 cells, we developed an affinity-free purification process. In one version (Method 1), the isolation of trimer Env was accomplished in three unit operations (FIG. 2), starting with tangential flow filtration (TFF), followed by anion exchange (AEX) chromatography, and ceramic hydroxyapatite chromatography (CHT).

TFF, also known as ultrafiltration and diafiltration, is a method for separating molecules based on size. Membranes with specific pore sizes, typically indicated by most vendors with a molecular weight cutoff, are used under pressure generated by tangential flow of liquid. Molecules smaller than the molecular weight cutoff are sieved through the membrane leaving behind larger molecules. For HIV-1 Env, the differences in size between trimer and smaller contaminants, including product variants can be utilized as an effective purification step.

The purification schema for soluble CH505 TF stabilized SOSIP trimers in Method 1, depicted in FIG. 2, used TFF to selectively retain HIV-1 Env and prepare clarified harvest for the initial chromatography step. Clarified harvest was ultrafiltered and diafiltered through a membrane with specific pore size capable of separating trimer Env and smaller product variants. The membrane was constructed from modified polyethersulfone (mPES) with 300 kD molecular weight cutoff. The first chromatography step in Method 1 used Toyopearl NH2 750F to bind and elute the target molecule into a single, bulk fraction. Toyopearl NH2 750F is a salt tolerant anion exchanger with a wide pH range for loading. For HIV-1 Env, it is recommended to bind at a unique pH for a typical anion exchanger. HIV-1 Env was bound to the resin at pH 7.2 and at an ionic strength that allows binding of HIV-1 Env (250 mM NaCl). While bound to the column, HIV-1 Env was washed with equilibration buffer. HIV-1 Env enriched trimeric gp140 is selectively eluted by increasing the ionic strength. Table 1 depicts the Toyopearl NH2 750F step-by-step operation including post-product cleaning steps.

TABLE 1
Anion Exchange Chromatography by Toyopearl NH2 750F
step by step process

| | | Column | Flow |
|---|---|---|---|
| | | Volume | Rate |
| Step Name | Buffer | (CV) | (cm/hr) |
| Equilibration | 20 mM HEPES, 250 mM | 5 | 200 |
| | NaCl, pH 7.2 | | |
| Load | pH 7.2, ~250 mM NaCl | ~30 L | 100 |
| | | Harvest/L | |
| | | Resin | |
| Wash 1 | 20 mM HEPES, 250 mM | 5 | 100 |
| | NaCl, pH 7.2 | | |
| Eluate | 20 mM NaHEPES, 0.6 M | 6 | 200 |
| Collection | NaCl, pH 7.2 | | |
| Strip | 50 mM Acetate, 3 M NaCl | 4 | 200 |
| Sanitization | 0.5 M NaOH | 5 | 100 |
| Rinse | 20 mM HEPES, 250 mM | 5 | 200 |
| | NaCl, pH 7.2 | | |
| Storage | 20% Ethanol | 5 | 200 |

The second chromatography step in Method 1 used Ceramic Hydroxyapatite (CHT) Type 1 resin to bind HIV-1 Env trimers in the NH2 eluate buffer matrix. While bound to the column, HIV-1 Env trimers were washed with NH2 Elution buffer. Trimeric gp140 Env-1 was then selectively eluted by increasing the phosphate concentration (Elution 1). Table 2 depicts the executed CHT step-by-step operation including pre-product and post-product cleaning steps.

TABLE 2
Ceramic hydroxyapatite (CHT) chromatography by CHT
Type 1 40 μm step by step instructions

| | | Column | Flow |
|---|---|---|---|
| | | Volume | Rate |
| Step Name | Buffer | (CV) | (cm/hr) |
| Rinse | 20 mM HEPES, 30 mM | 1 | 200 |
| | NaPhosphate, pH 7.2 | | |
| Charge | 400 mM NaPhosphate | 3 | 200 |
| Equilibration | 20 mM HEPES, 600 mM | 5 | 200 |
| | NaCl, pH 7.2 | | |
| Load | NH2 Eluate | NA | 200 |
| Wash 1 | 20 mM HEPES, 600 mM | 5 | 200 |
| | NaCl, pH 7.2 | | |
| Wash 2 | 20 mM HEPES, pH 7.2 | 5 | 200 |
| Elution 1 (25Au- | 20 mM HEPES, 30 mM | 5 | 200 |
| 25Au) | NaPhosphate, pH 7.2 | | |
| Elution 2 (25Au- | 20 mM HEPES, 100 mM | 5 | 200 |
| 25Au) optional | NaPhosphate, pH 7.2 | | |
| Strip (25Au- | 400 mM NaPhosphate | 5 | 200 |
| 25Au) | | | |
| Rinse | 20 mM HEPES, 30 mM | 1 | 200 |
| | NaPhosphate, pH 7.2 | | |
| Sanitization | 0.5 M NaOH | 5 | 100 |

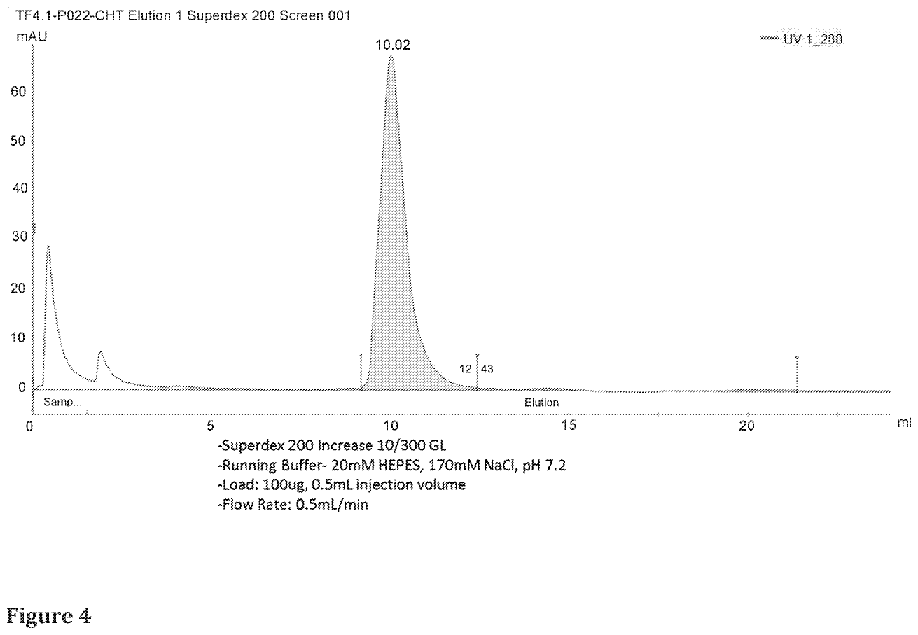

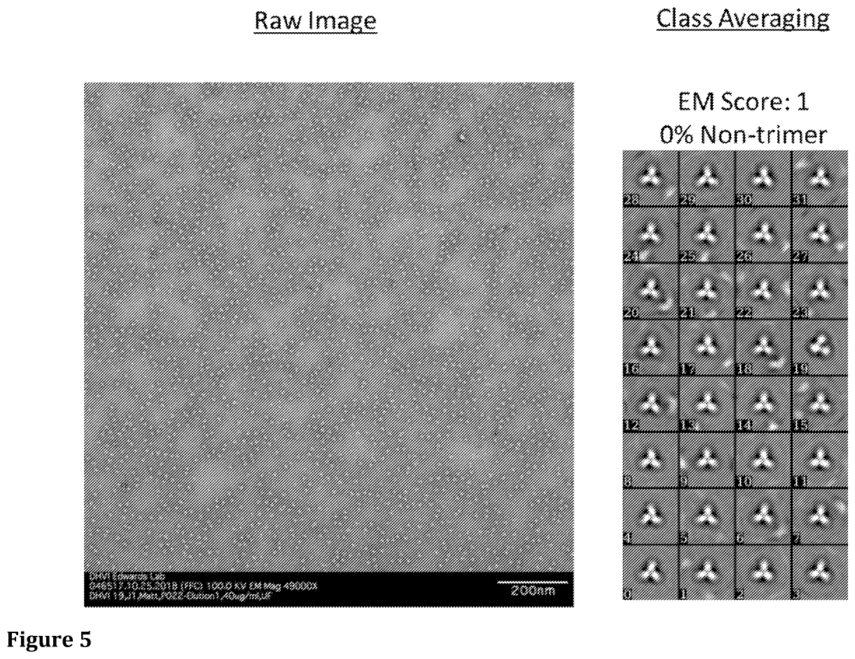

FIG. 3 shows the purity after ceramic hydroxyapatite chromatography by non-reduced SDS-PAGE, FIG. 4 shows the purity of the Env trimer by SEC, and FIG. 5 shows trimer homogeneity by negative stain electron microscopy (nsEM).

In certain embodiments, CHT step includes only one elution step—Elution 1.

Method 2 for CH505 Transmitted/Founder Stabilized SOSIP Trimer: Three-Step Purification Using Anion Exchange and Hydrophobic Interaction Chromatography.

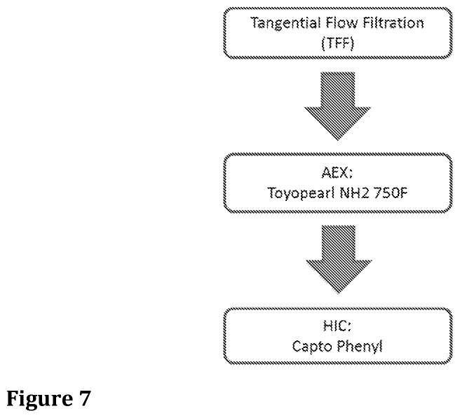

The initial purification operation in Method 2 used TFF to selectively retain HIV-1 Env and prepare clarified harvest for the initial chromatography step. Clarified harvest was ultrafiltered and diafiltered through a membrane with specific pore size capable of separating trimer Env and smaller product variants. The membrane was constructed from modified polyethersulfone (mPES) with 300 kD molecular weight cutoff. The first chromatography step used in Method 2, FIG. 7, used Toyopearl 750F to bind and elute the target molecule into a single bulk fraction. Toyopearl NH2 750F is a salt tolerant anion exchanger with a wide pH range for loading. For HIV-1 Env, it is recommended to bind at a unique pH for a typical anion exchanger. HIV-1 Env was bound to the resin at pH 7.2 and at an ionic strength that allows binding of HIV-1 Env (250 mM NaCl). While bound to the column, HIV-1 Env was washed with equilibration buffer. HIV-1 Env enriched trimeric gp140 is selectively eluted by increasing the ionic strength. Table 1 depicts the Toyopearl NH2 750F step-by-step operation including post-product cleaning steps.

TABLE 3
Tosoh NH2 750F anion exchange chromatography
step by step process

| | | Column | Flow |
|---|---|---|---|
| | | Volume | Rate |
| Step Name | Buffer | (CV) | (cm/hr) |
| Equilibration | 20 mM HEPES, 250 mM | 5 | 200 |
| | NaCl, pH 7.2 | | |
| Load | pH 7.2, ~250 mM NaCl | ~30 L | 100 |
| | | Harvest/L | |
| | | Resin | |
| Wash 1 | 20 mM HEPES, 250 mM | 5 | 100 |
| | NaCl, pH 7.2 | | |
| Eluate | 20 mM NaHEPES, 0.6 M | 6 | 200 |
| Collection | NaCl, pH 7.2 | | |
| Strip | 50 mM Acetate, 3 MNaCl | 4 | 200 |
| Sanitization | 0.5 M NaOH | 5 | 100 |
| Rinse | 20 mM HEPES, 250 mM | 5 | 200 |
| | NaCl, pH 7.2 | | |
| Storage | 20% Ethanol | 5 | 200 |

The second chromatography step in Method 2 used Capto Phenyl resin to isolate trimeric gp140 HIV-1 Env in the unbound flow through fraction. NH2 Eluate was spiked to 0.6M ammonium sulfate and loaded onto the column, trimeric gp140 HIV-1 Env flowed through the column and was washed out with equilibration buffer. gp140 Env-1 impurities bound to the resin and were removed. Table 4 depicts the executed Capto Phenyl step-by-step operation including pre-product and post-product steps.

TABLE 4
GE Capto Phenyl step by step instructions

| | | Column | Flow |
|---|---|---|---|
| | | Volume | Rate |
| Step Name | Buffer | (CV) | (cm/hr) |
| Rinse | diH₂O | 2 | 300 |
| Equilibration | 20 mM HEPES, | 5 | 300 |
| | 0.6 M | | |
| | Ammonium Sulfate | | |
| | pH 7.2 | | |
| Load | NH2 Eluate spiked | NA | 200 |
| | to 0.6 M | | |
| | Ammonium Sulfate | | |
| Wash | 20 mM HEPES, | 5 | 200 |
| | 0.6 M | | |
| | Ammonium Sulfate | | |
| | pH 7.2 | | |
| Strip | diH₂O | 5 | 300 |
| Sanitization | 0.5 M NaOH | 5 | 100 |
| Rinse | diH₂O | 5 | 300 |
| Storage | 20% Ethanol | 5 | 300 |

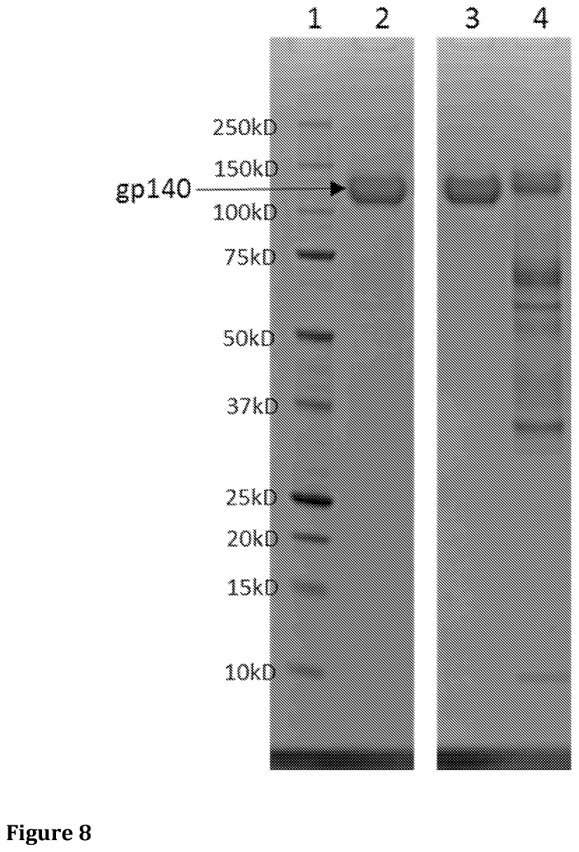

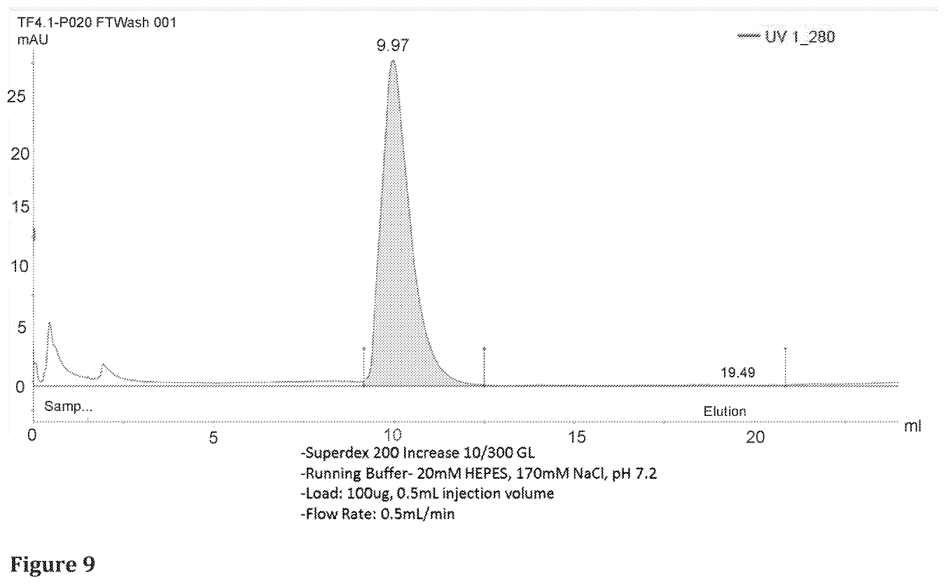

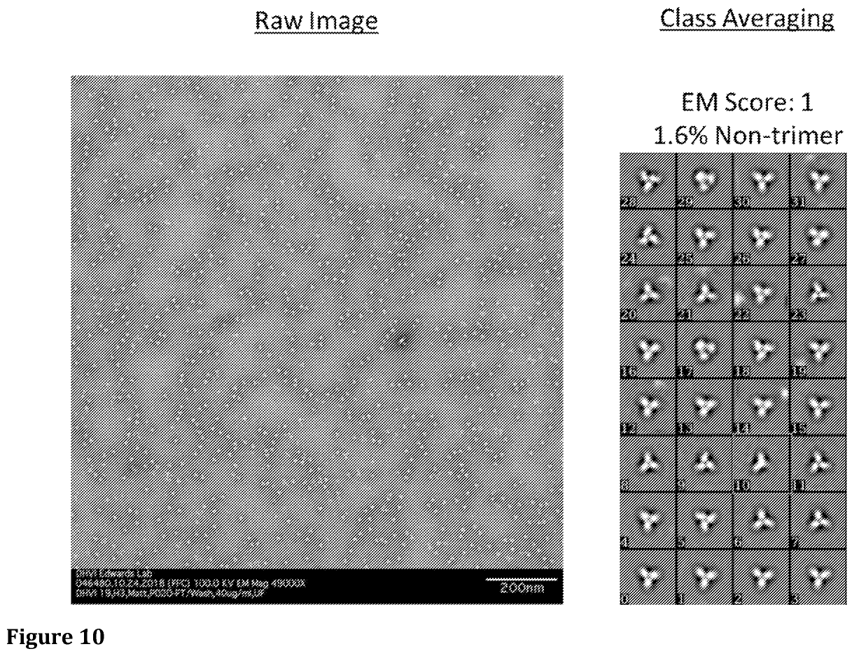

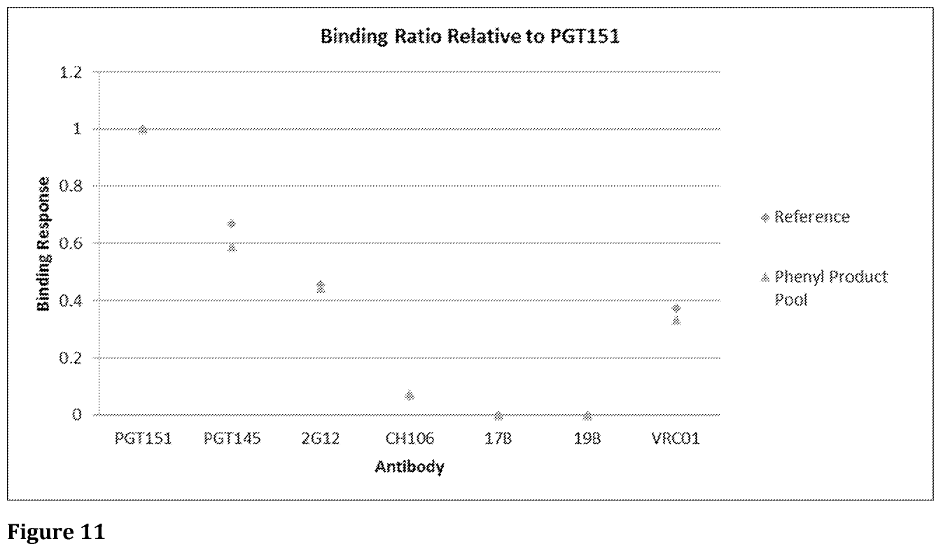

FIG. 8 shows the purity of the Capto Phenyl product pool after hydrophobic interaction chromatography by non-reduced SDS-PAGWE, FIG. 9 shows the purity of the Capto Phenyl product pool after HIC by SEC, and FIG. 10 shows the homogeneity of the CH505 TF stabilized SOSIP trimers by nsEM. FIG. 11 shows that trimers purified by Method 2 bind only to bnAbs and not to non-neutralizing antibodies.

Method 3 for CH505 Transmitted/Founder Stabilized SOSIP Trimer: Three-Step Purification Using Mix-Mode Cation Exchange/Heparin Like Affinity and Hydrophobic Interaction Chromatography.

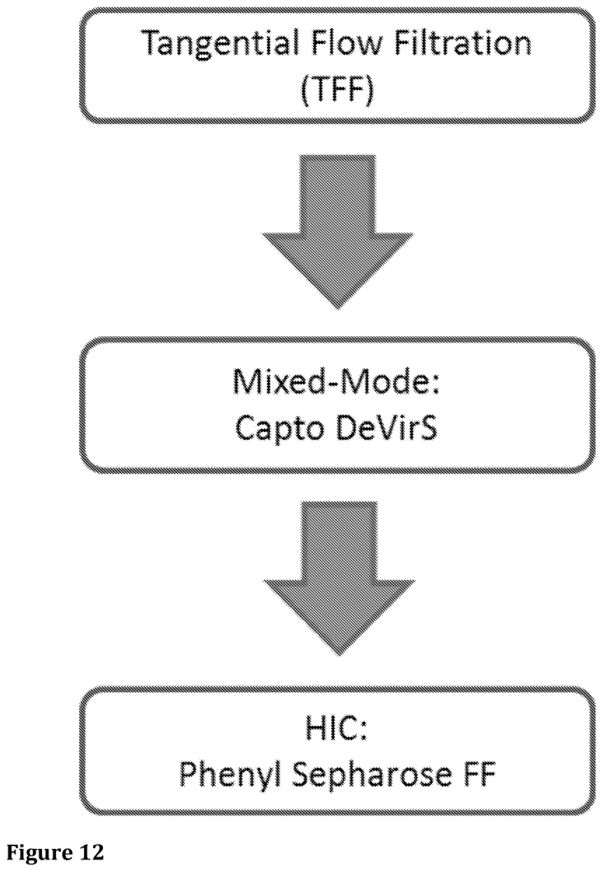

The purification process in Method 3 used TFF to selectively retain HIV-1 Env and prepare clarified harvest for the initial chromatography step (FIG. 12). Clarified harvest was ultrafiltered and diafiltered through a membrane with specific pore size capable of separating trimer Env and smaller product variants. The membrane was constructed from modified polyethersulfone (mPES) with 300 kD molecular weight cutoff.

The second purification step was executed using Capto™ DeVirS (GE Healthcare) chromatography. It has been demonstrated that the gp120 subunit has multiple heparin binding sites (17) and that heparin and its derivatives bind recombinant gp120 (18). Capto DeVirS combines cation exchange chromatography and a heparin analog (dextran sulfate) into a single mixed-mode media. The unique affinity-like properties of the ligand on the high capacity Capto bead effectively purifies HIV Env from host cell impurities. Moreover, the Capto™ agarose bead is base tolerant for cleaning and reuse. Concentrated and buffer exchanged clarified harvest was loaded onto the Capto DeVirS resin at pH 7.2 and low ionic strength. While bound to the column, HIV-1 Env was washed with equilibration buffer. Additional wash at an elevated pH (8.0) was then performed to further remove contaminants. HIV-1 Env was then eluted with increasing salt. Table 5 depicts the executed Capto DeVirS step-by-step operation including post-product cleaning steps.

TABLE 5
GE Capto DeVirS step by step process

| | | Column | Flow |
|---|---|---|---|
| | | Volume | Rate |
| Step Name | Buffer | (CV) | (cm/hr) |
| Equilibration | 15 mM BisTris, | 5 | 200 |
| | 15 mM HEPES, 12 | | |
| | mM NaCl | | |
| | pH 7.2 | | |
| Load | pH 7.2, ~1.5 mS/cm | TFF | 200 |
| | | Retentate | |
| Wash 1 | 15 mM BisTris, | 5 | 200 |
| | 15 mM HEPES, 12 | | |
| | mM NaCl | | |
| | pH 7.2 | | |
| Wash 2 | 25 mM HEPES, | 7 | 200 |
| | pH 8.0 (free acid) | | |
| Eluate | 50 mM NaHEPES, | 5 | 200 |
| Collection | 300 mM NaCl | | |
| (20mAu-20mAu) | pH 7.2 | | |
| Strip | 50 mM Tris, | 4 | 200 |
| | 2 M NaCl | | |
| Sanitization | 0.5 M NaOH | 5 | 100 |
| Rinse | 50 mM NaHEPES, | 5 | 200 |
| | 300 mM NaCl | | |
| | pH 7.2 | | |
| Storage | 20% Ethanol | 5 | 200 |

The third purification step in Method 3 used GE Healthcare's Phenyl Sepharose 6 FF (HIC) resin to bind and elute the target molecule into a single bulk fraction. DeVirS Eluate was diluted with 2.4M ammonium sulfate to a final ammonium sulfate of 1.8M and loaded onto the HIC resin at pH 7.4. While bound to the column, HIV-1 Env was washed with equilibration buffer. HIV-1 Env was eluted with decreasing salt. Table 6 depicts the executed HIC step-by-step operation including pre-product and post-product cleaning steps.

TABLE 6
GE Phenyl Sepharose FF step by step instructions

| | | Column | Flow |
|---|---|---|---|
| | | Volume | Rate |
| Step Name | Buffer | (CV) | (cm/hr) |
| Water Rinse | Water | 5 | 150 |
| Pre-Sanitization | 0.5 M NaOH | 3 | 150 |
| 60 min. HOLD | N/A | N/A | N/A |
| Water | Water | 5 | 150 |
| Equilibration | 20 mM Tris, 1.8 M | 5 | 150 |
| | AmSO₄, pH 7.4 | | |
| Load | DeVirS Eluate | N/A | 150 |
| Wash | 20 mM Tris, 1.8 M | 10 | 150 |
| | AmSO₄, pH 7.4 | | |
| Gradient Elution | A: 20 mM Tris, 1.8 M | 20 | 150 |
| | AmSO₄, pH 7.4 | | |
| (A:B, 0-100%) | B: 20 mM Tris, pH 7.4 | | |
| Strip 1 | 20 mM Tris, pH 7.4 | 5 | 150 |
| Strip 2 | Water | 5 | 150 |
| Post Sanitization | 0.5 M NaOH | 5 | 150 |
| 60 min. HOLD | N/A | N/A | N/A |
| Water Rinse | Water | 5 | 150 |
| Storage | 20% Ethanol | 3 | 150 |

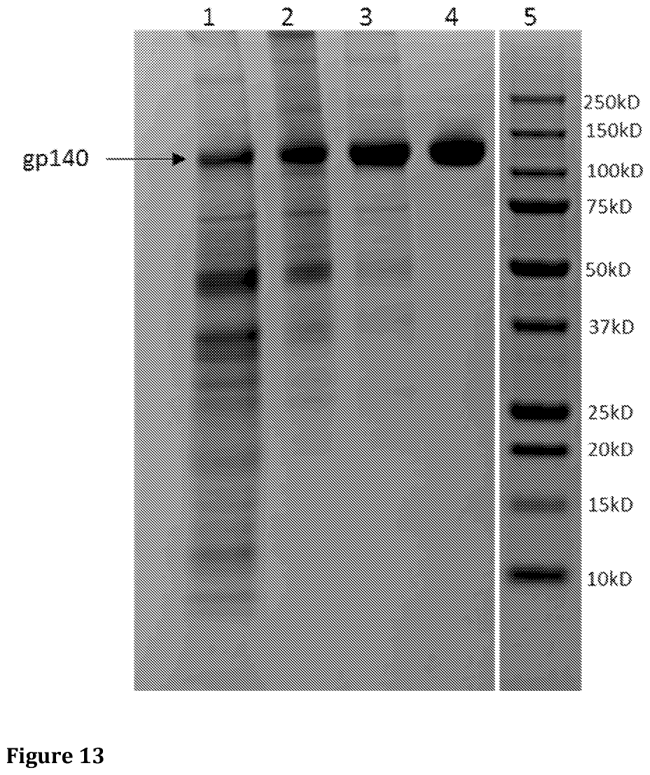

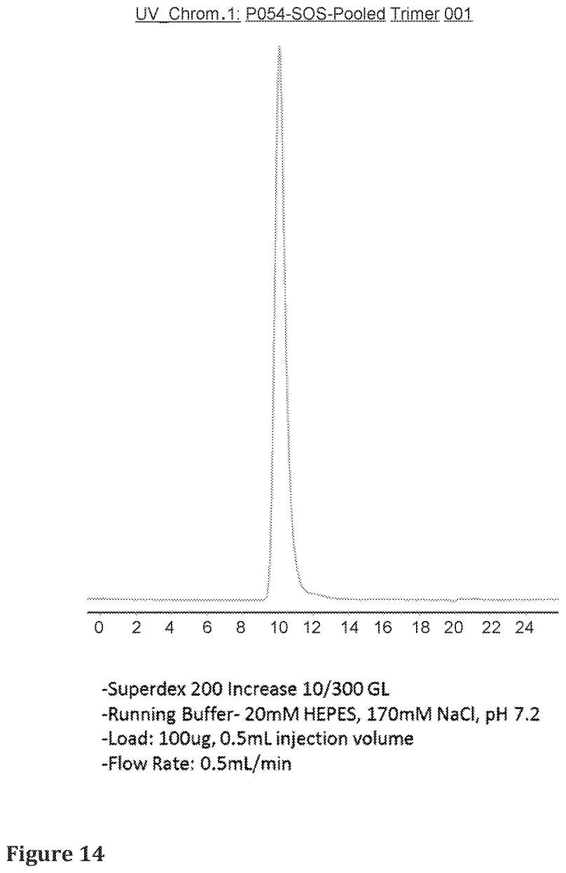

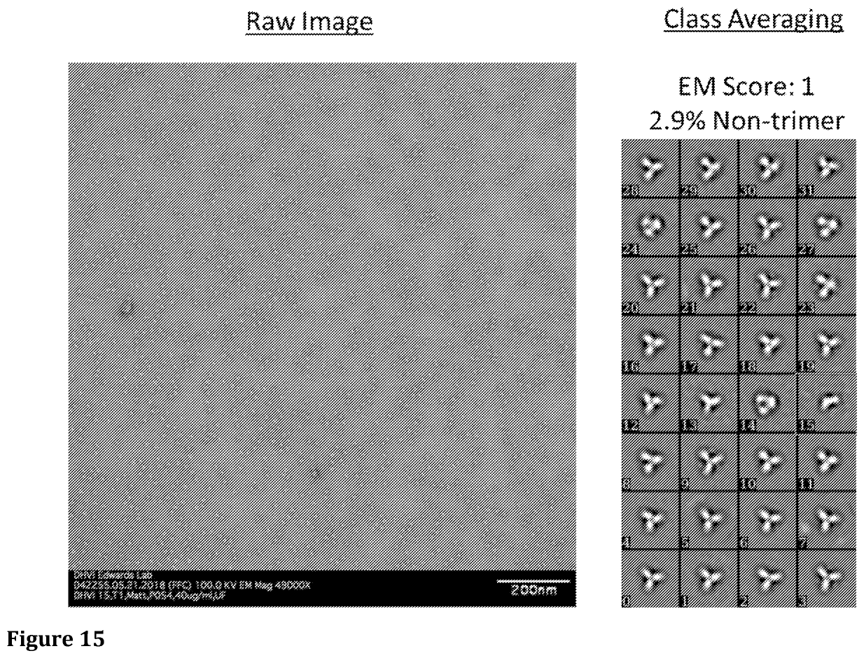

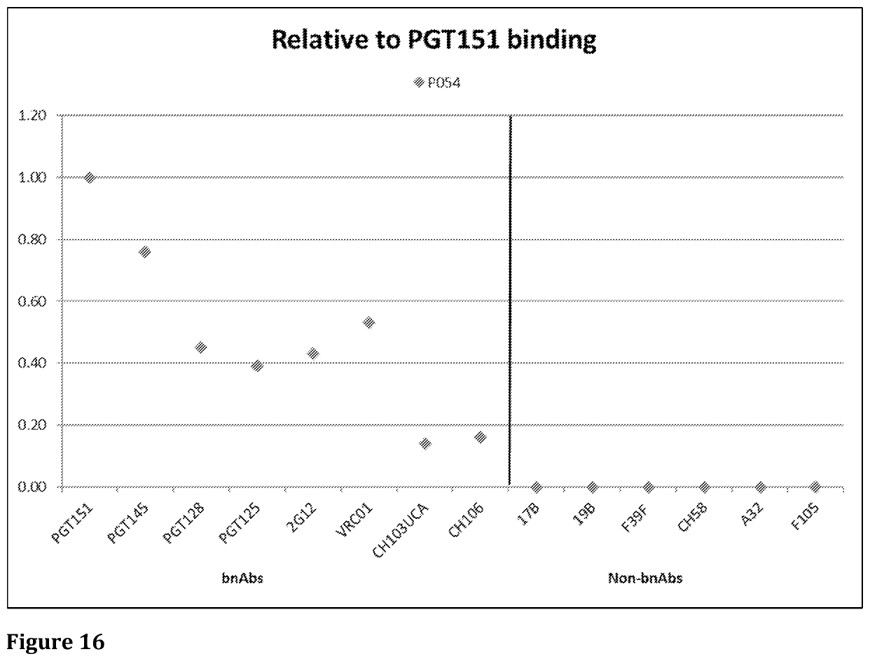

FIG. 13 shows SDS-PAGE results of purification of the CH505 TF stabilized SOSIP trimer by Method 3 and illustrates the high increase in purity of the Phenyl Sepharose product pool over the starting harvest. FIG. 14 shows the SEC profile of the purified trimer. FIG. 15 shows trimer homogeneity using nsEM, and FIG. 16 shows binding of purified trimer in the Phenyl product pool to bnAbs and not to non-neutralizing antibodies

Purification of a Second SOSIP Trimer (Trimer-2) Using Combination Chromatography Columns.

To determine if non-affinity combination chromatography column purification methods could be applied to additional HIV-1 Env SOSIP trimers, a second SOSIP trimer Trimer-2 was purified with Method 2 (FIG. 7).

The purification of the Trimer-2 by Method 2 used TFF to selectively retain HIV-1 Env and prepare clarified harvest for the initial chromatography step. Clarified harvest was ultrafiltered and diafiltered through a membrane with specific pore size capable of separating trimer Env and smaller product variants. The membrane was constructed from modified polyethersulfone (mPES) with 300 kD molecular weight cutoff. The first chromatography step used in Method 2, FIG. 17, used Toyopearl 750F to bind and elute the target molecule into a single bulk fraction. Toyopearl NH2 750F is a salt tolerant anion exchanger with a wide pH range for loading. For HIV-1 Env, it is recommended to bind at a unique pH for a typical anion exchanger. HIV-1 Env was bound to the resin at pH 7.2 and low ionic strength (30 mM NaCl). While bound to the column, HIV-1 Env was washed with equilibration buffer. HIV-1 Env eluted with increasing salt. Table 7 depicts the Toyopearl NH2 750F step-by-step operation including post-product cleaning steps. Toyopearl NH2 750F eluted HIV-1 Env enriched for trimeric gp140.

TABLE 7
Toyopearl NH2 750F step by step operating instructions

| | | Column | Flow |
|---|---|---|---|
| | | Volume | Rate |
| Step Name | Buffer | (CV) | (cm/hr) |
| Equilibration | 20 mM HEPES (Acid), | 5 | 200 |
| | 30 mM NaCl pH 7.2 | | |
| Load | pH 7.2 | 6 | 100 |
| Wash 1 | 20 mM HEPES (Acid) | 5 | 100 |
| | 30 mM NaCl pH 7.2 | | |
| Linear Gradient | 20 mM NaHEPES, | 15 CV | 200 |
| Elution | 0.52 M NaCl, pH 7.2 - | Linear | |
| Collection | pH 7.220 mM NaHEPES, | Gradient | |
| | 2 M NaCl pH 7.2 | | |
| Strip | 20 mM Acetate, 3 M | 4 | 200 |
| | NaCl, pH 5 | | |
| Sanitization | 0.5 M NaOH | 5 | 100 |
| Rinse | 20 mM HEPES (Acid), | 5 | 200 |
| | 30 mM NaCl pH 7.2 | | |
| Storage | 20% Ethanol | 5 | 200 |

The second chromatography step in the purification of the Trimer-2 trimer by Method 2 used Capto Phenyl High Sub (HIC) resin to bind and elute the target molecule into a single bulk fraction. NH2 Eluate was diluted with 2.4M ammonium sulfate to a final ammonium sulfate concentration of 1.8M and loaded onto the HIC resin at pH 7.2. While bound to the column, HIV-1 Env was washed with equilibration buffer similar to the loading pH and conductivity conditions. HIV-1 Env was eluted with decreasing salt. Table 8 depicts the executed HIC step-by-step operation including pre-product and post-product cleaning steps.

TABLE 8
Capto Phenyl step by step operating instructions

| | | Column | Flow |
|---|---|---|---|
| | | Volume | Rate |
| Step Name | Buffer | (CV) | (cm/hr) |
| Equilibration | 20 mM HEPES (Acid), | 5 | 200 |
| | 1.8 M Ammonium | | |
| | Sulfate, pH 7.2 | | |
| Load | 1.8 M Ammonium | 30 mL UFDF | 100 |
| | Sulfate pH 7.2 | Ret | |
| Wash 1 | 20 mM HEPES (Acid), | 5 | 100 |
| | 1.8 M Ammonium | | |
| | Sulfate, pH 7.2 | | |
| Eluate | 20 mM NaHEPES, pH | 18 CV | 200 |
| Collection | 7.2 | Linear Gradient | |
| | | (0-100% B1) | |
| Strip | MilliQ H2O | 4 | 200 |
| Sanitization | 0.5 M NaOH | 5 | 100 |
| Rinse | 20 mM HEPES (Acid), | 5 | 200 |
| | 30 mM NaCl pH 7.2 | | |
| Storage | 20% Ethanol | 5 | 200 |

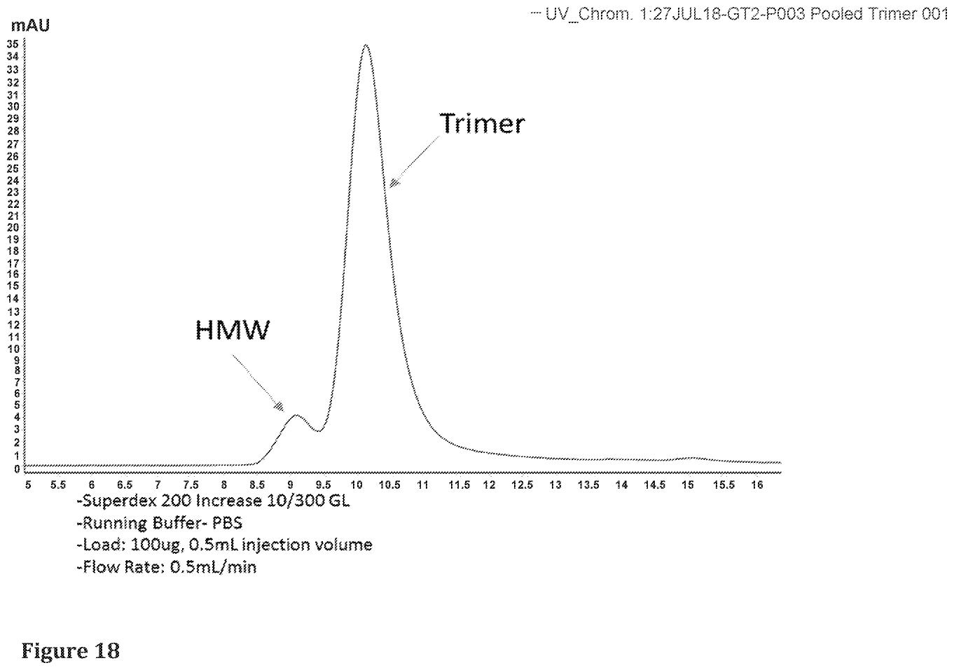

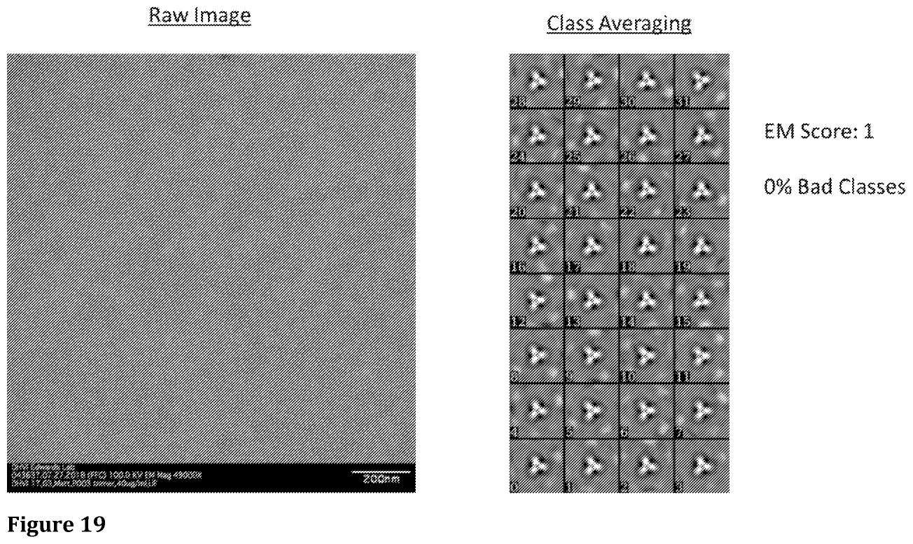

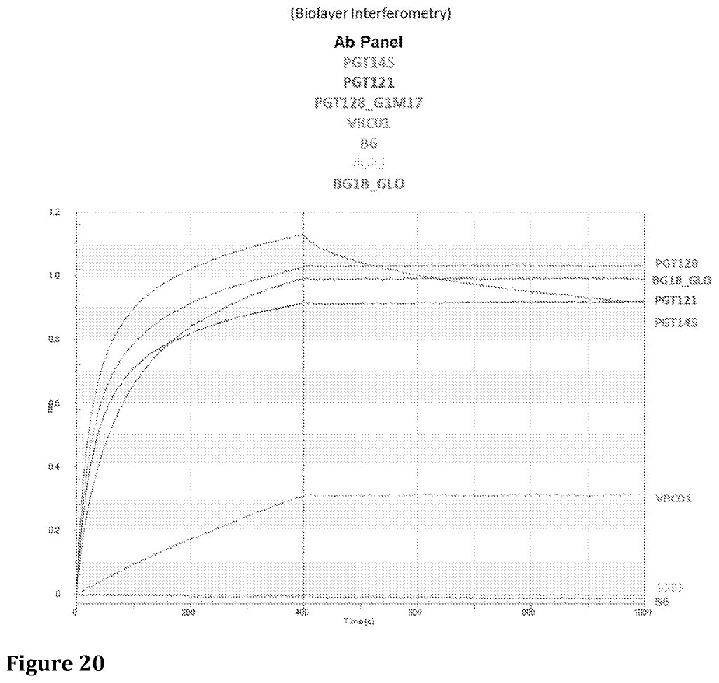

FIG. 17 shows SDS-PAGE results of purification of Trimer-2 SOSIP trimer by Method 2 and shows high gp140 purity in the Capto Phenyl product pool, FIG. 18 shows the SEC profile of the Phenyl product pool indicating uniform, trimeric gp140. FIG. 19 shows trimer homogeneity using nsEM, and FIG. 20 shows binding of purified Trimer-2 to bnAbs (PGT145, PGT121, PGT128, VRC01, and BG18_GLO) but not to non-neutralizing antibodies (B6 and 4025).

Exemplary GMP Purification Process for HIV-1 Env SOSIP Trimers

An exemplary purification process for GMP manufacture of HIV-1 Env SOSIP trimers is shown in FIGS. 21A-B. This process includes process steps that remove product and process related impurities (chromatography steps) and dedicated steps that ensure product safety (i.e., viral inactivation, viral filtration, bioburden reduction filtration). All steps in this process are GMP compliant and are scalable to full commercial manufacturing.

Purification Method for Recombinant SOSIP Trimer in Nanoparticles

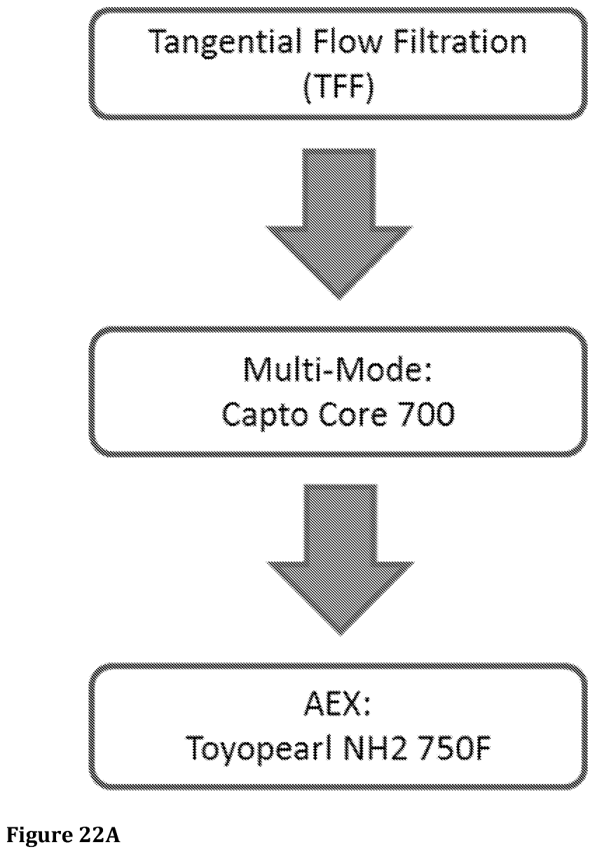

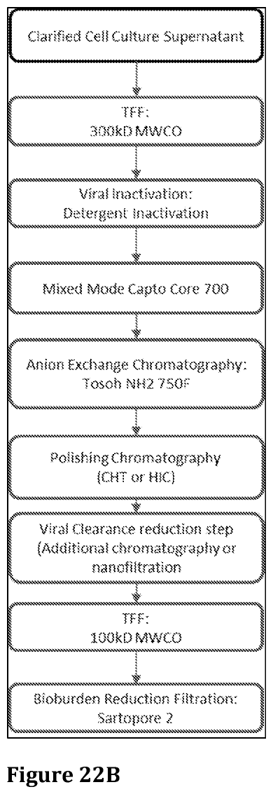

Using a Trimer-2 envelope-ferritin nanoparticle (NP-2) recombinantly expressed in CHO-DG44 cells, we developed an alternative purification process to the standard currently used in the field. Affinity and size exclusion chromatography were replaced with multi-mode and ion-exchange chromatography. In the simplest version the isolation of intact, uniform NP was accomplished in three purification steps (FIG. 22A).

The initial purification operation in NP purification method (FIGS. 22A-B) used TFF to selectively retain HIV-1 Env NP and prepare clarified harvest for the initial chromatography step. Clarified harvest was ultrafiltered and diafiltered through a membrane with specific pore size capable of separating Env NP and small product variants. The membrane was constructed from modified polyethersulfone (mPES) with 300 kD molecular weight cutoff.

Capto Core 700 is a GMP compliant, scalable multi-modal resin produced by GE Life Sciences. The base matrix for this resin is highly cross-linked agarose that is base stable for easy cleanability and can withstand high pressure that enables decreased run times. This resin exhibits multimodal separation modalities with separation occurring by size, ionic properties and hydrophobicity. The Capto Core 700 is composed of a ligand-activated core and inactive shell; the inactivated shell excludes molecules that are larger than the apparent size of globular 700 kD protein. Molecules smaller than a 700 kD globular protein can enter the pore and bind the mix-mode ligand-activated core, the active ligand is an octlyamine that exhibits both hydrophobic and anion exchange modes of interaction. HIV envelope ferritin nanoparticles have been shown to have low molecular weight product-related impurities, these can include free gp140 subunits (i.e., gp120 and/or gp41), free gp140 monomer, free gp140 trimer and nanoparticles that are not completely assembled. Low molecular weight impurities are small enough to pass through the inactivated shell and bind the ligand core, while fully assembled nanoparticle cannot pass through the inactivated shell and thus can be collected the column flow through fraction.

FIGS. 22A and 22B illustrate the purification process used in the NP purification method. The initial chromatography step used Capto Core 700 to isolate intact nanoparticle in the column flow through while binding low molecular weight impurities to the column. Buffer exchanged and concentrated clarified harvest was ran over a Capto Core 700 column and washed from the column with 20 mM HEPES, 170 mM NaCl, pH7.2, the low molecular weight impurities bound the column and were stripped off the column with 2M NaCl. Details of Capto Core 700 operation are detailed in Table 9.

TABLE 9
Step by step process of Capto Core 700
column chromatography for Nanoparticle
purification

| | | Column | Flow |
|---|---|---|---|
| | | Volume | Rate |
| Step Name | Buffer | (CV) | (cm/hr) |
| Equilibration | 20 mM HEPES, | 5 | 200 |
| | 170 mM NaCl | | |
| | pH 7.2 | | |
| Load | UFDF Harvest | 13 | 100 |
| Wash | 20 mM HEPES, | 5 | 100 |
| | 170 mM NaCl | | |
| | pH 7.2 | | |
| Strip | 50 mM Tris, | 4 | 200 |
| | 2 M NaCl | | |
| Sanitization | 1 N NaOH in | 5 | 100 |
| | 30% IPA | | |
| Rinse | 20 mM HEPES, | 5 | 200 |
| | 170 mM NaCl | | |
| | pH 7.2 | | |
| Storage | 20% Ethanol | 5 | 200 |

The second chromatography step in NP purification method used Toyopearl NH2 750F to bind the nanoparticle and selectively elute the nanoparticle with a linear gradient of increasing ionic strength. Capto Core 700 flow through from chromatography step 1 was loaded onto an equilibrated NH2 column and then impurities were washed from the column with 20 mM HEPES, 170 mM NaCl pH 7.2. A linear gradient of increasing sodium chloride concentration was then used to elute the target nanoparticle. Step by step operating instructions for the Toyopearl NH2 750F chromatography step is depicted in Table 10. The NH2 product pool was buffer exchanged into an appropriate buffer for analysis by SDS-PAGE, SEC, NS-EM and ELISA.

TABLE 10
Step by Step operating instructions for NH2 750F for
Nanoparticles

| | | Column | Flow |
|---|---|---|---|
| | | Volume | Rate |
| Step Name | Buffer | (CV) | (cm/hr) |
| Equilibration | 20 mM HEPES, 170 | 5 | 200 |
| | mM NaCl pH 7.2 | | |
| Load | pH 7.2 | ~35 ml | 100 |
| Wash 1 | 20 mM HEPES, 170 | 5 | 100 |
| | mM NaCl pH 7.2 | | |
| Eluate | 20 mM HEPES, 170 | 10 CV Linear | 200 |
| Collection | mM NaCl pH 7.2 - | Gradient | |
| | 20 mM HEPES, 2 | (0-100% B1) | |
| | M NaCl pH 7.2 | | |
| Strip | 50 mM Tris, 2 M | 4 | 200 |
| | NaCl | | |
| Sanitization | 0.5 M NaOH | 5 | 100 |
| Rinse | 20 mM HEPES, 170 | 5 | 200 |
| | mM NaCl pH 7.2 | | |
| Storage | 20% Ethanol | 5 | 200 |

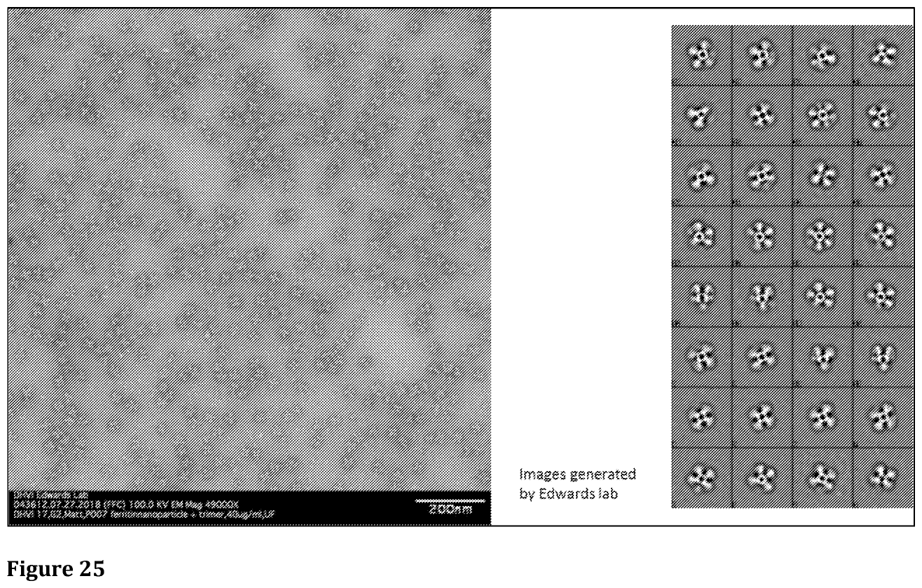

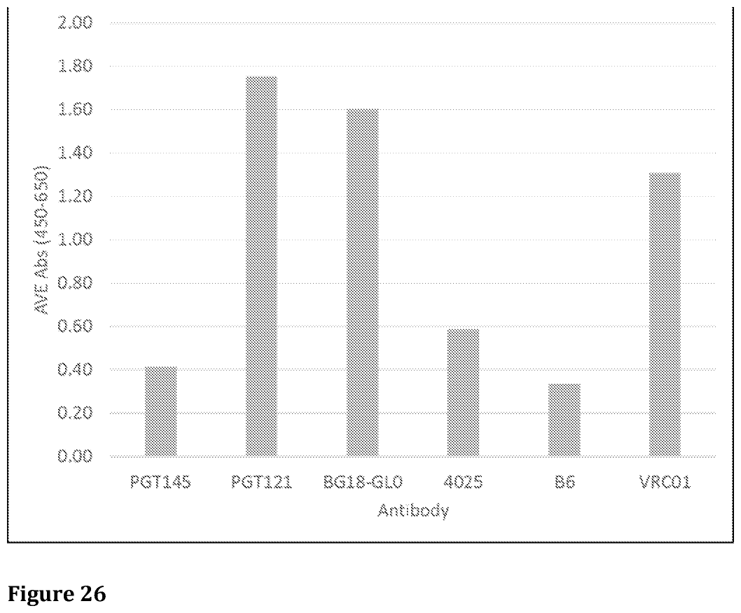

FIG. 23 shows SDS-PAGE results of purification of the NP-2 and shows a significant increase in purity for the Toyopearl NH2 750F product pool over the starting harvest material, FIG. 24 shows a single, uniform population of NP-2 by SEC. FIG. 25 shows NP-2 homogeneity using NS-EM, and FIG. 26 shows the expected binding profile of purified NP-2 to bnAbs (PGT145, PGT121, BG18-GL0, and VRC01) and non-neutralizing antibodies (4025 and B6).

Purification of a Second SOSIP Trimer Nanoparticle (NP-CONS) with Combined Non-Affinity Column Chromatography Methods.

To demonstrate if non-affinity purification methods could be applied to additional HIV-1 Env SOSIP trimer nanoparticles, a second nanoparticle, SOSIP Env NP (“NP-CONS” group M consensus Env CON-S SOSIP trimer as a ferritin fusion protein) was purified with a slightly modified version of the process used for NP-2 (FIGS. 22A-B).

The purification of the CON-S Env NP was similar to the method used for purification of the NP-CONS (FIGS. 22A-B), with the only alteration in the process was the composition in the DF buffer in TFF and the EQ buffers of the chromatography steps. Step by step instructions for the Capto Core 700 and NH2 750F can be seen in Table 11 and Table 12, respectively.

TABLE 11
Step by step process of Capto Core 700 for
molecule 2 (NP-2)

| | | Column | Flow |
|---|---|---|---|
| | | Volume | Rate |
| Step Name | Buffer | (CV) | (cm/hr) |
| Equilibration | 20 mM | 5 | 200 |
| | HEPES, | | |
| | 100 mM NaCl | | |
| | pH 7.2 | | |
| Load | UFDF Harvest | 20 | 100 |
| Wash | 20 mM | 5 | 100 |
| | HEPES, | | |
| | 100 mM NaCl | | |
| | pH 7.2 | | |
| Strip | 50 mM Tris, | 4 | 200 |
| | 2 M NaCl | | |
| Sanitization | 1 N NaOH in | 5 | 100 |
| | 30% IPA | | |
| Rinse | 20 mM | 5 | 200 |
| | HEPES, | | |
| | 170 mM NaCl | | |
| | pH 7.2 | | |
| Storage | 20% Ethanol | 5 | 200 |

TABLE 12
Step by step process of NH2 750F for
molecule 2 (NP-2)

| | | Column | Flow |
|---|---|---|---|
| | | Volume | Rate |
| Step Name | Buffer | (CV) | (cm/hr) |
| Equilibration | 20 mM | 5 | 200 |
| | HEPES, | | |
| | 100 mM NaCl | | |
| | pH 7.2 | | |
| Load | pH 7.2, | 20 | 100 |
| | 150 mM NaCl | | |
| Wash 1 | 20 mM | 5 | 100 |
| | HEPES, | | |
| | 100 mM NaCl | | |
| | pH 7.2 | | |
| Eluate | 20 mM | 20 | 200 |
| Collection | NaHEPES, 2 M | | |
| | NaCl pH 7.2 | | |
| Strip-1 | 50 mM | 4 | 200 |
| | Acetate, 3 M | | |
| | NaCl | | |
| Sanitization | 0.5M NaOH | 5 | 100 |
| Rinse | 20 mM | 5 | 200 |
| | HEPES, | | |
| | 150 mM NaCl | | |
| | pH 7.2 | | |
| Storage | 20% Ethanol | 5 | 200 |

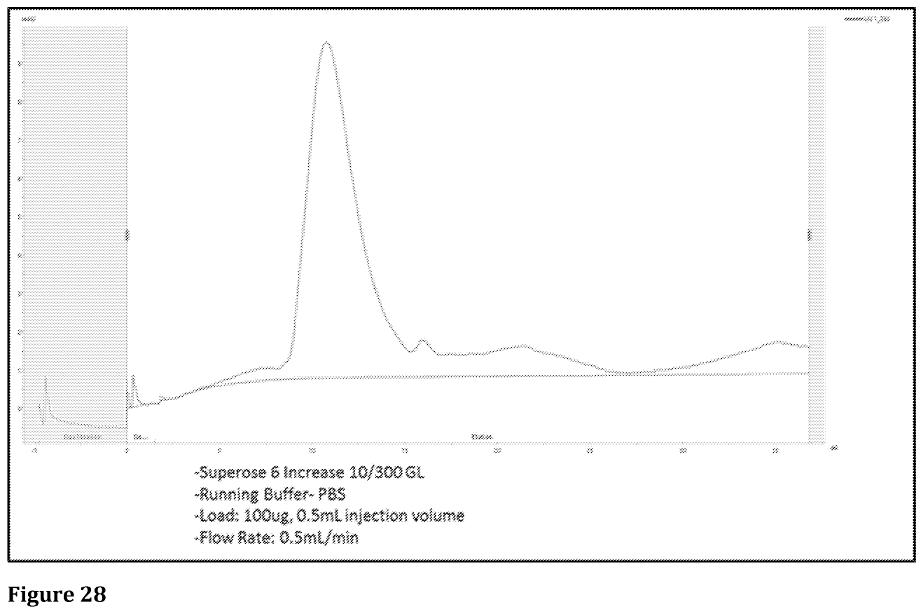

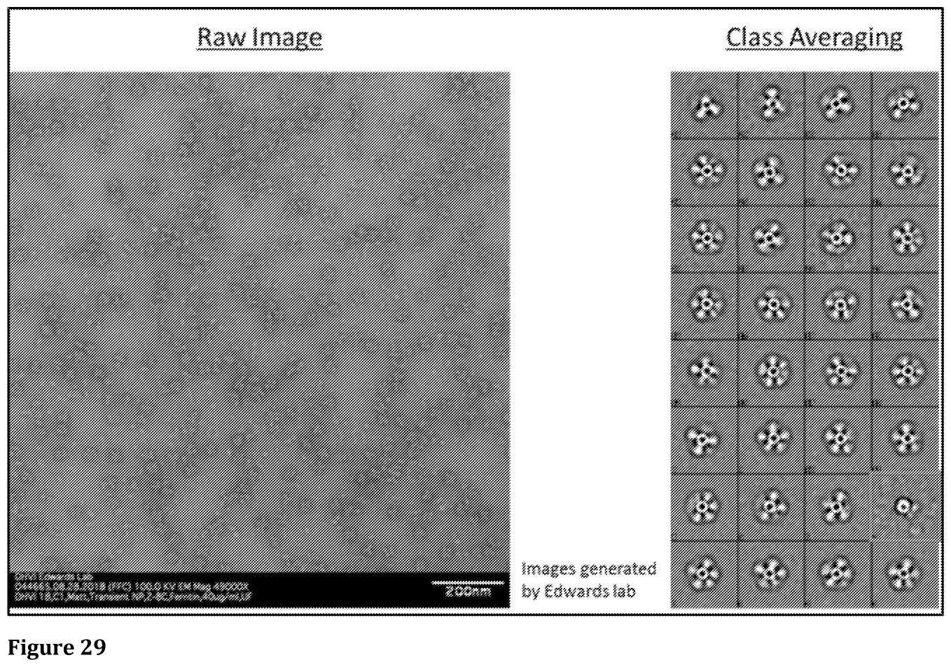

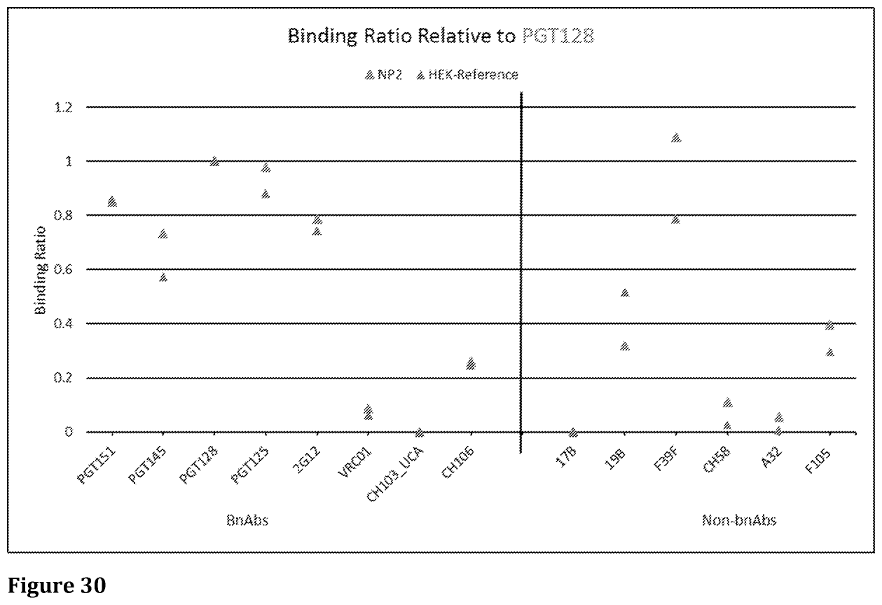

FIG. 27 shows SDS-PAGE results of purification of the NP-CONS and shows a significant increase in purity for the Toyopearl NH2 750F product pool over the starting harvest material, FIG. 28 shows a single, uniform population of NP-CONS by SEC. FIG. 29 shows NP-CONS homogeneity using NS-EM, and FIG. 30 shows the expected binding profile of purified NP-CONS to bnAbs (PGT151, PGT145, PGT128, PGT125, 2G12, VRC01, and CH106) and non-neutralizing antibodies (17B, 19B, F393F, CH58, A32, and F105).

Discussion

In this example we describe methods for purification of soluble HIV-1 Env trimers as well as HIV-1 Env nanoparticles that do not require the use of antibody affinity chromatography. For soluble trimers, three methods using an initial TFF step followed by two chromatography steps Toyopearl NH2 750F and CHT Type 1 40 μm for Method 1, Toyopearl NH2 750F and Capto Phenyl for Method 2, and Capto DeVirS and Phenyl Sepharose FF for Method 3. For Env nanoparticles TFF followed by Capto Core 700 and Toyopearl NH2 750 can be used.

HIV-1 Env trimers and nanoparticles can be separated based on size using size exclusion chromatography (SEC) but there are limitations to SEC when used to produce large quantities of protein. Operationally, commonly used size exclusion resins require high operating pressure and many manufacturers do not have the equipment and personnel to safely execute. The high pressure requires specialized, costly equipment and flow rates are prohibitively slow. Additionally, size exclusion chromatography is difficult to execute consistently at larger scales due to inconsistency in column packing and column loading. The proportion of load volume to column volume must be controlled for consistent separation. SEC manufacturing challenges and process control challenges are associated with variable product (lack of robustness) and commonly require fractionation with immediate in-process analytics. Size exclusion chromatography may be used for small batches of material (11); however, due to the reasons described, SEC is not suitable for manufacturing quantities required for worldwide supply.

The current standard in the field that includes antibody affinity chromatography and size exclusion chromatography (see Dey et al Biotechnol Bioeng. 2018; 115(4):885-99, and (19)). The Env trimer purification methods described herein offer alternative approaches for the purification of properly folded trimers. Isolation of properly folded Env trimer is accomplished using commercially available, off the shelf chromatography resins that are scalable to full commercial scale, simple to implement in GMP production, and it is hypothesized that can be used for different classes of Env trimers.

The Env trimer NP purification process described herein offers an alternative approach for the isolation of fully formed self-assembling Env trimer NP than the current standard in the field for the purification of self-assembling nanoparticles that includes antibody or lectin affinity and often size exclusion chromatography (20, 21). The purification of fully formed Env Trimer NP can be accomplished using commercially available, off the shelf chromatography resins that are not limited by the high cost, lack of cleanability, and high specificity of affinity resins. This non-affinity method is fully scalable to commercial scale and can be used to purify other HIV Env NPs.

In summary, we have described combined non-affinity column chromatography schema for purification of SOSIP trimers and for Env NP multimers. These methods should allow for scale up of trimeric or multimeric Env immunogens in GMP Env production.

### References for Example 1

1. Mascola J R, Haynes B F. HIV-1 neutralizing antibodies: understanding nature's pathways. Immunological reviews. 2013; 254(1):225-44.
2. Sanders R W, Derking R, Cupo A, Julien J P, Yasmeen A, de Val N, et al. A next-generation cleaved, soluble HIV-1 Env trimer, BG505 SOSIP.664 gp140, expresses multiple epitopes for broadly neutralizing but not non-neutralizing antibodies. PLoS pathogens. 2013; 9(9):e1003618.
3. Go E P, Ding H, Zhang S, Ringe R P, Nicely N, Hua D, et al. Glycosylation Benchmark Profile for HIV-1 Envelope Glycoprotein Production Based on Eleven Env Trimers. Journal of virology. 2017; 91(9).
4. Haynes B F, Kelsoe G, Harrison S C, Kepler T B. B-cell-lineage immunogen design in vaccine development with HIV-1 as a case study. Nature biotechnology. 2012; 30(5):423-33.
5. LaBranche C C, McGuire A T, Gray M D, Behrens S, Zhou T, Sattentau Q J, et al. HIV-1 envelope glycan modifications that permit neutralization by germline-reverted VRC01-class broadly neutralizing antibodies. PLoS pathogens. 2018; 14(11):e1007431.
6. Liao H X, Lynch R, Zhou T, Gao F, Alam S M, Boyd S D, et al. Co-evolution of a broadly neutralizing HIV-1 antibody and founder virus. Nature. 2013; 496(7446):469-76.
7. Sok D, Briney B, Jardine J G, Kulp D W, Menis S, Pauthner M, et al. Priming HIV-1 broadly neutralizing antibody precursors in human Ig loci transgenic mice. Science. 2016; 353(6307):1557-60.
8. Williams W B, Zhang J, Jiang C, Nicely N I, Fera D, Luo K, et al. Initiation of HIV neutralizing B cell lineages with sequential envelope immunizations. Nat Commun. 2017; 8(1):1732.
9. Medina-Ramirez M, Garces F, Escolano A, Skog P, de Taeye S W, Del Moral-Sanchez I, et al. Design and crystal structure of a native-like HIV-1 envelope trimer that engages multiple broadly neutralizing antibody precursors in vivo. The Journal of experimental medicine. 2017; 214(9):2573-90.
10. Dosenovic P, von Boehmer L, Escolano A, Jardine J, Freund N T, Gitlin A D, et al. Immunization for HIV-1 Broadly Neutralizing Antibodies in Human Ig Knockin Mice. Cell. 2015; 161(7):1505-15.
11. Dey A K, Cupo A, Ozorowski G, Sharma V K, Behrens A J, Go E P, et al. cGMP production and analysis of BG505 SOSIP.664, an extensively glycosylated, trimeric HIV-1 envelope glycoprotein vaccine candidate. Biotechnol Bioeng. 2018; 115(4):885-99.
12. Verkoczy L. Humanized Immunoglobulin Mice: Models for HIV Vaccine Testing and Studying the Broadly Neutralizing Antibody Problem. Adv Immunol. 2017; 134:235-352.
13. Saunders K O, Nicely N I, Wiehe K, Bonsignori M, Meyerhoff R R, Parks R, et al. Vaccine Elicitation of High Mannose-Dependent Neutralizing Antibodies against the V3-Glycan Broadly Neutralizing Epitope in Nonhuman Primates. Cell reports. 2017; 18(9):2175-88.
14. Sliepen K, Ozorowski G, Burger J A, van Montfort T, Stunnenberg M, LaBranche C, et al. Presenting native-like HIV-1 envelope trimers on ferritin nanoparticles improves their immunogenicity. Retrovirology. 2015; 12:82.
15. Zhang R, Verkoczy L, Wiehe K, Munir Alam S, Nicely N I, Santra S, et al. Initiation of immune tolerance-controlled HIV gp41 neutralizing B cell lineages. Science translational medicine. 2016; 8(336):336ra62.
16. Group I E W. Viral Safety Evaluation of Biotechnology Products Derived from Cell Lines of Human or Animal Origin Q5A (R1) ICH.org 1999.
17. Crublet E, Andrieu J P, Vives R R, Lortat-Jacob H. The HIV-1 envelope glycoprotein gp120 features four heparan sulfate binding domains, including the co-receptor binding site. The Journal of biological chemistry. 2008; 283(22):15193-200.
18. Harrop H A, Rider C C. Heparin and its derivatives bind to HIV-1 recombinant envelope glycoproteins, rather than to recombinant HIV-1 receptor, CD4. Glycobiology. 1998; 8(2): 131-7.
19. Ringe R P, Yasmeen A, Ozorowski G, Go E P, Pritchard L K, Guttman M, et al. Influences on the Design and Purification of Soluble, Recombinant Native-Like HIV-1 Envelope Glycoprotein Trimers. Journal of virology. 2015; 89(23):12189-210.
20. He L, de Val N, Morris C D, Vora N, Thinnes T C, Kong L, et al. Presenting native-like trimeric HIV-1 antigens with self-assembling nanoparticles. Nat Commun. 2016; 7:12041.
21. Kanekiyo M, Wei C J, Yassine H M, McTamney P M, Boyington J C, Whittle J R, et al. Self-assembling influenza nanoparticle vaccines elicit broadly neutralizing H1N1 antibodies. Nature. 2013; 499(7456): 102-6.

### Example 2: Production Scale

Examples 1 used material from a small scale cell line expression and purification was conducted on a small scale. These are expected to be fully scalable for large scale cell line production and purification suitable for commercial GMP manufacturing processes. In some non-limiting embodiments, the purification process can be scaled to full commercial production scales, including but not limited to 50 L, 100 L, 150 L, 200 L, 250 L, 300 L, 350 L, 400 L, 450 L, 500 L, 550 L, 600 L, 650 L, 700 L, 750 L, 800 L, 850 L, 900 L, 950 L, 1000 L, 1500 L, 2000 L, 2500 L, 3000 L, 3500 L, 4000 L, 4500 L, 5000 L, 6000 L, 7000 L, 8000 L, 9000 L, 10000 L, 15000 L, up to 20,000 L bioreactor scale.

In large-scale production, additional steps will be added to address requirements for GMP production; these may include, but not limited to, viral removal steps to give added viral clearance, sterile filtration to reduce bioburden, and a final formulation step to buffer exchange drug substance into appropriate buffer for the clinic.

Methods are suitable for stable cell lines and also for transiently transfected cell lines.

### Example 3

Animal models, e.g. mice, rabbits, guinea pigs, non-human primates, will be used to test the immunogenicity of the material purified by the instant methods.

GMP produced material will be used in clinical trials.

### Example 4: ConS-NP Design and Sequences

### Example 4A: ConS Envelope Designs as panbnAb Immunogens

To cover the diversity of HIV-1 isolates that circulate globally a consensus envelope was derived from all group M HIV-1 isolates available at the time (Liao H X et al Virology 2006; see also U.S. Pat. No. 8,071,107 and all parent applications and applications claiming priority to) called CON-S. To induce neutralizing antibodies it is hypothesized that the immunogen should mimic the native, fusion-competent envelope on viruses. To create stable mimics of the HIV-1 Env CON-S we created SOSIP gp140s. The SOSIP gp140 was stabilized by introducing BG505 amino acids into the gp120 and gp41 regions as we have described previously (Saunders K O, Vercokzy L et al., Cell Reports, Volume 21, Issue 13, P3681-3690, Dec. 26, 2017). The Env was further stabilized by introducing a disulfide bond between amino acids at position 201 and 433 (Do-Kwon Y et al., Nat Struct Mol Biol., 2015 July, 22(7):522-31).

The CON-S sequence was furthered optimized to bind to antibodies that target the V3-glycan broadly neutralizing site by removing glycans that were determined in neutralization assays to inhibit V3-glycan antibody binding and neutralization. We hypothesize that broadly neutralizing antibody precursors have low affinity for HIV-1 Env which necessitates reducing steric barriers and glycosylation changes that hinder precursor antibody binding. In neutralization assays we identified that glycans attached between N131 and N141 prohibited neutralization by precursor antibodies that were developing neutralization breadth. Thus, to improve binding to the V3-glycan site on CON-S stabilized gp140 SOSIPs we removed glycosylation sites at 130, 135, 138, and 141 by substituting asparagine for naturally occurring amino acids identified in the HIV-1 sequence database. The mutant Env contained N130D, N135K, N138S, and N1415 mutations. Using mass spectrometry we verified that the glycans at 295, 301, and 332 were still the high mannose glycans preferentially bound by broadly neutralizing antibodies PGT128, PGT124, PGT135, DH270, BF520, and BG18. While removal of the V1 glycans may allow better binding to Env, the affinity for Env may be low for certain V3-glycan bnAb precursors. It has been shown that B cell receptors recognize low affinity antigen better when it is presented on a surface rather than free in solution (Batista F and Neuberger M, J EMBO, 2000, 19(4):513-520). Thus we took the Env and arrayed it on the surface of a ferritin nanoparticle so that 8 copies of the CON-S SOSIP trimer could be displayed to B cells to maximize avidity of the BCR:SOSIP interaction. In total, a stabilized soluble HIV-1 Env trimer was derived from a consensus of group M and inhibitory glycans were removed to promote V3-glycan bnAb precursor binding. The optimized Env was arrayed on ferritin nanoparticles to enhance avidity between Env and B cell receptors.

The removal of 4 glycans in the V1 loop was hypothesized to permit binding of Env to unmutated bnAb precursos to initiate bnAb lineages. To select the bnAb intermediate antibodies within a lineage that are acquiring the ability to bind to multiple native Envs, we created a CON-S SOSIP Env trimers that added back the N130 and N135 glycosylation sites. This Env lacks glycosylation sites at 138 and 141 functions to select the antibodies that bind to Env with the correct mode to accommodate the N130 and N135 glycans. In a sequential vaccine this Env would be administered after the 4 glycan deleted Env but before the wildtype Env so that glycans are sequentially added back to the Env to select the small population of B cells that recognize the V3-glycan site with the correct binding orientation.

### Example 4B. Glycan-Optimized Trimeric HIV-1 Envelope Elicits Glycan-Dependent Autologous Tier 2 Neutralizing Antibodies in Rhesus Macaques

Introduction

Vaccine elicitation of broadly neutralizing antibodies (bnAbs) against HIV-1 has yet to be achieved. The target of bnAbs is HIV-1 envelope (Env) which is shielded by host glycans that hinder its recognition by antibodies. During natural infection, bnAbs develop that recognize the glycans and peptide proximal to the third variable region (V3-glycan). These glycan-dependent antibodies are protective in nonhuman primate models of HIV-1 infection. We previously observed that reactivity with Env was enhanced for V3-glycan bnAbs when the Env was enriched for Man9GlcNAc2 glycans or when V1 glycans were removed. We hypothesize that glycan-dependent bnAbs can be induced in primates with a vaccine if the immunogens are optimized to engage V3-glycan bnAb precursors and subsequently select for B cells within those lineages that are developing neutralization breadth.

The scientific premises of the non-human primate study (NHP145) is

1. V3 glycan precursors prefer kif treated Env.
2. A multimer is needed to activate the germline precursors because the affinity is so low.
3. V3 glycan precursors have to learn to accommodate processed glycans one at a time.

Methods

Recombinant trimeric HIV-1 CON-S Env was made as a SOSIP trimer and arrayed on ferritin nanoparticles. To enrich for Man9GlcNAc2 some Env were treated with kifunensine (kif). Trimer formation was determined by negative stain electron microscopy (EM). Antigenicity of the Envs was determined by Bio-layer interferometry. Four rhesus macaques were vaccinated 6 times with a series of HIV-1 Env glycosylation variants optimized to be antigenic for V3-glycan bnAbs. Binding and neutralizing antibodies were measured by ELISA and the TZM-bl assay respectively.

Conclusions:
1. Modified CON-S nanoparticles bind to the precursors of V3-glycan and V1V2 glycan bnAbs.
2. Multimerization of HIV-1 Env induces more durable antibody responses than free trimer.
3. Neutralizing antibody responses show that vaccination can elicit glycan-dependent neutralizing antibodies against the same Asn301 glycan targeted by bnAbs.

### References

Stewart-Jones et al. Cell. 2016 May 5; 165(4):813-26. doi: 10.1016/j.cell.2016.04.010. Epub 2016 Apr. 21.
Saunders et al., 2017 Cell Rep., 18 (2017), pp. 2175-2188.

### Example 4C: Table 13. Con-S Sequences

| Plasmid number | Protein name |
|---|---|
| HV1301184 (SEQ ID NO: 1) | CON-S.6R.SOSIP.664 (SEQ ID NO: 24) |
| HV1301185 (SEQ ID NO: 2) | CON-S.6R.DS.SOSIP.664 (SEQ ID NO: 25) |
| HV1301186 (SEQ ID NO: 3) | CON-S.6R.SOSIP.664.v3.1 (SEQ ID NO: 26) |
| HV1301187 (SEQ ID NO: 4) | CON-S.6R.SOSIP.664.v4.1 (SEQ ID NO: 27) |
| HV1301188 (SEQ ID NO: 5) | CON-S.6R.SOSIP.664.v4.2 (SEQ ID NO: 28) |
| HV1301257 (SEQ ID NO: 6) | CON-Schim.6R.SOSIP.664_avi (SEQ ID NO: 29) |
| HV1301258 (SEQ ID NO: 7) | CON-Schim.6R.DS.SOSIP.664_avi (SEQ ID NO: 30) |
| HV1301259 (SEQ ID NO: 8) | CON-Schim.6R.SOSIP.664v4.1_avi (SEQ ID NO: 31) |
| HV1301260 (SEQ ID NO: 9) | CON-Schim.6R.SOSIP.664v4.2_avi (SEQ ID NO: 32) |
| HV1301639_avi (SEQ ID | CON-Schim.6R.DS.SOSIP.664_N130D_N135K_avi (SEQ ID NO: 40) |
| NO: 17) | |
| HV1301640_avi (SEQ ID | CON-Schim.6R.DS.SOSIP.664_1\1138S_N1415_avi (SEQ ID NO: 41) |
| NO: 18) | |
| HV1301641_avi (SEQ ID | CON-Schim.6R.DS.SOSIP.664_N130D_N135K_N138S_N141S_avi |
| NO: 19) | (SEQ ID NO: 42) |
| HV1301613 (SEQ ID NO: 20) | CON-Schim.6R.DS.SOSIP.664v4.1_OPT (SEQ ID NO: 43) |
| HV1301521_ferritin (SEQ ID | CON-Schim.6R.DS.SOSIP.664_OPT_N130D_N135K_N138S_N141S_ferritin |
| NO: 10) | (SEQ ID NO: 33) |
| HV1301521 (SEQ ID NO: 11) | CON-Schim.6R.DS.SOSIP.664_OPT_N130D_N135K_N138S_N141S |
| | (SEQ ID NO: 34) |
| HV1301405 N138S_N141S | CON-Schim.6R.DS.SOSIP.664_OPT_N138S_N141S (SEQ ID NO: 35) |
| (SEQ ID NO: 12) | |
| HV1301405 (SEQ ID NO: 13) | CON-Schim.6R.DS.SOSIP.664_OPT (SEQ ID NO: 36) |
| HV1301258 N301A (SEQ | CON-Schim.6R.DS.SOSIP.664_N301A_avi (SEQ ID NO: 37) |
| ID NO: 14) | |
| HV1301258_N332A (SEQ | CON-Schim.6R.DS.SOSIP.664_N332A_avi (SEQ ID NO: 38) |
| ID NO: 15) | |
| HV1300111_avi_N137A | CON-Sgp140CFI_avi_N137A (SEQ ID NO: 44) |
| (SEQ ID NO: 21) | |
| HV1300111_avi_N141A | CON-Sgp140CFI_avi_N141A (SEQ ID NO:45) |
| (SEQ ID NO: 22) | |
| HV1300111_avi_V1_4Q | |
| (SEQ ID NO: 23) | CON-Sgp140CFI_avi_V1_4Q (SEQ ID NO: 46) |
| HV1301521_c_sorta (SEQ | CON- |
| ID NO: 16) | Schim.6R.DS.SOSIP.664_OPT_N130D_N135K_N138S_N141S_C-SortaseA |
| | (SEQ ID NO: 39) |

Sequences from Example 4 are shown in FIG. 31.

### Example 5: Large Scale Production and Purification of Envelope Trimer

This example describes the downstream process for the purification of CH505TF4.1 SOSIP Trimer (also referred to as CH505 T/F4.1 or TF4.1). See FIG. 32 for sequence, and FIG. 21B for the complete process steps. The TF4.1 downstream process consists of eight unique unit operations. The process described in FIGS. 21A-B is scale independent. The process was carried out with harvested material from CHO cells grown in 50 L and 200 L bioreactors.

TF4.1 is a cleaved, soluble, secreted trimeric gp140 HIV envelope protein that has specific mutations introduced to increase the stability of the protein complex. A stable TF4.1-expressing Chinese Hamster Ovary (CHO) pool has been generated and controlled upstream production process has been developed. Described herein is the downstream purification process developed to produce TF4.1 for use as a drug substance.

The downstream process contains three chromatography, two tangential flow filtration, two dedicated viral inactivation/clearance and one bulk filtration operations and typically occurs over a course of 3-6 days. The downstream process starts with the acceptance of ambient clarified harvest from the CHO cell line, followed by TFF consisting of 5-7× concentration then diafiltration into Capture Load buffer. Next, Viral Inactivation (VI) was executed by spiking to a final concentration of 0.5% w/w Triton X-100 and incubating 30 minutes to two hours. The VI Pool was then loaded on the Tosoh NH2-750 Capture Column. The NH2-750 Eluate was directly loaded onto the CHT Intermediate column. Subsequently, the CHT Eluate was nanofiltered. The Nanofiltrate was then diluted to a final concentration of 0.6M ammonium sulfate and passed through a Capto Phenyl chromatography column in flow through mode. Phenyl Flowthrough was concentrated to a target concentration of 1.5 g/L by UV A280 and buffer exchanged into final formulation buffer. The UFDF Retentate was diluted with final formulation buffer to final concentration at 1.3 g/L. The UFDF Retentate was then 0.2 μm filtered, resulting in Bulk Drug Substance that was then frozen at −80° C. A schematic of the downstream process is presented in FIGS. 21A-B.

The first step in the downstream process was tangential flow filtration (ultrafiltration and diafiltration) to prepare clarified harvest for capture load and to control processing volume. With 300 kD nominal MW cutoff, the tangential flow filtration was also a critical purification step used to remove small host cell proteins. The TFF filter was assembled, flushed with purified water and flushed with 20 mM HEPES, 250 mM NaCl, pH 7.2 (DF Buffer). The clarified harvest was then concentrated 5-7×, followed by diafiltration with 5 diavolumes of DF buffer. The product was recirculated and recovered, then the membrane was flushed with DF buffer to increase the step yield. In this step, clarified harvest was concentrated and purified. The TFF retentate was subjected to the next step in the process.

Viral inactivation: The second step in the downstream process, was viral inactivation with Triton X-100. Triton X-100 inactivates enveloped virus by disrupting the viral lipid envelope (Conley et al, 2016). 10% v/v Triton X-100 is added to a final concentration of 0.5% w/w Triton X-100 (1 part 10% Triton X-100, 19 parts Clarified Harvest). The TFF Retentate was mixed and then held at room temperature for at least ½ hours, with or without mixing.

The initial chromatography step (capture) used Toyopearl NH2-750 resin to bind and elute the target molecule into a single bulk fraction. This resin is composed of polymethacrylate beads that have been functionalized with primary amine (NH2) strong anion exchange groups. This step removes host cell contaminants. Loading of Toyopearl NH₂-750 was scaled to load up to 30 L of clarified harvest per liter of resin, based upon historical upstream titers. The packed Toyopearl NH2-750 column was sanitized, equilibrated, loaded, washed, and product fraction is eluted and collected. The column was then stripped, sanitized, and stored.

The second chromatography step (intermediate) used ceramic hydroxyapatite resin to bind and elute the target molecule into a single bulk fraction. This resin has calcium affinity interaction and cation exchange interaction mechanisms of action. This step removed host cell contaminants and product related contaminants. The entire Toyopearl NH2-750 Eluate was loaded, without manipulation, onto the column. The CHT column was charged, equilibrated, loaded, washed, and product fraction was eluted. The column was then stripped, sanitized, and stored. The CHT column can only be operated in downflow.

Viral reduction: The next step in the downstream process was nanofiltration with the Viresolve Pro (Vpro) nanofilter. The nanofilter has a nominal exclusion limit of 20 nm and virus removal was achieved via size partitioning. The nanofilter was protected by placing the Viresolve Shield H guard filter upstream of the Vpro. The CHT Eluate was nanofiltered and a buffer flush was performed, the combined filtered CHT Eluate and flush were the nanofiltrate.

The Capto Phenyl polishing step was operated in flow through mode and removed host cell contaminants and product contaminants. This resin has a phenyl ligand hydrophobic mechanism of action. The nanofiltrate was diluted and loaded onto the column. Prior to load, nanofiltrate was spiked to final concentration 0.6M ammonium sulfate with 20 mM HEPES, 1.2M ammonium sulfate, pH 7.2. The Capto Phenyl column was sanitized, equilibrated, loaded and washed. The load flow through and initial wash was combined into a single product fraction. The column was then stripped, sanitized, and stored.

The next step of the TF4.1 purification process was a UFDF step to concentrate and buffer exchange product into the final formulation buffer. The UFDF filter was assembled, flushed with purified water and flushed with 20 mM Tris, 100 mM NaCl, pH 7.5 (formulation buffer). The product was then concentrated to a target of 1.5 g/L, followed by diafiltration with 7 diavolumes of formulation buffer. The product was recirculated and recovered, then the membrane was flushed with formulation buffer to increase the step yield. The UFDF Retentate and calculated UFDF Flush were pooled and mixed. An absorbance, concentration assay was utilized on the combined UFDF Retentate and UFDF Flush to calculate the appropriate dilution volume to hit the drug substance concentration specification. All components of the final formulation buffer are multi-compendial.

The final step in the downstream process was a 0.2 μm filtration with polyethersulfone (PES) Sartopore 2 and then aseptically bulk filled into the final container suitable for storage.

Scale Demonstration

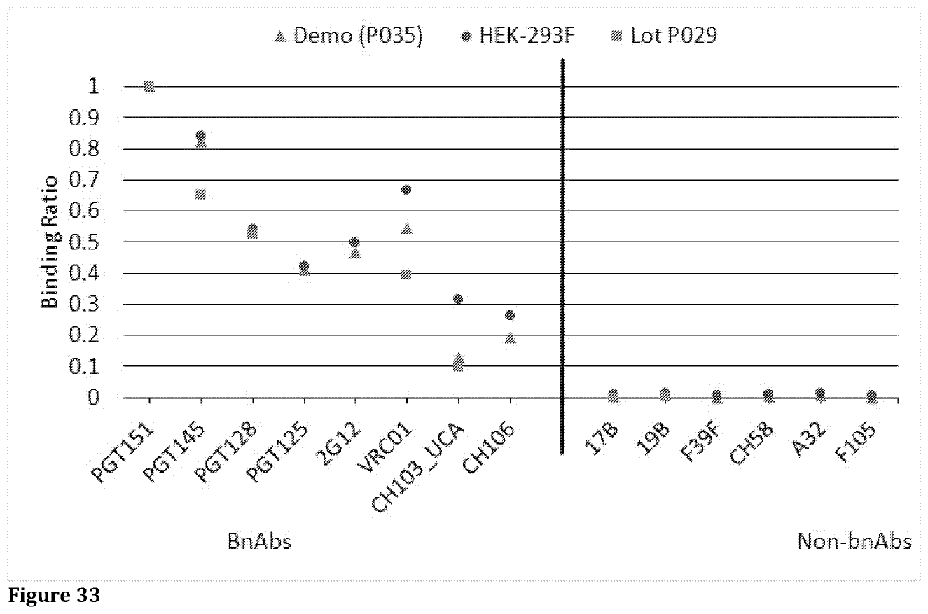

The trimer non-affinity process was demonstrated at multiple scales using commercially available bioprocess resins and filters/membranes via execution at the pilot scale (˜50 L cell culture) and under cGMP manufacturing conditions at clinical production scale (2 runs at ˜200 L cell culture). The productivity and product quality are comparable across scales, as shown in Table 14 and FIG. 33.

TABLE 14
Data demonstrating scalability of the methods for example at
50 L and 200 L bioreactor scale

| | | 200 L Scale | 200 L Scale |
|---|---|---|---|
| Method | 50 L Scale | Run 1 | Run 2 |
| Productivity (mg | 27 | 17 | 18 |
| Drug Substance/L | | | |
| clarified harvest) | | | |
| Purification) | 30 | 21 | 23 |
| Yield (% | | | |
| pH | 7.2 | 7.6 | 7.4 |
| SE-UPLC (%) | 99.7 | 99.4 | 99.3 |
| Absorption at 280 | 1.3 | 1.4 | 1.3 |
| nm (mg/mL) | | | |
| Surface Plasmon | PGT145: | PGT145: | PGT145: |
| Resonance (SPR) | K_{D} = 11.0 nM | K_{D} = 11.3 nM | K_{D} = 11.7 nM |
| Antibody binding | PGT151: | PGT151: | PGT151: |
| | K_{D} = 17.5 nM | K_{D} = 16.0 nM | K_{D} = 13.1 nM |
| Residual HCP | 0.004 μg/mg | BLQ (<1.1 | 3.58 ng/mg |
| ELISA | | ng/mg) | |
| Host cell DNA | Not tested | 4.48 E−01 | <5.00E−01 |
| | | pg/mg | pg/mg |
| Endotoxin | Not tested | <0.04 EU/mg | <0.04 EU/mg |

See also FIG. 33 showing antigenicity of TF 4.1 trimer purified from 50 L CHO cell culture.

### Example 6: Viral Clearance Characterization of the Product Produced in Example 5

Biological products derived from cell lines carry risk of viral contamination, and Chinese hamster ovary cell lines are known to contain endogenous retrovirus like particles (RVLPs). ICH Q5A guidelines (Viral Safety Evaluation of Biotechnology Products Derived from Cell Lines of Human or Animal Origin) require that virus clearance steps must be validated before a Phase I product may be administered to humans. Viral clearance should be demonstrated by at least two orthogonal steps, when possible. Regulatory authorities do not give firm guidelines on minimum viral reduction and an assessment should be made on a product specific basis, however in general the biopharmaceutical industry typically strives for at least 4-6 logs of excess clearance of a model virus.

The trimer non-affinity purification process described in FIG. 21B and Example 5 is comprised of eight unit operations. Five downstream steps were assessed for viral clearance: viral inactivation by Triton X-100, virus removal by three unique chromatography steps (AEX-Toyopearl NH2-750, mixed mode-Ceramic hydroxyapatite, and HIC-Capto Phenyl) and virus removal via Viresolve Pro nanofiltration. Triton X-100 viral inactivation can disrupt the lipid membrane of enveloped viruses, thus disrupting the ability of the virus to infect cells. Chromatography provides a specific binding mechanism in which the virus and product are separated by physicochemical properties (charge, hydrophobicity, etc.). Viral filtration is a partitioning method that works by size exclusion.

The two viruses selected for the viral clearance validation study, xenotropic murine leukemia virus and mouse minute virus, were chosen to represent known Chinese hamster ovary cell endogenous retrovirus like particle contaminants and to incorporate a range of virus properties. Viruses chosen for this study are detailed in Table 15 below.

TABLE 15
Model Virus Selection

| | | | | | Physico- | |
|---|---|---|---|---|---|---|
| | | | | | Chemical | |
| | | | | Size | Inactivation | Representative |
| Model | Family | Genome | Enveloped | (nm) | Resistance | Virus |
| XMuLV | | | | | | |
| Xenotropic | Retro | ss-RNA | Yes | 70-100 | Low | Model for CHO |
| Murine | | | | | | C-type retrovirus |
| Leukemia Virus | | | | | | |
| MMV | | | | | | |
| Mouse Minute | Parvo | ss-DNA | No | 18-24 | High | Physico-chemical |
| Virus | | | | | | resistant virus |

Total virus reduction demonstrated by the five identified operations provides adequate clearance, with orthogonal mechanisms of reduction and significant safety factor, of both XMuLV and MMV. The additive log 10 reduction value (LRV) of XMuLV is 19.40. Additive clearance of MMV is 12.18 logs, which provides a significant safety factor for adventitious viruses.

The log reduction values for each step and the overall process are shown in Table 16.

TABLE 16
Log10 Reduction Values for Purification Process

| | Run # | Log | |
|---|---|---|---|
| | (Each | Reduction | |
| | step run in | Value | |
| Step | duplicate) | XMuLV | MMV |
| Triton X-100 Inactivation | 1 | ≥1.49 | NA |
| | 2 | ≥1.67 | NA |
| Toyopearl NH2-750 | 1 | ≥5.86 | ≥5.66 |
| | 2 | ≥5.88 | ≥5.62 |
| CHT Intermediate | 1 | 3.49 | NR |
| | 2 | 4.10 | NR |
| Capto Phenyl | 1 | 3.05 | 1.31 |
| | 2 | 3.24 | 1.75 |
| Viresolve Pro | 1 | ≥5.51 | ≥5.25 |
| Nanofiltration | 2 | ≥5.61 | ≥5.26 |
| Overall Process* | | 19.40 | 12.18 |

NR: No Reduction,
NA: Not Applicable
*Calculated using lowest log reduction factor for each step

What is claimed is:

1. A method of purifying a recombinant viral envelope protein, the method comprising:
a. step (a) contacting an anion exchange (AEX) chromatography resin with a fraction (1) comprising recombinant viral envelope protein,
b. step (b) eluting a fraction (2) from the resin of step (a),
c. step (c) contacting a mixed-mode chromatography resin with the fraction (2) from step (b), and
d. step (d) eluting a fraction (3) from the resin of step (c),
i. wherein fraction (3) has fewer product-related impurities compared to fraction (1) or fraction (2).

2. The method of claim 1, wherein the method further comprises:
e. step (e) contacting a hydrophobic interaction chromatography (HIC) resin with fraction (3) from step (d), and
f. step (f) collecting unbound flow through as fraction (4),
i. wherein fraction (4) has fewer product-related impurities compared to fraction (1), fraction (2), or fraction (3).

3. A method of purifying a recombinant viral envelope protein, the method comprising:
a. step (a) contacting an anion exchange (AEX) chromatography resin with a fraction (1) comprising a recombinant viral envelope protein,
b. step (b) eluting a fraction (2) from the resin of step (a),
c. step (c) contacting a HIC resin with fraction (2) from step (b), and
d. step (d) collecting flow through from step (c) as fraction (3),
i. wherein fraction (3) has fewer product-related impurities compared to fraction (1) or fraction (2).

4. A method of purifying a recombinant viral envelope protein, the method comprising:
a. step (a) contacting a mixed-mode chromatography resin with a fraction (1) comprising a recombinant viral envelope protein,
b. step (b) eluting a fraction (2) from the resin of step (a),
c. step (c) contacting a HIC resin with fraction (2) from step (b), and
d. step (d) eluting a fraction (3) from the resin of step (c),
i. wherein fraction (3) has fewer product-related impurities compared to fraction (1) or fraction (2).

5. The method of any one of claims 1-4, further comprising a viral reduction step.

6. A method of purifying a recombinant nanoparticle comprising a recombinant viral envelope protein, the method comprising:
a. step (a) contacting a multi-mode resin, with a fraction (1) comprising recombinant nanoparticle,
b. step (b) collecting a flow through from step (a) as fraction (2),
c. step (c) contacting an anion exchange (AEX) chromatography resin, with the flow through fraction (2), and
d. step (d) eluting a fraction (3) from the resin of step (c),
i. wherein fraction (3) has fewer product-related impurities compared to fraction (1) or fraction (2).

7. The method of claim 6 further comprising:
e. step (e) contacting a HIC resin with fraction (3) from step (d) under conditions suitable for flow through operation or binding to the HIC resin, and
f. step (f) collecting from the resin of step (e) unbound flow through as fraction (4) under suitable conditions or eluting a fraction (4) under suitable conditions,
i. wherein fraction (4) has fewer product-related impurities compared to fraction (1), fraction (2), or fraction (3).

8. The method of claim 6 further comprising:
e. step (e) contacting a mixed mode resin with fraction (3) from step (d), and
f. step (f) eluting from the resin of step (e) a fraction (4),
i. wherein fraction (4) has fewer product related impurities compared to fraction (1), fraction (2), or fraction (3).

9. The method of any one of the preceding claims wherein the recombinant viral envelope protein is an HIV-1 envelope protein, wherein the HIV-1 envelope protein comprise a gp140 sequence designed to form a stable trimer.

10. The method of any of the preceding claims wherein the recombinant viral envelope protein is CH505 T/F trimer.

11. The method of any one of claim 9, wherein the AEX resin is contacted with fraction (1) in 250 mM salt buffer.

12. The method of any one of claim 9, wherein fraction (2) is eluted from the AEX resin is in 600 mM salt buffer.

13. The method of claim 9, wherein the mixed mode resin is contacted with fraction (2) in 600 mM salt buffer.

14. The method of claim 9, wherein fraction (3) is eluted from the mixed mode resin is in 30 mM phosphate buffer.

15. The method of claim 9, wherein the HIC resin is contacted with fraction (3) in 600 mM ammonium sulfate.

16. The method of claim 9, wherein the flow through fraction (4) is collected in 600 mM ammonium sulfate.

17. The method of any of the preceding claims, wherein all steps are conducted at pH 7.0-7.4.

18. The method of any of the preceding claims, wherein fraction (3) or fraction (4) comprises a well-folded trimer.

19. The method of any of the preceding claims, wherein faction (3) or fraction (4) comprises a nanoparticle comprising well-folded trimers.

20. The method of any one of claims 1-15, wherein the method further comprises at least one viral reduction step.

21. The method of any one of the preceding claims, wherein fraction (1) is a harvest pool from a bioreactor culture of 20 L to 20,000 L.

22. The method of any one of the preceding claims where the fraction (1) is a harvest pool subjected to a Tangential Flow Filtration (TFF) step.


SEQUENCE LISTING (48 sequences)

SEQ ID NO: 1 - DNA, 2007 bp, Artificial Sequence
  Description of Artificial Sequence Synthetic polynucleotide
gtcgacaagcttgccaccatgagggtccggggaatccagcgcaactgccagcacctctggaggtggggcacgctgatcctggggatgctgatgatctgcagcgcggctgagaacctgtgggtgacagtgtactacggcgtgcctgtgtggaaggaggccaacaccaccctgttctgcgcctcggacgccaaggcctacgacacggaggtccacaacgtgtgggctacccacgcctgcgtgcccaccgaccccaatcctcaggagatcgtcctggagaacgtgaccgagaacttcaacatgtggaagaacaacatggtggagcagatgcacgaggacatcatcagcctgtgggaccagagcctgaagccctgcgtgaagctgacccccctgtgcgtgaccctgaactgcacgaacgtgaacgtgaccaacaccacgaacaacacggaggagaagggggagatcaagaactgcagcttcaacatcaccaccgagatccgggacaagaagcagaaggtgtacgccctgttctaccggctggacgtcgtgccgatcgacgacaacaacaacaactccagcaactacaggctgatcaactgcaacaccagcgcgatcacccaggcctgccctaaggtgtcgttcgagcccatccccatccactactgcgcgcctgccggcttcgccatcctgaagtgcaacgacaagaagttcaacggcaccggcccctgcaagaacgtcagcaccgtccagtgcacccacggcatcaagcctgtggtgtccacccagctgctcctgaacggcagcctggccgaggaggagatcatcatcaggagcgagaacatcaccaacaacgccaagacgatcatcgtgcagctgaacgagtcggtggagatcaactgcacccggcccaacaacaacacgcggaagagcatccggatcggccctggacaggcgttctacgccacgggcgacatcatcggcgacatcaggcaggcccactgcaacatctcggggacgaagtggaacaagaccctgcagcaggtcgcgaagaagctgagggagcacttcaacaacaagaccatcatcttcaagccgagcagcggcggagacctggagatcaccacgcactcgttcaactgccggggcgagttcttctactgtaacacgtcgggcctgttcaacagcacctggatcggcaacggcacgaagaacaacaacaacactaacgacaccatcaccctgccctgccggatcaagcagatcatcaacatgtggcagggcgtgggccaggctatgtacgcccctcccatcgagggcaagatcacgtgcaagagcaacatcaccggcctgctgctgaccagggacggcgggaacaacaacacgaacgagaccgagatcttcagacctggcggcggagacatgagagacaactggcggagcgagctgtacaagtacaaggtcgtgaagatcgagcccctgggcgtcgcacccaccaagtgcaaggagagggtggtgggcaggcgacgccgtaggcgggcggtcggcatcggcgccgtgttcctgggcttcctgggagcagccggcagcaccatgggagccgcctcgatcaccctgaccgtgcaggcgaggcagctgctgtccggcatcgtgcagcagcagtcgaacctgctgagggcccccgaggcccagcagcacctgctccagctgaccgtgtggggcatcaagcagctccaggccagggtgctggccgtcgagcgctacctgaaggaccagcagctgctcggcatctggggctgcagcggcaagctgatctgctgcaccaccgtgccctggaacagcagctggagcaacaagagccaggacgagatctgggacaacatgacctggatggagtgggagcgggagatcaacaactacaccgacatcatctacagcctgatcgaggagagccagaaccagcaggagaagaacgagcaggagctgctggcgctggactgatctagaggatcc

SEQ ID NO: 2 - DNA, 2007 bp, Artificial Sequence
  Description of Artificial Sequence Synthetic polynucleotide
gtcgacaagcttgccaccatgagggtccggggaatccagcgcaactgccagcacctctggaggtggggcacgctgatcctggggatgctgatgatctgcagcgcggctgagaacctgtgggtgacagtgtactacggcgtgcctgtgtggaaggaggccaacaccaccctgttctgcgcctcggacgccaaggcctacgacacggaggtccacaacgtgtgggctacccacgcctgcgtgcccaccgaccccaatcctcaggagatcgtcctggagaacgtgaccgagaacttcaacatgtggaagaacaacatggtggagcagatgcacgaggacatcatcagcctgtgggaccagagcctgaagccctgcgtgaagctgacccccctgtgcgtgaccctgaactgcacgaacgtgaacgtgaccaacaccacgaacaacacggaggagaagggggagatcaagaactgcagcttcaacatcaccaccgagatccgggacaagaagcagaaggtgtacgccctgttctaccggctggacgtcgtgccgatcgacgacaacaacaacaactccagcaactacaggctgatcaactgcaacaccagcgcgtgcacccaggcctgccctaaggtgtcgttcgagcccatccccatccactactgcgcgcctgccggcttcgccatcctgaagtgcaacgacaagaagttcaacggcaccggcccctgcaagaacgtcagcaccgtccagtgcacccacggcatcaagcctgtggtgtccacccagctgctcctgaacggcagcctggccgaggaggagatcatcatcaggagcgagaacatcaccaacaacgccaagacgatcatcgtgcagctgaacgagtcggtggagatcaactgcacccggcccaacaacaacacgcggaagagcatccggatcggccctggacaggcgttctacgccacgggcgacatcatcggcgacatcaggcaggcccactgcaacatctcggggacgaagtggaacaagaccctgcagcaggtcgcgaagaagctgagggagcacttcaacaacaagaccatcatcttcaagccgagcagcggcggagacctggagatcaccacgcactcgttcaactgccggggcgagttcttctactgtaacacgtcgggcctgttcaacagcacctggatcggcaacggcacgaagaacaacaacaacactaacgacaccatcaccctgccctgccggatcaagcagatcatcaacatgtggcagggcgtgggccagtgtatgtacgcccctcccatcgagggcaagatcacgtgcaagagcaacatcaccggcctgctgctgaccagggacggcgggaacaacaacacgaacgagaccgagatcttcagacctggcggcggagacatgagagacaactggcggagcgagctgtacaagtacaaggtcgtgaagatcgagcccctgggcgtcgcacccaccaagtgcaaggagagggtggtgggcaggcgacgccgtaggcgggcggtcggcatcggcgccgtgttcctgggcttcctgggagcagccggcagcaccatgggagccgcctcgatcaccctgaccgtgcaggcgaggcagctgctgtccggcatcgtgcagcagcagtcgaacctgctgagggcccccgaggcccagcagcacctgctccagctgaccgtgtggggcatcaagcagctccaggccagggtgctggccgtcgagcgctacctgaaggaccagcagctgctcggcatctggggctgcagcggcaagctgatctgctgcaccaccgtgccctggaacagcagctggagcaacaagagccaggacgagatctgggacaacatgacctggatggagtgggagcgggagatcaacaactacaccgacatcatctacagcctgatcgaggagagccagaaccagcaggagaagaacgagcaggagctgctggcgctggactgatctagaggatcc

SEQ ID NO: 3 - DNA, 2007 bp, Artificial Sequence
  Description of Artificial Sequence Synthetic polynucleotide
gtcgacaagcttgccaccatgagggtccggggaatccagcgcaactgccagcacctctggaggtggggcacgctgatcctggggatgctgatgatctgcagcgcggctgagaacctgtgggtgacagtgtactacggcgtgcctgtgtggaaggaggccaacaccaccctgttctgcgcctcggacgccaaggcctacgacacggaggtccacaacgtgtgggctacccacgcctgcgtgcccaccgaccccaatcctcaggagatcgtcctggagaacgtgaccgagaacttcaacatgtggaagaacaacatggtggagcagatgcacgaggacatcatcagcctgtgggaccagagcctgaagccctgcgtgaagctgacccccctgtgcgtgaccctgaactgcacgaacgtgaacgtgaccaacaccacgaacaacacggaggagaagggggagatcaagaactgcagcttcaacatcaccaccgagatccgggacaagaagcagaaggtgtacgccctgttctaccggctggacgtcgtgccgatcgacgacaacaacaacaactccagcaactacaggctgatcaactgcaacaccagcgcgatcacccaggcctgccctaaggtgtcgttcgagcccatccccatccactactgcgcgcctgccggcttcgccatcctgaagtgcaacgacaagaagttcaacggcaccggcccctgcaagaacgtcagcaccgtccagtgcacccacggcatcaagcctgtggtgtccacccagctgctcctgaacggcagcctggccgaggaggagatcatcatcaggagcgagaacatcaccaacaacgccaagacgatcatcgtgcagctgaacgagtcggtggagatcaactgcacccggcccaacaacaacacgcggaagagcatccggatcggccctggacaggcgttctacgccacgggcgacatcatcggcgacatcaggcaggcccactgcaacatctcggggacgaagtggaacaagaccctgcagcaggtcgcgaagaagctgagggagcacttcaacaacaagaccatcatcttcaagccgagcagcggcggagacctggagatcaccacgcactcgttcaactgccggggcgagttcttctactgtaacacgtcgggcctgttcaacagcacctggatcggcaacggcacgaagaacaacaacaacactaacgacaccatcaccctgccctgccggatcaagcagatcatcaacatgtggcagggcgtgggccaggctatgtacgcccctcccatcgagggcaagatcacgtgcaagagcaacatcaccggcctgctgctgaccagggacggcgggaacaacaacacgaacgagaccgagatcttcagacctggcggcggagacatgagagacaactggcggagcgagctgtacaagtacaaggtcgtgaagatcgagcccctgggcgtcgcacccaccaagtgcaaggagagggtggtgggcaggcgacgccgtaggcgggcggtcggcatcggcgccgtgttcctgggcttcctgggagcagccggcagcaccatgggagccgcctcgatgaccctgaccgtgcaggcgaggcagctgctgtccggcatcgtgcagcagcagtcgaacctgctgagggcccccgaggcccagcagcacctgctccagctgaccgtgtggggcatcaagcagctccaggccagggtgctggccgtcgagcgctacctgaaggaccagcagctgctcggcatctggggctgcagcggcaagctgatctgctgcaccaccgtgccctggaacagcagctggagcaacaagagccaggacgagatctgggacaacatgacctggatggagtgggagcgggagatcaacaactacaccgacatcatctacagcctgatcgaggagagccagaaccagcaggagaagaacgagcaggagctgctggcgctggactgatctagaggatcc

SEQ ID NO: 4 - DNA, 2007 bp, Artificial Sequence
  Description of Artificial Sequence Synthetic polynucleotide
gtcgacaagcttgccaccatgagggtccggggaatccagcgcaactgccagcacctctggaggtggggcacgctgatcctggggatgctgatgatctgcagcgcggctgagaacctgtgggtgacagtgtactacggcgtgcctgtgtggaaggaggccaacaccaccctgttctgcgcctcggacgccaaggcctacgacacgaaggtccacaacgtgtgggctacccacgcctgcgtgcccaccgaccccaatcctcaggagatcgtcctggagaacgtgaccgagaacttcaacatgtggaagaacaacatggtggagcagatgcacgaggacatcatcagcctgtgggaccagagcctgaagccctgcgtgaagctgacccccctgtgcgtgaccctgaactgcacgaacgtgaacgtgaccaacaccacgaacaacacggaggagaagggggagatcaagaactgcagcttcaacatcaccaccgagatccgggacaagaagcagaaggtgtacgccctgttctaccggctggacgtcgtgccgatcgacgacaacaacaacaactccagcaactacaggctgatcaactgcaacaccagcgcgatcacccaggcctgccctaaggtgtcgttcgagcccatccccatccactactgcgcgcctgccggcttcgccatcctgaagtgcaacgacaagaagttcaacggcaccggcccctgcaagaacgtcagcaccgtccagtgcacccacggcatcaagcctgtggtgtccacccagctgctcctgaacggcagcctggccgaggaggagatcatcatcaggagcgagaacatcaccaacaacgccaagacgatcatcgtgcagctgaacgagtcggtggagatcaactgcacccggcccaacaacaacacgcggaagagcatccggatcggccctggacagtggttctacgccacgggcgacatcatcggcgacatcaggcaggcccactgcaacatctcggggacgaagtggaacaagaccctgcagcaggtcgcgaagaagctgagggagcacttcaacaacaagaccatcatcttcaagccgagcagcggcggagacctggagatcaccacgcactcgttcaactgccggggcgagttcttctactgtaacacgtcgggcctgttcaacagcacctggatcggcaacggcacgaagaacaacaacaacactaacgacaccatcaccctgccctgccggatcaagcagatcatcaacatgtggcagggcgtgggccaggctatgtacgcccctcccatcgagggcaagatcacgtgcaagagcaacatcaccggcctgctgctgaccagggacggcgggaacaacaacacgaacgagaccgagatcttcagacctggcggcggagacatgagagacaactggcggagcgagctgtacaagtacaaggtcgtgaagatcgagcccctgggcgtcgcacccaccaagtgcaaggagagggtggtgggcaggcgacgccgtaggcgggcggtcggcatcggcgccgtgttcctgggcttcctgggagcagccggcagcaccatgggagccgcctcgatgaccctgaccgtgcaggcgaggcagctgctgtccggcatcgtgcagcagcagtcgaacctgctgagggcccccgaggcccagcagcacctgctccagctgaccgtgtggggcatcaagcagctccaggccagggtgctggccgtcgagcgctacctgaaggaccagcagctgctcggcatctggggctgcagcggcaagctgatctgctgcaccaccgtgccctggaacagcagctggagcaacaagagccaggacgagatctgggacaacatgacctggatggagtgggagcgggagatcaacaactacaccgacatcatctacagcctgatcgaggagagccagaaccagcaggagaagaacgagcaggagctgctggcgctggactgatctagaggatcc

SEQ ID NO: 5 - DNA, 2007 bp, Artificial Sequence
  Description of Artificial Sequence Synthetic polynucleotide
gtcgacaagcttgccaccatgagggtccggggaatccagcgcaactgccagcacctctggaggtggggcacgctgatcctggggatgctgatgatctgcagcgcggctgagaacctgtgggtgacagtgtactacggcgtgcctgtgtggaaggaggccaacaccaccctgttctgcgcctcggacgccaaggcctacgacacggaggtcagaaacgtgtgggctacccacgcctgcgtgcccaccgaccccaatcctcaggagatcgtcctggagaacgtgaccgagaacttcaacatgtggaagaacaacatggtggagcagatgcacgaggacatcatcagcctgtgggaccagagcctgaagccctgcgtgaagctgacccccctgtgcgtgaccctgaactgcacgaacgtgaacgtgaccaacaccacgaacaacacggaggagaagggggagatcaagaactgcagcttcaacatcaccaccgagatccgggacaagaagcagaaggtgtacgccctgttctaccggctggacgtcgtgccgatcgacgacaacaacaacaactccagcaactacaggctgatcaactgcaacaccagcgcgatcacccaggcctgccctaaggtgtcgttcgagcccatccccatccactactgcgcgcctgccggcttcgccatcctgaagtgcaacgacaagaagttcaacggcaccggcccctgcaagaacgtcagcaccgtccagtgcacccacggcatcaagcctgtggtgtccacccagctgctcctgaacggcagcctggccgaggaggagatcatcatcaggagcgagaacatcaccaacaacgccaagacgatcatcgtgcagctgaacgagtcggtggagatcaactgcacccggcccaacaacaacacgcggaagagcatccggatcggccctggacagtggttctacgccacgggcgacatcatcggcgacatcaggcaggcccactgcaacatctcggggacgaagtggaacaagaccctgcagcaggtcgcgaagaagctgagggagcacttcaacaacaagaccatcatcttcaagccgagcagcggcggagacctggagatcaccacgcactcgttcaactgccggggcgagttcttctactgtaacacgtcgggcctgttcaacagcacctggatcggcaacggcacgaagaacaacaacaacactaacgacaccatcaccctgccctgccggatcaagcagatcatcaacatgtggcagggcgtgggccaggctatgtacgcccctcccatcgagggcaagatcacgtgcaagagcaacatcaccggcctgctgctgaccagggacggcgggaacaacaacacgaacgagaccgagatcttcagacctggcggcggagacatgagagacaactggcggagcgagctgtacaagtacaaggtcgtgaagatcgagcccctgggcgtcgcacccaccaagtgcaaggagagggtggtgggcaggcgacgccgtaggcgggcggtcggcatcggcgccgtgttcctgggcttcctgggagcagccggcagcaccatgggagccgcctcgatgaccctgaccgtgcaggcgaggcagctgctgtccggcatcgtgcagcagcagtcgaacctgctgagggcccccgaggcccagcagcacctgctccagctgaccgtgtggggcatcaagcagctccaggccagggtgctggccgtcgagcgctacctgaaggaccagcagctgctcggcatctggggctgcagcggcaagctgatctgctgcaccaccgtgccctggaacagcagctggagcaacaagagccaggacgagatctgggacaacatgacctggatggagtgggagcgggagatcaacaactacaccgacatcatctacagcctgatcgaggagagccagaaccagcaggagaagaacgagcaggagctgctggcgctggactgatctagaggatcc

SEQ ID NO: 6 - DNA, 2025 bp, Artificial Sequence
  Description of Artificial Sequence Synthetic polynucleotide
gtcgacgccaccatgggctccctgcagcccctggccaccctgtacctgctgggcatgctggtggcctccgtgctggccgccgagaacctgtgggtgaccgtgtactacggcgtgcccgtgtggaaggaggccaacaccaccctgttctgcgcctccgacgccaaggcctacgacaccgaggtgcacaacgtgtgggccacccacgcctgcgtgcccaccgaccccaacccccaggagatcgtgctggagaacgtgaccgagaacttcaacatgtggaagaacaacatggtggagcagatgcacgaggacatcatctccctgtgggaccagtccctgaagccctgcgtgaagctgacccccctgtgcgtgaccctgaactgcaccaacgtgaacgtgaccaacaccaccaacaacaccgaggagaagggcgagatcaagaactgctccttcaacatcaccaccgagatccgcgacaagaagcagaaggtgtacgccctgttctaccgcctggacgtggtgcccatcgacgacaacaacaacaactcctccaactaccgcctgatcaactgcaacacctccgccatcacccaggcctgccccaaggtgtccttcgagcccatccccatccactactgcgcccccgccggcttcgccatcctgaagtgcaacgacaagaagttcaacggcaccggcccctgcaagaacgtgtccaccgtgcagtgcacccacggcatcaagcccgtggtgtccacccagctgctgctgaacggctccctggccgaggaggagatcatcatccgctccgagaacatcaccaacaacgccaagaccatcatcgtgcagctgaacgagtccgtggagatcaactgcacccgccccaacaacaacacccgcaagtccatccgcatcggccccggccaggccttctacgccaccggcgacatcatcggcgacatccgccaggcccactgcaacatctccggcaccaagtggaacaagaccctgcagcaggtggccaagaagctgcgcgagcacttcaacaacaagaccatcatcttcaagccctcctccggcggcgacctggagatcaccacccactccttcaactgccgcggcgagttcttctactgcaacacctccggcctgttcaactccacctggatcggcaacggcaccaagaacaacaacaacaccaacgacaccatcaccctgccctgccgcatcaagcagatcatcaacatgtggcagggcgtgggccaggccatgtacgccccccccatcgagggcaagatcacctgcaagtccaacatcaccggcctgctgctgacccgcgacggcggcaacaacaacaccaacgagaccgagatcttccgccccggcggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggacggctccggcctgaacgacatcttcgaggcccagaagatcgagtggcacgagtagggatcc

SEQ ID NO: 7 - DNA, 2025 bp, Artificial Sequence
  Description of Artificial Sequence Synthetic polynucleotide
gtcgacgccaccatgggctccctgcagcccctggccaccctgtacctgctgggcatgctggtggcctccgtgctggccgccgagaacctgtgggtgaccgtgtactacggcgtgcccgtgtggaaggaggccaacaccaccctgttctgcgcctccgacgccaaggcctacgacaccgaggtgcacaacgtgtgggccacccacgcctgcgtgcccaccgaccccaacccccaggagatcgtgctggagaacgtgaccgagaacttcaacatgtggaagaacaacatggtggagcagatgcacgaggacatcatctccctgtgggaccagtccctgaagccctgcgtgaagctgacccccctgtgcgtgaccctgaactgcaccaacgtgaacgtgaccaacaccaccaacaacaccgaggagaagggcgagatcaagaactgctccttcaacatcaccaccgagatccgcgacaagaagcagaaggtgtacgccctgttctaccgcctggacgtggtgcccatcgacgacaacaacaacaactcctccaactaccgcctgatcaactgcaacacctccgcctgcacccaggcctgccccaaggtgtccttcgagcccatccccatccactactgcgcccccgccggcttcgccatcctgaagtgcaacgacaagaagttcaacggcaccggcccctgcaagaacgtgtccaccgtgcagtgcacccacggcatcaagcccgtggtgtccacccagctgctgctgaacggctccctggccgaggaggagatcatcatccgctccgagaacatcaccaacaacgccaagaccatcatcgtgcagctgaacgagtccgtggagatcaactgcacccgccccaacaacaacacccgcaagtccatccgcatcggccccggccaggccttctacgccaccggcgacatcatcggcgacatccgccaggcccactgcaacatctccggcaccaagtggaacaagaccctgcagcaggtggccaagaagctgcgcgagcacttcaacaacaagaccatcatcttcaagccctcctccggcggcgacctggagatcaccacccactccttcaactgccgcggcgagttcttctactgcaacacctccggcctgttcaactccacctggatcggcaacggcaccaagaacaacaacaacaccaacgacaccatcaccctgccctgccgcatcaagcagatcatcaacatgtggcagggcgtgggccagtgcatgtacgccccccccatcgagggcaagatcacctgcaagtccaacatcaccggcctgctgctgacccgcgacggcggcaacaacaacaccaacgagaccgagatcttccgccccggcggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggacggctccggcctgaacgacatcttcgaggcccagaagatcgagtggcacgagtagggatcc

SEQ ID NO: 8 - DNA, 2025 bp, Artificial Sequence
  Description of Artificial Sequence Synthetic polynucleotide
gtcgacgccaccatgggctccctgcagcccctggccaccctgtacctgctgggcatgctggtggcctccgtgctggccgccgagaacctgtgggtgaccgtgtactacggcgtgcccgtgtggaaggaggccaacaccaccctgttctgcgcctccgacgccaaggcctacgacaccaaggtgcacaacgtgtgggccacccacgcctgcgtgcccaccgaccccaacccccaggagatcgtgctggagaacgtgaccgagaacttcaacatgtggaagaacaacatggtggagcagatgcacgaggacatcatctccctgtgggaccagtccctgaagccctgcgtgaagctgacccccctgtgcgtgaccctgaactgcaccaacgtgaacgtgaccaacaccaccaacaacaccgaggagaagggcgagatcaagaactgctccttcaacatcaccaccgagatccgcgacaagaagcagaaggtgtacgccctgttctaccgcctggacgtggtgcccatcgacgacaacaacaacaactcctccaactaccgcctgatcaactgcaacacctccgccatcacccaggcctgccccaaggtgtccttcgagcccatccccatccactactgcgcccccgccggcttcgccatcctgaagtgcaacgacaagaagttcaacggcaccggcccctgcaagaacgtgtccaccgtgcagtgcacccacggcatcaagcccgtggtgtccacccagctgctgctgaacggctccctggccgaggaggagatcatcatccgctccgagaacatcaccaacaacgccaagaccatcatcgtgcagctgaacgagtccgtggagatcaactgcacccgccccaacaacaacacccgcaagtccatccgcatcggccccggccagtggttctacgccaccggcgacatcatcggcgacatccgccaggcccactgcaacatctccggcaccaagtggaacaagaccctgcagcaggtggccaagaagctgcgcgagcacttcaacaacaagaccatcatcttcaagccctcctccggcggcgacctggagatcaccacccactccttcaactgccgcggcgagttcttctactgcaacacctccggcctgttcaactccacctggatcggcaacggcaccaagaacaacaacaacaccaacgacaccatcaccctgccctgccgcatcaagcagatcatcaacatgtggcagggcgtgggccaggccatgtacgccccccccatcgagggcaagatcacctgcaagtccaacatcaccggcctgctgctgacccgcgacggcggcaacaacaacaccaacgagaccgagatcttccgccccggcggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggacggctccggcctgaacgacatcttcgaggcccagaagatcgagtggcacgagtagggatcc

SEQ ID NO: 9 - DNA, 2025 bp, Artificial Sequence
  Description of Artificial Sequence Synthetic polynucleotide
gtcgacgccaccatgggctccctgcagcccctggccaccctgtacctgctgggcatgctggtggcctccgtgctggccgccgagaacctgtgggtgaccgtgtactacggcgtgcccgtgtggaaggaggccaacaccaccctgttctgcgcctccgacgccaaggcctacgacaccgaggtgcgcaacgtgtgggccacccacgcctgcgtgcccaccgaccccaacccccaggagatcgtgctggagaacgtgaccgagaacttcaacatgtggaagaacaacatggtggagcagatgcacgaggacatcatctccctgtgggaccagtccctgaagccctgcgtgaagctgacccccctgtgcgtgaccctgaactgcaccaacgtgaacgtgaccaacaccaccaacaacaccgaggagaagggcgagatcaagaactgctccttcaacatcaccaccgagatccgcgacaagaagcagaaggtgtacgccctgttctaccgcctggacgtggtgcccatcgacgacaacaacaacaactcctccaactaccgcctgatcaactgcaacacctccgccatcacccaggcctgccccaaggtgtccttcgagcccatccccatccactactgcgcccccgccggcttcgccatcctgaagtgcaacgacaagaagttcaacggcaccggcccctgcaagaacgtgtccaccgtgcagtgcacccacggcatcaagcccgtggtgtccacccagctgctgctgaacggctccctggccgaggaggagatcatcatccgctccgagaacatcaccaacaacgccaagaccatcatcgtgcagctgaacgagtccgtggagatcaactgcacccgccccaacaacaacacccgcaagtccatccgcatcggccccggccagtggttctacgccaccggcgacatcatcggcgacatccgccaggcccactgcaacatctccggcaccaagtggaacaagaccctgcagcaggtggccaagaagctgcgcgagcacttcaacaacaagaccatcatcttcaagccctcctccggcggcgacctggagatcaccacccactccttcaactgccgcggcgagttcttctactgcaacacctccggcctgttcaactccacctggatcggcaacggcaccaagaacaacaacaacaccaacgacaccatcaccctgccctgccgcatcaagcagatcatcaacatgtggcagggcgtgggccaggccatgtacgccccccccatcgagggcaagatcacctgcaagtccaacatcaccggcctgctgctgacccgcgacggcggcaacaacaacaccaacgagaccgagatcttccgccccggcggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggacggctccggcctgaacgacatcttcgaggcccagaagatcgagtggcacgagtagggatcc

SEQ ID NO: 10 - DNA, 2499 bp, Artificial Sequence
  Description of Artificial Sequence Synthetic polynucleotide
gtcaccgtcgtcgacgctagcaccatgggctcgctccagccgctcgcgacgctgtacctcctgggcatgctcgtggcgtccgtgctggcggccgagaacctgtgggtgacggtgtactacggcgtgcccgtgtggaaggaggccaacaccacgctgttctgcgccagcgacgccaaggcctacgacaccgaggtgcacaacgtgtgggcgacccacgcctgcgtgccgacggaccccaacccccaggagatcgtgctggagaacgtgaccgagaacttcaacatgtggaagaacaacatggtggagcagatgcacgaggacatcatctcgctgtgggaccagtccctgaagccgtgcgtgaagctgacgcccctgtgcgtgaccctggactgcaccaacgtgaaggtgacgtccaccacgtccaacacggaggagaagggggagatcaagaactgctccttcaacatcaccaccgagatccgcgacaagaagcagaaggtgtacgcgctgttctaccggctggacgtggtgccgatcgacgacaacaacaacaactccagcaactaccgcctgatcaactgcaacaccagcgcctgcacccaggcctgcccgaaggtgtccttcgagcccatccccatccactactgcgcgccggccggcttcgccatcctgaagtgcaacgacaagaagttcaacggcaccggcccctgcaagaacgtgtccaccgtgcagtgcacccacgggatcaagcccgtggtgtccacgcagctgctgctgaacggctccctggccgaggaggagatcatcatccgctccgagaacatcacgaacaacgccaagaccatcatcgtgcagctgaacgagtccgtggagatcaactgcaccaggcccaacaacaacacccgcaagtccatccggatcggccctggccaggcgttctacgccaccggcgacatcatcggcgacatccgccaggcgcactgcaacatctcgggcacgaagtggaacaagaccctgcagcaggtggcgaagaagctgcgcgagcacttcaacaacaagaccatcatcttcaagcccagctccggcggcgacctggagatcacgacccactccttcaactgccgcggcgagttcttctactgcaacacctccggcctgttcaactcgacgtggatcgggaacggcacgaagaacaacaacaacaccaacgacaccatcaccctgccctgccgcatcaagcagatcatcaacatgtggcagggcgtgggccagtgcatgtacgcgccgcccatcgagggcaagatcacctgcaagtccaacatcaccggcctgctcctgacgcgcgacggcggcaacaacaacaccaacgagaccgagatcttcaggccgggcggcggcgacatgcgcgacaactggcgctcggagctgtacaagtacaaggtggtgaagatcgagcccctgggcgtggcgccgacgcgctgcaagagacgcgtggtgggccgcagacgaaggagacgggccgtgggcatcggcgcggtgttcctgggcttcctgggagcagctggttcgacgatgggcgcagcttccatgaccctgacagtgcaggcacgcaacctgctctccggcatcgtccagcagcagtcgaacctgcttcgagcccccgaggcgcagcagcacctcctcaagctgaccgtgtggggcatcaagcagctgcaggcacgcgtgctagccgtggagcgctacctccgcgaccagcagctgctcggaatctggggctgctcgggcaagctgatctgctgcaccaacgtgccgtggaacagctcctggtccaaccgcaacctctcggagatctgggacaacatgacctggctccagtgggacaaggagatctcgaactacacccagatcatctacggcctgctggaggagtcccagaaccagcaggagaagaacgagcaggacctgctggccctggacggcggaggatctggcgacattatcaagctgctgaacgagcaagtgaacaaagagatgaacagctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgataatctagaggatcc

SEQ ID NO: 11 - DNA, 1986 bp, Artificial Sequence
  Description of Artificial Sequence Synthetic polynucleotide
gtcgacgctagcaccatgggctcgctccagccgctcgcgacgctgtacctcctgggcatgctcgtggcgtccgtgctggcggccgagaacctgtgggtgacggtgtactacggcgtgcccgtgtggaaggaggccaacaccacgctgttctgcgccagcgacgccaaggcctacgacaccgaggtgcacaacgtgtgggcgacccacgcctgcgtgccgacggaccccaacccccaggagatcgtgctggagaacgtgaccgagaacttcaacatgtggaagaacaacatggtggagcagatgcacgaggacatcatctcgctgtgggaccagtccctgaagccgtgcgtgaagctgacgcccctgtgcgtgaccctggactgcaccaacgtgaaggtgacgtccaccacgtccaacacggaggagaagggggagatcaagaactgctccttcaacatcaccaccgagatccgcgacaagaagcagaaggtgtacgcgctgttctaccggctggacgtggtgccgatcgacgacaacaacaacaactccagcaactaccgcctgatcaactgcaacaccagcgcctgcacccaggcctgcccgaaggtgtccttcgagcccatccccatccactactgcgcgccggccggcttcgccatcctgaagtgcaacgacaagaagttcaacggcaccggcccctgcaagaacgtgtccaccgtgcagtgcacccacgggatcaagcccgtggtgtccacgcagctgctgctgaacggctccctggccgaggaggagatcatcatccgctccgagaacatcacgaacaacgccaagaccatcatcgtgcagctgaacgagtccgtggagatcaactgcaccaggcccaacaacaacacccgcaagtccatccggatcggccctggccaggcgttctacgccaccggcgacatcatcggcgacatccgccaggcgcactgcaacatctcgggcacgaagtggaacaagaccctgcagcaggtggcgaagaagctgcgcgagcacttcaacaacaagaccatcatcttcaagcccagctccggcggcgacctggagatcacgacccactccttcaactgccgcggcgagttcttctactgcaacacctccggcctgttcaactcgacgtggatcgggaacggcacgaagaacaacaacaacaccaacgacaccatcaccctgccctgccgcatcaagcagatcatcaacatgtggcagggcgtgggccagtgcatgtacgcgccgcccatcgagggcaagatcacctgcaagtccaacatcaccggcctgctcctgacgcgcgacggcggcaacaacaacaccaacgagaccgagatcttcaggccgggcggcggcgacatgcgcgacaactggcgctcggagctgtacaagtacaaggtggtgaagatcgagcccctgggcgtggcgccgacgcgctgcaagagacgcgtggtgggccgcagacgaaggagacgggccgtgggcatcggcgcggtgttcctgggcttcctgggagcagctggttcgacgatgggcgcagcttccatgaccctgacagtgcaggcacgcaacctgctctccggcatcgtccagcagcagtcgaacctgcttcgagcccccgaggcgcagcagcacctcctcaagctgaccgtgtggggcatcaagcagctgcaggcacgcgtgctagccgtggagcgctacctccgcgaccagcagctgctcggaatctggggctgctcgggcaagctgatctgctgcaccaacgtgccgtggaacagctcctggtccaaccgcaacctctcggagatctgggacaacatgacctggctccagtgggacaaggagatctcgaactacacccagatcatctacggcctgctggaggagtcccagaaccagcaggagaagaacgagcaggacctgctggccctggactgataatctagaggatcc

SEQ ID NO: 12 - DNA, 1986 bp, Artificial Sequence
  Description of Artificial Sequence Synthetic polynucleotide
gtcgacgctagcaccatgggctcgctccagccgctcgcgacgctgtacctcctgggcatgctcgtggcgtccgtgctggcggccgagaacctgtgggtgacggtgtactacggcgtgcccgtgtggaaggaggccaacaccacgctgttctgcgccagcgacgccaaggcctacgacaccgaggtgcacaacgtgtgggcgacccacgcctgcgtgccgacggaccccaacccccaggagatcgtgctggagaacgtgaccgagaacttcaacatgtggaagaacaacatggtggagcagatgcacgaggacatcatctcgctgtgggaccagtccctgaagccgtgcgtgaagctgacgcccctgtgcgtgaccctgaactgcaccaacgtgaacgtgacgtccaccacgtccaacacggaggagaagggggagatcaagaactgctccttcaacatcaccaccgagatccgcgacaagaagcagaaggtgtacgcgctgttctaccggctggacgtggtgccgatcgacgacaacaacaacaactccagcaactaccgcctgatcaactgcaacaccagcgcctgcacccaggcctgcccgaaggtgtccttcgagcccatccccatccactactgcgcgccggccggcttcgccatcctgaagtgcaacgacaagaagttcaacggcaccggcccctgcaagaacgtgtccaccgtgcagtgcacccacgggatcaagcccgtggtgtccacgcagctgctgctgaacggctccctggccgaggaggagatcatcatccgctccgagaacatcacgaacaacgccaagaccatcatcgtgcagctgaacgagtccgtggagatcaactgcaccaggcccaacaacaacacccgcaagtccatccggatcggccctggccaggcgttctacgccaccggcgacatcatcggcgacatccgccaggcgcactgcaacatctcgggcacgaagtggaacaagaccctgcagcaggtggcgaagaagctgcgcgagcacttcaacaacaagaccatcatcttcaagcccagctccggcggcgacctggagatcacgacccactccttcaactgccgcggcgagttcttctactgcaacacctccggcctgttcaactcgacgtggatcgggaacggcacgaagaacaacaacaacaccaacgacaccatcaccctgccctgccgcatcaagcagatcatcaacatgtggcagggcgtgggccagtgcatgtacgcgccgcccatcgagggcaagatcacctgcaagtccaacatcaccggcctgctcctgacgcgcgacggcggcaacaacaacaccaacgagaccgagatcttcaggccgggcggcggcgacatgcgcgacaactggcgctcggagctgtacaagtacaaggtggtgaagatcgagcccctgggcgtggcgccgacgcgctgcaagagacgcgtggtgggccgcagacgaaggagacgggccgtgggcatcggcgcggtgttcctgggcttcctgggagcagctggttcgacgatgggcgcagcttccatgaccctgacagtgcaggcacgcaacctgctctccggcatcgtccagcagcagtcgaacctgcttcgagcccccgaggcgcagcagcacctcctcaagctgaccgtgtggggcatcaagcagctgcaggcacgcgtgctagccgtggagcgctacctccgcgaccagcagctgctcggaatctggggctgctcgggcaagctgatctgctgcaccaacgtgccgtggaacagctcctggtccaaccgcaacctctcggagatctgggacaacatgacctggctccagtgggacaaggagatctcgaactacacccagatcatctacggcctgctggaggagtcccagaaccagcaggagaagaacgagcaggacctgctggccctggactgataatctagaggatcc

SEQ ID NO: 13 - DNA, 1986 bp, Artificial Sequence
  Description of Artificial Sequence Synthetic polynucleotide
gtcgacgctagcaccatgggctcgctccagccgctcgcgacgctgtacctcctgggcatgctcgtggcgtccgtgctggcggccgagaacctgtgggtgacggtgtactacggcgtgcccgtgtggaaggaggccaacaccacgctgttctgcgccagcgacgccaaggcctacgacaccgaggtgcacaacgtgtgggcgacccacgcctgcgtgccgacggaccccaacccccaggagatcgtgctggagaacgtgaccgagaacttcaacatgtggaagaacaacatggtggagcagatgcacgaggacatcatctcgctgtgggaccagtccctgaagccgtgcgtgaagctgacgcccctgtgcgtgaccctgaactgcaccaacgtgaacgtgacgaacaccacgaacaacacggaggagaagggggagatcaagaactgctccttcaacatcaccaccgagatccgcgacaagaagcagaaggtgtacgcgctgttctaccggctggacgtggtgccgatcgacgacaacaacaacaactccagcaactaccgcctgatcaactgcaacaccagcgcctgcacccaggcctgcccgaaggtgtccttcgagcccatccccatccactactgcgcgccggccggcttcgccatcctgaagtgcaacgacaagaagttcaacggcaccggcccctgcaagaacgtgtccaccgtgcagtgcacccacgggatcaagcccgtggtgtccacgcagctgctgctgaacggctccctggccgaggaggagatcatcatccgctccgagaacatcacgaacaacgccaagaccatcatcgtgcagctgaacgagtccgtggagatcaactgcaccaggcccaacaacaacacccgcaagtccatccggatcggccctggccaggcgttctacgccaccggcgacatcatcggcgacatccgccaggcgcactgcaacatctcgggcacgaagtggaacaagaccctgcagcaggtggcgaagaagctgcgcgagcacttcaacaacaagaccatcatcttcaagcccagctccggcggcgacctggagatcacgacccactccttcaactgccgcggcgagttcttctactgcaacacctccggcctgttcaactcgacgtggatcgggaacggcacgaagaacaacaacaacaccaacgacaccatcaccctgccctgccgcatcaagcagatcatcaacatgtggcagggcgtgggccagtgcatgtacgcgccgcccatcgagggcaagatcacctgcaagtccaacatcaccggcctgctcctgacgcgcgacggcggcaacaacaacaccaacgagaccgagatcttcaggccgggcggcggcgacatgcgcgacaactggcgctcggagctgtacaagtacaaggtggtgaagatcgagcccctgggcgtggcgccgacgcgctgcaagagacgcgtggtgggccgcagacgaaggagacgggccgtgggcatcggcgcggtgttcctgggcttcctgggagcagctggttcgacgatgggcgcagcttccatgaccctgacagtgcaggcacgcaacctgctctccggcatcgtccagcagcagtcgaacctgcttcgagcccccgaggcgcagcagcacctcctcaagctgaccgtgtggggcatcaagcagctgcaggcacgcgtgctagccgtggagcgctacctccgcgaccagcagctgctcggaatctggggctgctcgggcaagctgatctgctgcaccaacgtgccgtggaacagctcctggtccaaccgcaacctctcggagatctgggacaacatgacctggctccagtgggacaaggagatctcgaactacacccagatcatctacggcctgctggaggagtcccagaaccagcaggagaagaacgagcaggacctgctggccctggactgataatctagaggatcc

SEQ ID NO: 14 - DNA, 2025 bp, Artificial Sequence
  Description of Artificial Sequence Synthetic polynucleotide
gtcgacgccaccatgggctccctgcagcccctggccaccctgtacctgctgggcatgctggtggcctccgtgctggccgccgagaacctgtgggtgaccgtgtactacggcgtgcccgtgtggaaggaggccaacaccaccctgttctgcgcctccgacgccaaggcctacgacaccgaggtgcacaacgtgtgggccacccacgcctgcgtgcccaccgaccccaacccccaggagatcgtgctggagaacgtgaccgagaacttcaacatgtggaagaacaacatggtggagcagatgcacgaggacatcatctccctgtgggaccagtccctgaagccctgcgtgaagctgacccccctgtgcgtgaccctgaactgcaccaacgtgaacgtgaccaacaccaccaacaacaccgaggagaagggcgagatcaagaactgctccttcaacatcaccaccgagatccgcgacaagaagcagaaggtgtacgccctgttctaccgcctggacgtggtgcccatcgacgacaacaacaacaactcctccaactaccgcctgatcaactgcaacacctccgcctgcacccaggcctgccccaaggtgtccttcgagcccatccccatccactactgcgcccccgccggcttcgccatcctgaagtgcaacgacaagaagttcaacggcaccggcccctgcaagaacgtgtccaccgtgcagtgcacccacggcatcaagcccgtggtgtccacccagctgctgctgaacggctccctggccgaggaggagatcatcatccgctccgagaacatcaccaacaacgccaagaccatcatcgtgcagctgaacgagtccgtggagatcaactgcacccgccccaacgccaacacccgcaagtccatccgcatcggccccggccaggccttctacgccaccggcgacatcatcggcgacatccgccaggcccactgcaacatctccggcaccaagtggaacaagaccctgcagcaggtggccaagaagctgcgcgagcacttcaacaacaagaccatcatcttcaagccctcctccggcggcgacctggagatcaccacccactccttcaactgccgcggcgagttcttctactgcaacacctccggcctgttcaactccacctggatcggcaacggcaccaagaacaacaacaacaccaacgacaccatcaccctgccctgccgcatcaagcagatcatcaacatgtggcagggcgtgggccagtgcatgtacgccccccccatcgagggcaagatcacctgcaagtccaacatcaccggcctgctgctgacccgcgacggcggcaacaacaacaccaacgagaccgagatcttccgccccggcggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggacggctccggcctgaacgacatcttcgaggcccagaagatcgagtggcacgagtagggatcc

SEQ ID NO: 15 - DNA, 2025 bp, Artificial Sequence
  Description of Artificial Sequence Synthetic polynucleotide
gtcgacgccaccatgggctccctgcagcccctggccaccctgtacctgctgggcatgctggtggcctccgtgctggccgccgagaacctgtgggtgaccgtgtactacggcgtgcccgtgtggaaggaggccaacaccaccctgttctgcgcctccgacgccaaggcctacgacaccgaggtgcacaacgtgtgggccacccacgcctgcgtgcccaccgaccccaacccccaggagatcgtgctggagaacgtgaccgagaacttcaacatgtggaagaacaacatggtggagcagatgcacgaggacatcatctccctgtgggaccagtccctgaagccctgcgtgaagctgacccccctgtgcgtgaccctgaactgcaccaacgtgaacgtgaccaacaccaccaacaacaccgaggagaagggcgagatcaagaactgctccttcaacatcaccaccgagatccgcgacaagaagcagaaggtgtacgccctgttctaccgcctggacgtggtgcccatcgacgacaacaacaacaactcctccaactaccgcctgatcaactgcaacacctccgcctgcacccaggcctgccccaaggtgtccttcgagcccatccccatccactactgcgcccccgccggcttcgccatcctgaagtgcaacgacaagaagttcaacggcaccggcccctgcaagaacgtgtccaccgtgcagtgcacccacggcatcaagcccgtggtgtccacccagctgctgctgaacggctccctggccgaggaggagatcatcatccgctccgagaacatcaccaacaacgccaagaccatcatcgtgcagctgaacgagtccgtggagatcaactgcacccgccccaacaacaacacccgcaagtccatccgcatcggccccggccaggccttctacgccaccggcgacatcatcggcgacatccgccaggcccactgcgccatctccggcaccaagtggaacaagaccctgcagcaggtggccaagaagctgcgcgagcacttcaacaacaagaccatcatcttcaagccctcctccggcggcgacctggagatcaccacccactccttcaactgccgcggcgagttcttctactgcaacacctccggcctgttcaactccacctggatcggcaacggcaccaagaacaacaacaacaccaacgacaccatcaccctgccctgccgcatcaagcagatcatcaacatgtggcagggcgtgggccagtgcatgtacgccccccccatcgagggcaagatcacctgcaagtccaacatcaccggcctgctgctgacccgcgacggcggcaacaacaacaccaacgagaccgagatcttccgccccggcggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggacggctccggcctgaacgacatcttcgaggcccagaagatcgagtggcacgagtagggatcc

SEQ ID NO: 16 - DNA, 2004 bp, Artificial Sequence
  Description of Artificial Sequence Synthetic polynucleotide
gtcgacgctagcaccatgggctcgctccagccgctcgcgacgctgtacctcctgggcatgctcgtggcgtccgtgctggcggccgagaacctgtgggtgacggtgtactacggcgtgcccgtgtggaaggaggccaacaccacgctgttctgcgccagcgacgccaaggcctacgacaccgaggtgcacaacgtgtgggcgacccacgcctgcgtgccgacggaccccaacccccaggagatcgtgctggagaacgtgaccgagaacttcaacatgtggaagaacaacatggtggagcagatgcacgaggacatcatctcgctgtgggaccagtccctgaagccgtgcgtgaagctgacgcccctgtgcgtgaccctggactgcaccaacgtgaaggtgacgtccaccacgtccaacacggaggagaagggggagatcaagaactgctccttcaacatcaccaccgagatccgcgacaagaagcagaaggtgtacgcgctgttctaccggctggacgtggtgccgatcgacgacaacaacaacaactccagcaactaccgcctgatcaactgcaacaccagcgcctgcacccaggcctgcccgaaggtgtccttcgagcccatccccatccactactgcgcgccggccggcttcgccatcctgaagtgcaacgacaagaagttcaacggcaccggcccctgcaagaacgtgtccaccgtgcagtgcacccacgggatcaagcccgtggtgtccacgcagctgctgctgaacggctccctggccgaggaggagatcatcatccgctccgagaacatcacgaacaacgccaagaccatcatcgtgcagctgaacgagtccgtggagatcaactgcaccaggcccaacaacaacacccgcaagtccatccggatcggccctggccaggcgttctacgccaccggcgacatcatcggcgacatccgccaggcgcactgcaacatctcgggcacgaagtggaacaagaccctgcagcaggtggcgaagaagctgcgcgagcacttcaacaacaagaccatcatcttcaagcccagctccggcggcgacctggagatcacgacccactccttcaactgccgcggcgagttcttctactgcaacacctccggcctgttcaactcgacgtggatcgggaacggcacgaagaacaacaacaacaccaacgacaccatcaccctgccctgccgcatcaagcagatcatcaacatgtggcagggcgtgggccagtgcatgtacgcgccgcccatcgagggcaagatcacctgcaagtccaacatcaccggcctgctcctgacgcgcgacggcggcaacaacaacaccaacgagaccgagatcttcaggccgggcggcggcgacatgcgcgacaactggcgctcggagctgtacaagtacaaggtggtgaagatcgagcccctgggcgtggcgccgacgcgctgcaagagacgcgtggtgggccgcagacgaaggagacgggccgtgggcatcggcgcggtgttcctgggcttcctgggagcagctggttcgacgatgggcgcagcttccatgaccctgacagtgcaggcacgcaacctgctctccggcatcgtccagcagcagtcgaacctgcttcgagcccccgaggcgcagcagcacctcctcaagctgaccgtgtggggcatcaagcagctgcaggcacgcgtgctagccgtggagcgctacctccgcgaccagcagctgctcggaatctggggctgctcgggcaagctgatctgctgcaccaacgtgccgtggaacagctcctggtccaaccgcaacctctcggagatctgggacaacatgacctggctccagtgggacaaggagatctcgaactacacccagatcatctacggcctgctggaggagtcccagaaccagcaggagaagaacgagcaggacctgctggccctggacctgcctagcaccggaggatgataatctagaggatcc

SEQ ID NO: 17 - DNA, 2013 bp, Artificial Sequence
  Description of Artificial Sequence Synthetic polynucleotide
atgggctccctgcagcccctggccaccctgtacctgctgggcatgctggtggcctccgtgctggccgccgagaacctgtgggtgaccgtgtactacggcgtgcccgtgtggaaggaggccaacaccaccctgttctgcgcctccgacgccaaggcctacgacaccgaggtgcacaacgtgtgggccacccacgcctgcgtgcccaccgaccccaacccccaggagatcgtgctggagaacgtgaccgagaacttcaacatgtggaagaacaacatggtggagcagatgcacgaggacatcatctccctgtgggaccagtccctgaagccctgcgtgaagctgacccccctgtgcgtgaccctggactgcaccaacgtgaaggtgaccaacaccaccaacaacaccgaggagaagggcgagatcaagaactgctccttcaacatcaccaccgagatccgcgacaagaagcagaaggtgtacgccctgttctaccgcctggacgtggtgcccatcgacgacaacaacaacaactcctccaactaccgcctgatcaactgcaacacctccgcctgcacccaggcctgccccaaggtgtccttcgagcccatccccatccactactgcgcccccgccggcttcgccatcctgaagtgcaacgacaagaagttcaacggcaccggcccctgcaagaacgtgtccaccgtgcagtgcacccacggcatcaagcccgtggtgtccacccagctgctgctgaacggctccctggccgaggaggagatcatcatccgctccgagaacatcaccaacaacgccaagaccatcatcgtgcagctgaacgagtccgtggagatcaactgcacccgccccaacaacaacacccgcaagtccatccgcatcggccccggccaggccttctacgccaccggcgacatcatcggcgacatccgccaggcccactgcaacatctccggcaccaagtggaacaagaccctgcagcaggtggccaagaagctgcgcgagcacttcaacaacaagaccatcatcttcaagccctcctccggcggcgacctggagatcaccacccactccttcaactgccgcggcgagttcttctactgcaacacctccggcctgttcaactccacctggatcggcaacggcaccaagaacaacaacaacaccaacgacaccatcaccctgccctgccgcatcaagcagatcatcaacatgtggcagggcgtgggccagtgcatgtacgccccccccatcgagggcaagatcacctgcaagtccaacatcaccggcctgctgctgacccgcgacggcggcaacaacaacaccaacgagaccgagatcttccgccccggcggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggacggctccggcctgaacgacatcttcgaggcccagaagatcgagtggcacgagtagggatcc

SEQ ID NO: 18 - DNA, 2013 bp, Artificial Sequence
  Description of Artificial Sequence Synthetic polynucleotide
atgggctccctgcagcccctggccaccctgtacctgctgggcatgctggtggcctccgtgctggccgccgagaacctgtgggtgaccgtgtactacggcgtgcccgtgtggaaggaggccaacaccaccctgttctgcgcctccgacgccaaggcctacgacaccgaggtgcacaacgtgtgggccacccacgcctgcgtgcccaccgaccccaacccccaggagatcgtgctggagaacgtgaccgagaacttcaacatgtggaagaacaacatggtggagcagatgcacgaggacatcatctccctgtgggaccagtccctgaagccctgcgtgaagctgacccccctgtgcgtgaccctgaactgcaccaacgtgaacgtgaccagcaccaccagcaacaccgaggagaagggcgagatcaagaactgctccttcaacatcaccaccgagatccgcgacaagaagcagaaggtgtacgccctgttctaccgcctggacgtggtgcccatcgacgacaacaacaacaactcctccaactaccgcctgatcaactgcaacacctccgcctgcacccaggcctgccccaaggtgtccttcgagcccatccccatccactactgcgcccccgccggcttcgccatcctgaagtgcaacgacaagaagttcaacggcaccggcccctgcaagaacgtgtccaccgtgcagtgcacccacggcatcaagcccgtggtgtccacccagctgctgctgaacggctccctggccgaggaggagatcatcatccgctccgagaacatcaccaacaacgccaagaccatcatcgtgcagctgaacgagtccgtggagatcaactgcacccgccccaacaacaacacccgcaagtccatccgcatcggccccggccaggccttctacgccaccggcgacatcatcggcgacatccgccaggcccactgcaacatctccggcaccaagtggaacaagaccctgcagcaggtggccaagaagctgcgcgagcacttcaacaacaagaccatcatcttcaagccctcctccggcggcgacctggagatcaccacccactccttcaactgccgcggcgagttcttctactgcaacacctccggcctgttcaactccacctggatcggcaacggcaccaagaacaacaacaacaccaacgacaccatcaccctgccctgccgcatcaagcagatcatcaacatgtggcagggcgtgggccagtgcatgtacgccccccccatcgagggcaagatcacctgcaagtccaacatcaccggcctgctgctgacccgcgacggcggcaacaacaacaccaacgagaccgagatcttccgccccggcggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggacggctccggcctgaacgacatcttcgaggcccagaagatcgagtggcacgagtagggatcc

SEQ ID NO: 19 - DNA, 2013 bp, Artificial Sequence
  Description of Artificial Sequence Synthetic polynucleotide
atgggctccctgcagcccctggccaccctgtacctgctgggcatgctggtggcctccgtgctggccgccgagaacctgtgggtgaccgtgtactacggcgtgcccgtgtggaaggaggccaacaccaccctgttctgcgcctccgacgccaaggcctacgacaccgaggtgcacaacgtgtgggccacccacgcctgcgtgcccaccgaccccaacccccaggagatcgtgctggagaacgtgaccgagaacttcaacatgtggaagaacaacatggtggagcagatgcacgaggacatcatctccctgtgggaccagtccctgaagccctgcgtgaagctgacccccctgtgcgtgaccctggactgcaccaacgtgaaggtgaccagcaccaccagcaacaccgaggagaagggcgagatcaagaactgctccttcaacatcaccaccgagatccgcgacaagaagcagaaggtgtacgccctgttctaccgcctggacgtggtgcccatcgacgacaacaacaacaactcctccaactaccgcctgatcaactgcaacacctccgcctgcacccaggcctgccccaaggtgtccttcgagcccatccccatccactactgcgcccccgccggcttcgccatcctgaagtgcaacgacaagaagttcaacggcaccggcccctgcaagaacgtgtccaccgtgcagtgcacccacggcatcaagcccgtggtgtccacccagctgctgctgaacggctccctggccgaggaggagatcatcatccgctccgagaacatcaccaacaacgccaagaccatcatcgtgcagctgaacgagtccgtggagatcaactgcacccgccccaacaacaacacccgcaagtccatccgcatcggccccggccaggccttctacgccaccggcgacatcatcggcgacatccgccaggcccactgcaacatctccggcaccaagtggaacaagaccctgcagcaggtggccaagaagctgcgcgagcacttcaacaacaagaccatcatcttcaagccctcctccggcggcgacctggagatcaccacccactccttcaactgccgcggcgagttcttctactgcaacacctccggcctgttcaactccacctggatcggcaacggcaccaagaacaacaacaacaccaacgacaccatcaccctgccctgccgcatcaagcagatcatcaacatgtggcagggcgtgggccagtgcatgtacgccccccccatcgagggcaagatcacctgcaagtccaacatcaccggcctgctgctgacccgcgacggcggcaacaacaacaccaacgagaccgagatcttccgccccggcggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggacggctccggcctgaacgacatcttcgaggcccagaagatcgagtggcacgagtagggatcc

SEQ ID NO: 20 - DNA, 1973 bp, Artificial Sequence
  Description of Artificial Sequence Synthetic polynucleotide
atgggctcgctccagccgctcgcgacgctgtacctcctgggcatgctcgtggcgtccgtgctggcggccgagaacctgtgggtgacggtgtactacggcgtgcccgtgtggaaggaggccaacaccacgctgttctgcgccagcgacgccaaggcctacgacaccaaggtgcacaacgtgtgggcgacccacgcctgcgtgccgacggaccccaacccccaggagatcgtgctggagaacgtgaccgagaacttcaacatgtggaagaacaacatggtggagcagatgcacgaggacatcatctcgctgtgggaccagtccctgaagccgtgcgtgaagctgacgcccctgtgcgtgaccctgaactgcaccaacgtgaacgtgacgaacaccacgaacaacacggaggagaagggggagatcaagaactgctccttcaacatcaccaccgagatccgcgacaagaagcagaaggtgtacgcgctgttctaccggctggacgtggtgccgatcgacgacaacaacaacaactccagcaactaccgcctgatcaactgcaacaccagcgcctgcacccaggcctgcccgaaggtgtccttcgagcccatccccatccactactgcgcgccggccggcttcgccatcctgaagtgcaacgacaagaagttcaacggcaccggcccctgcaagaacgtgtccaccgtgcagtgcacccacgggatcaagcccgtggtgtccacgcagctgctgctgaacggctccctggccgaggaggagatcatcatccgctccgagaacatcacgaacaacgccaagaccatcatcgtgcagctgaacgagtccgtggagatcaactgcaccaggcccaacaacaacacccgcaagtccatccggatcggccctggccagtggttctacgccaccggcgacatcatcggcgacatccgccaggcgcactgcaacatctcgggcacgaagtggaacaagaccctgcagcaggtggcgaagaagctgcgcgagcacttcaacaacaagaccatcatcttcaagcccagctccggcggcgacctggagatcacgacccactccttcaactgccgcggcgagttcttctactgcaacacctccggcctgttcaactcgacgtggatcgggaacggcacgaagaacaacaacaacaccaacgacaccatcaccctgccctgccgcatcaagcagatcatcaacatgtggcagggcgtgggccagtgcatgtacgcgccgcccatcgagggcaagatcacctgcaagtccaacatcaccggcctgctcctgacgcgcgacggcggcaacaacaacaccaacgagaccgagatcttcaggccgggcggcggcgacatgcgcgacaactggcgctcggagctgtacaagtacaaggtggtgaagatcgagcccctgggcgtggcgccgacgcgctgcaagagacgcgtggtgggccgcagacgaaggagacgggccgtgggcatcggcgcggtgttcctgggcttcctgggagcagctggttcgacgatgggcgcagcttccatgaccctgacagtgcaggcacgcaacctgctctccggcatcgtccagcagcagtcgaacctgcttcgagcccccgaggcgcagcagcacctcctcaagctgaccgtgtggggcatcaagcagctgcaggcacgcgtgctagccgtggagcgctacctccgcgaccagcagctgctcggaatctggggctgctcgggcaagctgatctgctgcaccaacgtgccgtggaacagctcctggtccaaccgcaacctctcggagatctgggacaacatgacctggctccagtgggacaaggagatctcgaactacacccagatcatctacggcctgctggaggagtcccagaaccagcaggagaagaacgagcaggacctgctggccctggactgataatctagaggatccag

SEQ ID NO: 21 - DNA, 1905 bp, Artificial Sequence
  Description of Artificial Sequence Synthetic polynucleotide
aagcttgtcgacaccatgcgcgtgcgcggcatccagcgcaactgccagcacctgtggcgctggggcaccctgatcctgggcatgctgatgatctgctccgccgccgagaacctgtgggtgaccgtgtactacggcgtgcccgtgtggaaggaggccaacaccaccctgttctgcgcctccgacgccaaggcctacgacaccgaggtgcacaacgtgtgggccacccacgcctgcgtgcccaccgaccccaacccccaggagatcgtgctggagaacgtgaccgagaacttcaacatgtggaagaacaacatggtggagcagatgcacgaggacatcatctccctgtgggaccagtccctgaagccctgcgtgaagctgacccccctgtgcgtgaccctgaactgcaccaacgtgaacgtgaccgccaccaccaacaacaccgaggagaagggcgagatcaagaactgctccttcaacatcaccaccgagatccgcgacaagaagcagaaggtgtacgccctgttctaccgcctggacgtggtgcccatcgacgacaacaacaacaactcctccaactaccgcctgatcaactgcaacacctccgccatcacccaggcctgccccaaggtgtccttcgagcccatccccatccactactgcgcccccgccggcttcgccatcctgaagtgcaacgacaagaagttcaacggcaccggcccctgcaagaacgtgtccaccgtgcagtgcacccacggcatcaagcccgtggtgtccacccagctgctgctgaacggctccctggccgaggaggagatcatcatccgctccgagaacatcaccaacaacgccaagaccatcatcgtgcagctgaacgagtccgtggagatcaactgcacccgccccaacaacaacacccgcaagtccatccgcatcggccccggccaggccttctacgccaccggcgacatcatcggcgacatccgccaggcccactgcaacatctccggcaccaagtggaacaagaccctgcagcaggtggccaagaagctgcgcgagcacttcaacaacaagaccatcatcttcaagccctcctccggcggcgacctggagatcaccacccactccttcaactgccgcggcgagttcttctactgcaacacctccggcctgttcaactccacctggatcggcaacggcaccaagaacaacaacaacaccaacgacaccatcaccctgccctgccgcatcaagcagatcatcaacatgtggcagggcgtgggccaggccatgtacgccccccccatcgagggcaagatcacctgcaagtccaacatcaccggcctgctgctgacccgcgacggcggcaacaacaacaccaacgagaccgagatcttccgccccggcggcggcgacatgcgcgacaactggcgctccgagctgtacaagtacaaggtggtgaagatcgagcccctgggcgtggcccccaccaaggccaagctgaccgtgcaggcccgccagctgctgtccggcatcgtgcagcagcagtccaacctgctgcgcgccatcgaggcccagcagcacctgctgcagctgaccgtgtggggcatcaagcagctgcaggcccgcgtgctggccgtggagcgctacctgaaggaccagcagctggagatctgggacaacatgacctggatggagtgggagcgcgagatcaacaactacaccgacatcatctactccctgatcgaggagtcccagaaccagcaggagaagaacgagcaggagctgctggccctggacaagtgggcctccctgtggaactggttcgacatcaccaactggctgtggggcctgaacgacatcttcgaggcccagaagatcgagtggcacgagtagggatcctctaga

SEQ ID NO: 22 - DNA, 1905 bp, Artificial Sequence
  Description of Artificial Sequence Synthetic polynucleotide
aagcttgtcgacaccatgcgcgtgcgcggcatccagcgcaactgccagcacctgtggcgctggggcaccctgatcctgggcatgctgatgatctgctccgccgccgagaacctgtgggtgaccgtgtactacggcgtgcccgtgtggaaggaggccaacaccaccctgttctgcgcctccgacgccaaggcctacgacaccgaggtgcacaacgtgtgggccacccacgcctgcgtgcccaccgaccccaacccccaggagatcgtgctggagaacgtgaccgagaacttcaacatgtggaagaacaacatggtggagcagatgcacgaggacatcatctccctgtgggaccagtccctgaagccctgcgtgaagctgacccccctgtgcgtgaccctgaactgcaccaacgtgaacgtgaccaacaccaccgccaacaccgaggagaagggcgagatcaagaactgctccttcaacatcaccaccgagatccgcgacaagaagcagaaggtgtacgccctgttctaccgcctggacgtggtgcccatcgacgacaacaacaacaactcctccaactaccgcctgatcaactgcaacacctccgccatcacccaggcctgccccaaggtgtccttcgagcccatccccatccactactgcgcccccgccggcttcgccatcctgaagtgcaacgacaagaagttcaacggcaccggcccctgcaagaacgtgtccaccgtgcagtgcacccacggcatcaagcccgtggtgtccacccagctgctgctgaacggctccctggccgaggaggagatcatcatccgctccgagaacatcaccaacaacgccaagaccatcatcgtgcagctgaacgagtccgtggagatcaactgcacccgccccaacaacaacacccgcaagtccatccgcatcggccccggccaggccttctacgccaccggcgacatcatcggcgacatccgccaggcccactgcaacatctccggcaccaagtggaacaagaccctgcagcaggtggccaagaagctgcgcgagcacttcaacaacaagaccatcatcttcaagccctcctccggcggcgacctggagatcaccacccactccttcaactgccgcggcgagttcttctactgcaacacctccggcctgttcaactccacctggatcggcaacggcaccaagaacaacaacaacaccaacgacaccatcaccctgccctgccgcatcaagcagatcatcaacatgtggcagggcgtgggccaggccatgtacgccccccccatcgagggcaagatcacctgcaagtccaacatcaccggcctgctgctgacccgcgacggcggcaacaacaacaccaacgagaccgagatcttccgccccggcggcggcgacatgcgcgacaactggcgctccgagctgtacaagtacaaggtggtgaagatcgagcccctgggcgtggcccccaccaaggccaagctgaccgtgcaggcccgccagctgctgtccggcatcgtgcagcagcagtccaacctgctgcgcgccatcgaggcccagcagcacctgctgcagctgaccgtgtggggcatcaagcagctgcaggcccgcgtgctggccgtggagcgctacctgaaggaccagcagctggagatctgggacaacatgacctggatggagtgggagcgcgagatcaacaactacaccgacatcatctactccctgatcgaggagtcccagaaccagcaggagaagaacgagcaggagctgctggccctggacaagtgggcctccctgtggaactggttcgacatcaccaactggctgtggggcctgaacgacatcttcgaggcccagaagatcgagtggcacgagtagggatcctctaga

SEQ ID NO: 23 - DNA, 1905 bp, Artificial Sequence
  Description of Artificial Sequence Synthetic polynucleotide
aagcttgtcgacaccatgcgcgtgcgcggcatccagcgcaactgccagcacctgtggcgctggggcaccctgatcctgggcatgctgatgatctgctccgccgccgagaacctgtgggtgaccgtgtactacggcgtgcccgtgtggaaggaggccaacaccaccctgttctgcgcctccgacgccaaggcctacgacaccgaggtgcacaacgtgtgggccacccacgcctgcgtgcccaccgaccccaacccccaggagatcgtgctggagaacgtgaccgagaacttcaacatgtggaagaacaacatggtggagcagatgcacgaggacatcatctccctgtgggaccagtccctgaagccctgcgtgaagctgacccccctgtgcgtgaccctgcaatgcaccaacgtgcaagtgacccaaaccacccaaaacaccgaggagaagggcgagatcaagaactgctccttcaacatcaccaccgagatccgcgacaagaagcagaaggtgtacgccctgttctaccgcctggacgtggtgcccatcgacgacaacaacaacaactcctccaactaccgcctgatcaactgcaacacctccgccatcacccaggcctgccccaaggtgtccttcgagcccatccccatccactactgcgcccccgccggcttcgccatcctgaagtgcaacgacaagaagttcaacggcaccggcccctgcaagaacgtgtccaccgtgcagtgcacccacggcatcaagcccgtggtgtccacccagctgctgctgaacggctccctggccgaggaggagatcatcatccgctccgagaacatcaccaacaacgccaagaccatcatcgtgcagctgaacgagtccgtggagatcaactgcacccgccccaacaacaacacccgcaagtccatccgcatcggccccggccaggccttctacgccaccggcgacatcatcggcgacatccgccaggcccactgcaacatctccggcaccaagtggaacaagaccctgcagcaggtggccaagaagctgcgcgagcacttcaacaacaagaccatcatcttcaagccctcctccggcggcgacctggagatcaccacccactccttcaactgccgcggcgagttcttctactgcaacacctccggcctgttcaactccacctggatcggcaacggcaccaagaacaacaacaacaccaacgacaccatcaccctgccctgccgcatcaagcagatcatcaacatgtggcagggcgtgggccaggccatgtacgccccccccatcgagggcaagatcacctgcaagtccaacatcaccggcctgctgctgacccgcgacggcggcaacaacaacaccaacgagaccgagatcttccgccccggcggcggcgacatgcgcgacaactggcgctccgagctgtacaagtacaaggtggtgaagatcgagcccctgggcgtggcccccaccaaggccaagctgaccgtgcaggcccgccagctgctgtccggcatcgtgcagcagcagtccaacctgctgcgcgccatcgaggcccagcagcacctgctgcagctgaccgtgtggggcatcaagcagctgcaggcccgcgtgctggccgtggagcgctacctgaaggaccagcagctggagatctgggacaacatgacctggatggagtgggagcgcgagatcaacaactacaccgacatcatctactccctgatcgaggagtcccagaaccagcaggagaagaacgagcaggagctgctggccctggacaagtgggcctccctgtggaactggttcgacatcaccaactggctgtggggcctgaacgacatcttcgaggcccagaagatcgagtggcacgagtagggatcctctaga

SEQ ID NO: 24 - protein, 658 aa, Artificial Sequence
  Description of Artificial Sequence Synthetic polypeptide
MRVRGIQRNCQHLWRWGTLILGMLMICSAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLNCTNVNVTNTTNNTEEKGEIKNCSFNITTEIRDKKQKVYALFYRLDVVPIDDNNNNSSNYRLINCNTSAITQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQAMYAPPIEGKITCKSNITGLLLTRDGGNNNTNETEIFRPGGGDMRDNWRSELYKYKVVKIEPLGVAPTKCKERVVGRRRRRRAVGIGAVFLGFLGAAGSTMGAASITLTVQARQLLSGIVQQQSNLLRAPEAQQHLLQLTVWGIKQLQARVLAVERYLKDQQLLGIWGCSGKLICCTTVPWNSSWSNKSQDEIWDNMTWMEWEREINNYTDIIYSLIEESQNQQEKNEQELLALD

SEQ ID NO: 25 - protein, 658 aa, Artificial Sequence
  Description of Artificial Sequence Synthetic polypeptide
MRVRGIQRNCQHLWRWGTLILGMLMICSAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLNCTNVNVTNTTNNTEEKGEIKNCSFNITTEIRDKKQKVYALFYRLDVVPIDDNNNNSSNYRLINCNTSACTQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQCMYAPPIEGKITCKSNITGLLLTRDGGNNNTNETEIFRPGGGDMRDNWRSELYKYKVVKIEPLGVAPTKCKERVVGRRRRRRAVGIGAVFLGFLGAAGSTMGAASITLTVQARQLLSGIVQQQSNLLRAPEAQQHLLQLTVWGIKQLQARVLAVERYLKDQQLLGIWGCSGKLICCTTVPWNSSWSNKSQDEIWDNMTWMEWEREINNYTDIIYSLIEESQNQQEKNEQELLALD

SEQ ID NO: 26 - protein, 658 aa, Artificial Sequence
  Description of Artificial Sequence Synthetic polypeptide
MRVRGIQRNCQHLWRWGTLILGMLMICSAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLNCTNVNVTNTTNNTEEKGEIKNCSFNITTEIRDKKQKVYALFYRLDVVPIDDNNNNSSNYRLINCNTSAITQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQAMYAPPIEGKITCKSNITGLLLTRDGGNNNTNETEIFRPGGGDMRDNWRSELYKYKVVKIEPLGVAPTKCKERVVGRRRRRRAVGIGAVFLGFLGAAGSTMGAASMTLTVQARQLLSGIVQQQSNLLRAPEAQQHLLQLTVWGIKQLQARVLAVERYLKDQQLLGIWGCSGKLICCTTVPWNSSWSNKSQDEIWDNMTWMEWEREINNYTDIIYSLIEESQNQQEKNEQELLALD

SEQ ID NO: 27 - protein, 658 aa, Artificial Sequence
  Description of Artificial Sequence Synthetic polypeptide
MRVRGIQRNCQHLWRWGTLILGMLMICSAAENLWVTVYYGVPVWKEANTTLFCASDAKAYDTKVHNVWATHACVPTDPNPQEIVLENVTENFNMWKNNMVEQMHEDIISLWDQSLKPCVKLTPLCVTLNCTNVNVTNTTNNTEEKGEIKNCSFNITTEIRDKKQKVYALFYRLDVVPIDDNNNNSSNYRLINCNTSAITQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQWFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQAMYAPPIEGKITCKSNITGLLLTRDGGNNNTNETEIFRPGGGDMRDNWRSELYKYKVVKIEPLGVAPTKCKERVVGRRRRRRAVGIGAVFLGFLGAAGSTMGAASMTLTVQARQLLSGIVQQQSNLLRAPEAQQHLLQLTVWGIKQLQARVLAVERYLKDQQLLGIWGCSGKLICCTTVPWNSSWSNKSQDEIWDNMTWMEWEREINNYTDIIYSLIEESQNQQEKNEQELLALD

SEQ ID NO: 28 - protein, 658 aa, Artificial Sequence
  Description of Artificial Sequence Synthetic polypeptide
MRVRGIQRNCQHLWRWGTLILGMLMICSAAENLWVTVYYGVPVWKEANTTLFCASDAKAYDTEVRNVWATHACVPTDPNPQEIVLENVTENFNMWKNNMVEQMHEDIISLWDQSLKPCVKLTPLCVTLNCTNVNVTNTTNNTEEKGEIKNCSFNITTEIRDKKQKVYALFYRLDVVPIDDNNNNSSNYRLINCNTSAITQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQWFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQAMYAPPIEGKITCKSNITGLLLTRDGGNNNTNETEIFRPGGGDMRDNWRSELYKYKVVKIEPLGVAPTKCKERVVGRRRRRRAVGIGAVFLGFLGAAGSTMGAASMTLTVQARQLLSGIVQQQSNLLRAPEAQQHLLQLTVWGIKQLQARVLAVERYLKDQQLLGIWGCSGKLICCTTVPWNSSWSNKSQDEIWDNMTWMEWEREINNYTDIIYSLIEESQNQQEKNEQELLALD

SEQ ID NO: 29 - protein, 672 aa, Artificial Sequence
  Description of Artificial Sequence Synthetic polypeptide
VDATMGSLQPLATLYLLGMLVASVLAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLNCTNVNVTNTTNNTEEKGEIKNCSFNITTEIRDKKQKVYALFYRLDVVPIDDNNNNSSNYRLINCNTSAITQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQAMYAPPIEGKITCKSNITGLLLTRDGGNNNTNETEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGSGLNDIFEAQKIEWHE

SEQ ID NO: 30 - protein, 672 aa, Artificial Sequence
  Description of Artificial Sequence Synthetic polypeptide
VDATMGSLQPLATLYLLGMLVASVLAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLNCTNVNVTNTTNNTEEKGEIKNCSFNITTEIRDKKQKVYALFYRLDVVPIDDNNNNSSNYRLINCNTSACTQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQCMYAPPIEGKITCKSNITGLLLTRDGGNNNTNETEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGSGLNDIFEAQKIEWHE

SEQ ID NO: 31 - protein, 672 aa, Artificial Sequence
  Description of Artificial Sequence Synthetic polypeptide
VDATMGSLQPLATLYLLGMLVASVLAAENLWVTVYYGVPVWKEANTTLFCASDAKAYDTKVHNVWATHACVPTDPNPQEIVLENVTENFNMWKNNMVEQMHEDIISLWDQSLKPCVKLTPLCVTLNCTNVNVTNTTNNTEEKGEIKNCSFNITTEIRDKKQKVYALFYRLDVVPIDDNNNNSSNYRLINCNTSAITQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQWFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQAMYAPPIEGKITCKSNITGLLLTRDGGNNNTNETEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGSGLNDIFEAQKIEWHE

SEQ ID NO: 32 - protein, 672 aa, Artificial Sequence
  Description of Artificial Sequence Synthetic polypeptide
VDATMGSLQPLATLYLLGMLVASVLAAENLWVTVYYGVPVWKEANTTLFCASDAKAYDTEVRNVWATHACVPTDPNPQEIVLENVTENFNMWKNNMVEQMHEDIISLWDQSLKPCVKLTPLCVTLNCTNVNVTNTTNNTEEKGEIKNCSFNITTEIRDKKQKVYALFYRLDVVPIDDNNNNSSNYRLINCNTSAITQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQWFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQAMYAPPIEGKITCKSNITGLLLTRDGGNNNTNETEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGSGLNDIFEAQKIEWHE

SEQ ID NO: 33 - protein, 827 aa, Artificial Sequence
  Description of Artificial Sequence Synthetic polypeptide
VTVVDASTMGSLQPLATLYLLGMLVASVLAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLDCTNVKVTSTTSNTEEKGEIKNCSFNITTEIRDKKQKVYALFYRLDVVPIDDNNNNSSNYRLINCNTSACTQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQCMYAPPIEGKITCKSNITGLLLTRDGGNNNTNETEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 34 - protein, 656 aa, Artificial Sequence
  Description of Artificial Sequence Synthetic polypeptide
VDASTMGSLQPLATLYLLGMLVASVLAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLDCTNVKVTSTTSNTEEKGEIKNCSFNITTEIRDKKQKVYALFYRLDVVPIDDNNNNSSNYRLINCNTSACTQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQCMYAPPIEGKITCKSNITGLLLTRDGGNNNTNETEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 35 - protein, 656 aa, Artificial Sequence
  Description of Artificial Sequence Synthetic polypeptide
VDASTMGSLQPLATLYLLGMLVASVLAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLNCTNVNVTSTTSNTEEKGEIKNCSFNITTEIRDKKQKVYALFYRLDVVPIDDNNNNSSNYRLINCNTSACTQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQCMYAPPIEGKITCKSNITGLLLTRDGGNNNTNETEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 36 - protein, 656 aa, Artificial Sequence
  Description of Artificial Sequence Synthetic polypeptide
VDASTMGSLQPLATLYLLGMLVASVLAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLNCTNVNVTNTTNNTEEKGEIKNCSFNITTEIRDKKQKVYALFYRLDVVPIDDNNNNSSNYRLINCNTSACTQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQCMYAPPIEGKITCKSNITGLLLTRDGGNNNTNETEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 37 - protein, 672 aa, Artificial Sequence
  Description of Artificial Sequence Synthetic polypeptide
VDATMGSLQPLATLYLLGMLVASVLAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLNCTNVNVTNTTNNTEEKGEIKNCSFNITTEIRDKKQKVYALFYRLDVVPIDDNNNNSSNYRLINCNTSACTQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNANTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQCMYAPPIEGKITCKSNITGLLLTRDGGNNNTNETEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGSGLNDIFEAQKIEWHE

SEQ ID NO: 38 - protein, 672 aa, Artificial Sequence
  Description of Artificial Sequence Synthetic polypeptide
VDATMGSLQPLATLYLLGMLVASVLAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLNCTNVNVTNTTNNTEEKGEIKNCSFNITTEIRDKKQKVYALFYRLDVVPIDDNNNNSSNYRLINCNTSACTQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCAISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQCMYAPPIEGKITCKSNITGLLLTRDGGNNNTNETEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGSGLNDIFEAQKIEWHE

SEQ ID NO: 39 - protein, 662 aa, Artificial Sequence
  Description of Artificial Sequence Synthetic polypeptide
VDASTMGSLQPLATLYLLGMLVASVLAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLDCTNVKVTSTTSNTEEKGEIKNCSFNITTEIRDKKQKVYALFYRLDVVPIDDNNNNSSNYRLINCNTSACTQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQCMYAPPIEGKITCKSNITGLLLTRDGGNNNTNETEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDLPSTGG

SEQ ID NO: 40 - protein, 668 aa, Artificial Sequence
  Description of Artificial Sequence Synthetic polypeptide
MGSLQPLATLYLLGMLVASVLAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLDCTNVKVTNTTNNTEEKGEIKNCSFNITTEIRDKKQKVYALFYRLDVVPIDDNNNNSSNYRLINCNTSACTQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQCMYAPPIEGKITCKSNITGLLLTRDGGNNNTNETEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGSGLNDIFEAQKIEWHE

SEQ ID NO: 41 - protein, 668 aa, Artificial Sequence
  Description of Artificial Sequence Synthetic polypeptide
MGSLQPLATLYLLGMLVASVLAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLNCTNVNVTSTTSNTEEKGEIKNCSFNITTEIRDKKQKVYALFYRLDVVPIDDNNNNSSNYRLINCNTSACTQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQCMYAPPIEGKITCKSNITGLLLTRDGGNNNTNETEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGSGLNDIFEAQKIEWHE

SEQ ID NO: 42 - protein, 668 aa, Artificial Sequence
  Description of Artificial Sequence Synthetic polypeptide
MGSLQPLATLYLLGMLVASVLAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLDCTNVKVTSTTSNTEEKGEIKNCSFNITTEIRDKKQKVYALFYRLDVVPIDDNNNNSSNYRLINCNTSACTQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQCMYAPPIEGKITCKSNITGLLLTRDGGNNNTNETEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGSGLNDIFEAQKIEWHE

SEQ ID NO: 43 - protein, 651 aa, Artificial Sequence
  Description of Artificial Sequence Synthetic polypeptide
MGSLQPLATLYLLGMLVASVLAAENLWVTVYYGVPVWKEANTTLFCASDAKAYDTKVHNVWATHACVPTDPNPQEIVLENVTENFNMWKNNMVEQMHEDIISLWDQSLKPCVKLTPLCVTLNCTNVNVTNTTNNTEEKGEIKNCSFNITTEIRDKKQKVYALFYRLDVVPIDDNNNNSSNYRLINCNTSACTQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQWFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQCMYAPPIEGKITCKSNITGLLLTRDGGNNNTNETEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 44 - protein, 630 aa, Artificial Sequence
  Description of Artificial Sequence Synthetic polypeptide
KLVDTMRVRGIQRNCQHLWRWGTLILGMLMICSAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLNCTNVNVTATTNNTEEKGEIKNCSFNITTEIRDKKQKVYALFYRLDVVPIDDNNNNSSNYRLINCNTSAITQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQAMYAPPIEGKITCKSNITGLLLTRDGGNNNTNETEIFRPGGGDMRDNWRSELYKYKVVKIEPLGVAPTKAKLTVQARQLLSGIVQQQSNLLRAIEAQQHLLQLTVWGIKQLQARVLAVERYLKDQQLEIWDNMTWMEWEREINNYTDIIYSLIEESQNQQEKNEQELLALDKWASLWNWFDITNWLWGLNDIFEAQKIEWHE

SEQ ID NO: 45 - protein, 630 aa, Artificial Sequence
  Description of Artificial Sequence Synthetic polypeptide
KLVDTMRVRGIQRNCQHLWRWGTLILGMLMICSAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLNCTNVNVTNTTANTEEKGEIKNCSFNITTEIRDKKQKVYALFYRLDVVPIDDNNNNSSNYRLINCNTSAITQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQAMYAPPIEGKITCKSNITGLLLTRDGGNNNTNETEIFRPGGGDMRDNWRSELYKYKVVKIEPLGVAPTKAKLTVQARQLLSGIVQQQSNLLRAIEAQQHLLQLTVWGIKQLQARVLAVERYLKDQQLEIWDNMTWMEWEREINNYTDIIYSLIEESQNQQEKNEQELLALDKWASLWNWFDITNWLWGLNDIFEAQKIEWHE

SEQ ID NO: 46 - protein, 630 aa, Artificial Sequence
  Description of Artificial Sequence Synthetic polypeptide
KLVDTMRVRGIQRNCQHLWRWGTLILGMLMICSAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLQCTNVQVTQTTQNTEEKGEIKNCSFNITTEIRDKKQKVYALFYRLDVVPIDDNNNNSSNYRLINCNTSAITQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQAMYAPPIEGKITCKSNITGLLLTRDGGNNNTNETEIFRPGGGDMRDNWRSELYKYKVVKIEPLGVAPTKAKLTVQARQLLSGIVQQQSNLLRAIEAQQHLLQLTVWGIKQLQARVLAVERYLKDQQLEIWDNMTWMEWEREINNYTDIIYSLIEESQNQQEKNEQELLALDKWASLWNWFDITNWLWGLNDIFEAQKIEWHE

SEQ ID NO: 47 - protein, 819 aa, Artificial Sequence
  Description of Artificial Sequence Synthetic polypeptide
MGSLQPLATLYLLGMLVASVLAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLDCTNVKVTSTTSNTEEKGEIKNCSFNITTEIRDKKQKVYALFYRLDVVPIDDNNNNSSNYRLINCNTSACTQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQCMYAPPIEGKITCKSNITGLLLTRDGGNNNTNETEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 48 - protein, 647 aa, Artificial Sequence
  Description of Artificial Sequence Synthetic polypeptide
MPMGSLQPLATLYLLGMLVASVLAAENLWVTVYYGVPVWKEAKTTLFCASDAKAYEKKVHNVWATHACVPTDPNPQEMVLKNVTENFNMWKNDMVDQMHEDVISLWDQSLKPCVKLTPLCVTLNCTNATASNSSIIEGMKNCSFNITTELRDKREKKNALFYKLDIVQLDGNSSQYRLINCNTSVITQACPKVSFDPIPIHYCAPAGYAILKCNNKTFTGTGPCNNVSTVQCTHGIKPVVSTQLLLNGSLAEGEIIIRSENITNNVKTIIVHLNESVKIECTRPNNKTRTSIRIGPGQWFYATGQVIGDIREAYCNINESKWNETLQRVSKKLKEYFPHKNITFQPSSGGDLEITTHSFNCGGEFFYCNTSSLFNRTYMANSTDMANSTETNSTRTITIHCRIKQIINMWQEVGRAMYAPPIAGNITCISNITGLLLTRDGGKNNTETFRPGGGNMKDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD